(19) United States
(12) Patent Application Publication
(10) Pub. No.: US 2025/0115875 A1
(43) Pub. Date: Apr. 10, 2025

(54) COMPOSITIONS AND METHODS FOR CELL ENGINEERING FOR MEAT PRODUCTION

(71) Applicant: Fork & Goode, Inc., Jersey City, NJ (US)

(72) Inventors: Bryan J. Gonzalez, New York, NY (US); Gabor Forgacs, New York, NY (US); Niyati Gupta, New York, NY (US); Ramsey A. Foty, Chesterfield, NJ (US); Amany Emam, Jersey City, NJ (US)

(21) Appl. No.: 18/882,049

(22) Filed: Sep. 11, 2024

Related U.S. Application Data
(63) Continuation of application No. PCT/US2023/064310, filed on Mar. 14, 2023.
(60) Provisional application No. 63/320,384, filed on Mar. 16, 2022.

(51) Int. Cl. C12N 5/077; C12N 9/22; C12N 15/11; C12N 15/90

(52) U.S. Cl. CPC C12N 5/0658; C12N 9/22; C12N 15/11; C12N 15/111; C12N 15/907; C12N 2310/20; C12N 2510/04; C12N 2513/00

(57) ABSTRACT

Disclosed herein are methods and compositions for immortalization of animal cells. Immortal animal cells may have genome modifications to increase proliferative capacity, 3D growth and/or suspension growth. Immortal animal cells with highly proliferative capacity may allow production of meats ex vivo. Such production of meat may lessen food chain burdens or tighten nutrition control of the meat produced.

### CROSS REFERENCE

This application is a continuation of International Application No. PCT/US2023/064310, filed Mar. 14, 2023, which claims the benefit of U.S. Patent Application No. 63/320,384, filed Mar. 16, 2022, each of which is incorporated by reference herein in its entirety.

### SEQUENCE LISTING

The instant application contains a Sequence Listing which has been submitted electronically in XML format and is hereby incorporated by reference in its entirety. Said XML copy, created on Apr. 24, 2023, is named 56395_704_301_SL.xml and is 128 KB in size.

### BACKGROUND

The present invention relates generally to the production of meat by genetically engineering animal cells. Meat production is the primary source of methane emissions and a major contributor to climate change: About 25 to 50% of all greenhouse gases produced by human, and about 43% of farmland used is contributed by meat, diary, and egg farming. Furthermore, highlighted by the COVID pandemic crisis, slaughterhouses for meat production have been hotspots for spreading the virus. An alternative route for meat production will not only lighten the burden to the environment but provide safe production of a major food product.

### SUMMARY OF THE INVENTION

Provided herein, are methods for amplifying animal primary cells. In an aspect, a method for amplifying animal primary cells comprises introducing into the genome of the animal primary cells a first genetic modification, and a second genetic modification of a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes; selecting a master cell line from the cells having the first genetic modification and the second genetic modification.

Provided herein, are methods for amplifying animal primary cells. In an aspect, a method for amplifying animal primary cells comprises introducing into the genome of the animal primary cells a first genetic modification, and a second genetic modification of a gene or homolog thereof selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A; selecting a master cell line from the cells having the first genetic modification and the second genetic modification.

In some embodiments, the method further comprises growing a subset of cells from the master cell line to a desired cell density, collecting the subset of cells after reaching the desired cell density and forming the collected cells into meat. In some embodiments, the first genetic modification increases the expression of a functional telomere reverse transcriptase (“TERT”) protein. In some embodiments, the first genetic modification comprises a gene knock-in of a genetic construct expressing a functional TERT protein. In some embodiments, the gene knock-in comprises a transfection with the genetic construct. In some embodiments, the gene knock-in is created by using a clustered regularly-interspaced short palindromic repeats-Cas9 (“CRISPR/Cas9”) and a guide RNA targeting the gene or a sleeping beauty transposon. In some embodiments, the TERT protein is constitutively overexpressed. In some embodiments, the first genetic modification is located at a genomic safe harbor site. In some embodiments, the genomic safe harbor site is selected from the group consisting of Rosa26, pH11, GAPDH, Pifs501, ACTB, AAVS1, HPRT, TBP, HMBS, and CEP112.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat, and fish.

In some embodiments, the second genetic modification comprises knocking-out a gene or homolog thereof, wherein the gene is selected from the group consisting of ATRX, PTEN, TP53, RB1, MAP2K4, CDKN1A (p21), CDKN1B (p27), and ARID1A. In some embodiments, the second genetic modification is a mutation of a gene or homolog thereof, wherein the gene is selected from the group consisting of ATRX, PTEN, TP53, RB1, MAP2K4, CDKN1A (p21), CDKN1B (p27), and ARID1A. In some embodiments, the mutation is created by using a CRISPR/Cas9 and a guide RNA targeting the gene. In some embodiments, the guide RNA comprises a single guide RNA (sgRNA) of 17-23 nucleotides. In some embodiments, the mutation is a frameshift mutation.

Provided herein, are methods for amplifying animal primary cells. In an aspect, a method for amplifying animal primary cells comprises: introducing into the genome of the animal primary cells a first genetic modification; selecting an immortal primary cell line from the animal primary cells having the first genetic modification; introducing into the genome of the immortal primary cell line a second genetic modification, wherein the second genetic modification comprises a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes; selecting a master cell line of the immortal primary cell having the first genetic modification and the second genetic modification; and growing a subset of cells from the master cell line to a desired cell density.

Provided herein, are methods for amplifying animal primary cells. In an aspect, a method for amplifying animal primary cells comprises: introducing into the genome of the animal primary cells a first genetic modification; selecting an immortal primary cell line from the animal primary cells having the first genetic modification; introducing into the genome of the immortal primary cell line a second genetic modification, wherein the second genetic modification comprises a gene or homolog thereof selected from the group consisting of FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A; selecting a master cell line of the immortal primary cell having the first genetic modification and the second genetic modification; and growing a subset of cells from the master cell line to a desired cell density.

In some embodiments, the method further comprises collecting the subset of cells after reaching the desired cell density and forming the collected cells into meat. In some embodiments, the first genetic modification increases the expression of a functional TERT protein. In some embodiments, the first genetic modification comprises a gene knock-in of a genetic construct expressing a functional TERT protein. In some embodiments, the gene knock-in comprises a transfection with the genetic construct. In some embodiments, the gene knock-in is created by using a CRISPR/Cas9 and a guide RNA targeting the gene or a sleeping beauty transposon. In some embodiments, the TERT protein is constitutively overexpressed. In some embodiments, the first genetic modification is located at a genomic safe harbor site. In some embodiments, the genomic safe harbor site is selected from the group consisting of Rosa26, pHI1, GAPDH, Pifs501, ACTB, AAVS1, HPRT, TBP, HMBS, and CEP112.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat, and fish.

In some embodiments, the second genetic modification comprises knocking-out a gene or homolog thereof, wherein the gene is selected from the group consisting of ATRX, PTEN, TP53, RB1, MAP2K4, CDKN1A (p21), CDKN1B (p27), and ARID1A. In some embodiments, the second genetic modification is a mutation of a gene or homolog thereof, wherein the gene is selected from the group consisting of ATRX, PTEN, TP53, RB1, MAP2K4, CDKN1A (p21), CDKN1B (p27), and ARID1A. In some embodiments, the mutation is created by using a CRISPR/Cas9 and a guide RNA targeting the gene. In some embodiments, the guide RNA comprises a sgRNA of 17-23 nucleotides. In some embodiments, the mutation is a frameshift mutation.

Provided herein, are master cell lines derived from animal primary cells. In some aspect, a master cell line derived from animal primary cells comprises a first genetic modification, and a second genetic modification of a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes.

Provided herein, are master cell lines derived from animal primary cells. In some aspect, a master cell line derived from animal primary cells comprises a first genetic modification, and a second genetic modification of a gene or homolog thereof selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A.

In some embodiments, the first genetic modification increases the expression of a functional TERT protein. In some embodiments, the first genetic modification comprises a gene knock-in of a genetic construct expressing a functional TERT protein. In some embodiments, the gene knock-in comprises a transfection with the genetic construct. In some embodiments, the gene knock-in is created by using a CRISPR/Cas9 and a guide RNA targeting the gene or a sleeping beauty transposon. In some embodiments, the TERT protein is constitutively overexpressed. In some embodiments, the first genetic modification is located at a genomic safe harbor site. In some embodiments, the genomic safe harbor site is selected from the group consisting of Rosa26, pHI1, GAPDH, Pifs501, ACTB, AAVS1, HPRT, TBP, HMBS, and CEP112.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat, and fish.

In some embodiments, the second genetic modification comprises knocking-out a gene or homolog thereof, wherein the gene is selected from the group consisting of ATRX, PTEN, TP53, RB1, MAP2K4, CDKN1A (p21), CDKN1B (p27), and ARID1A. In some embodiments, the second genetic modification is a mutation of a gene or homolog thereof, wherein the gene is selected from the group consisting of ATRX, PTEN, TP53, RB1, MAP2K4, CDKN1A (p21), CDKN1B (p27), and ARID1A. In some embodiments, the mutation is created by using a CRISPR/Cas9 and a guide RNA targeting the gene. In some embodiments, the guide RNA comprises a sgRNA of 17-23 nucleotides. In some embodiments, the mutation is a frameshift mutation.

Provided herein, are methods for rendering animal primary cells immortal. In an aspect, a method for rendering animal primary cells immortal comprises introducing into the animal primary cell a genetic modification rendering the animal primary cell immortal, wherein the genetic modification is located at a genomic safe harbor site.

In some embodiments, the genomic safe harbor site is selected from the group consisting of Rosa26, pHI1, GAPDH, Pifs501, ACTB, AAVS1, HPRT, TBP, HMBS, and CEP112. In some embodiments, the genetic modification increases the expression of a functional TERT protein. In some embodiments, the genetic modification comprises a gene knock-in of a genetic construct expressing a functional TERT protein. In some embodiments, the gene knock-in comprises a transfection with the genetic construct. In some embodiments, the gene knock-in is created by using a CRISPR/Cas9 and a guide RNA targeting the gene or a sleeping beauty transposon. In some embodiments, the TERT protein is constitutively overexpressed.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat and fish.

Provided herein, are immortal primary cell lines derived from animal primary cells. In some immortal primary cell line derived from animal primary cells comprises a genetic modification rendering the animal primary cells immortal; and wherein the genetic modification is located at a genomic safe harbor site.

In some embodiments, the genomic safe harbor site is selected from the group consisting of Rosa26, pHI1, GAPDH, Pifs501, ACTB, AAVS1, HPRT, TBP, HMBS, and CEP112. In some embodiments, the genetic modification increases the expression of a functional TERT protein. In some embodiments, the genetic modification comprises a gene knock-in of a genetic construct expressing a functional TERT protein. In some embodiments, the gene knock-in comprises a transfection with the genetic construct. In some embodiments, the gene knock-in is created by using a CRISPR/Cas9 and a guide RNA targeting the gene or a sleeping beauty transposon. In some embodiments, the TERT protein is constitutively overexpressed.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells and endothelial cells. In some embodiments, the animal primary cells are extracted from an animal species selected from the group consisting of pig, cow, chicken, turkey, sheep, goat and fish.

Provided herein, are methods for amplifying animal primary cells. In aspect, a method for amplifying animal primary cells comprises: introducing into the animal primary cells a genetic modification rendering the animal primary cell highly proliferative, wherein the genetic modification is located at a genomic safe harbor site; selecting a master cell line of the animal primary cell having the genetic modification.

In some embodiments, the method further comprises growing a subset of cells from the master cell line to a desired cell density, collecting the subset of cells after reaching the desired cell density and forming the collected cells into meat.

In some embodiments, the genomic safe harbor site is selected from the group consisting of Rosa26, pHI1, GAPDH, Pifs501, ACTB, AAVS1, HPRT, TBP, HMBS, and CEP112. In some embodiments, the genetic modification increases the expression of a proto-oncogene. In some embodiments, the proto-oncogene expresses a functional cMyc protein. In some embodiments, the genetic modification comprises a gene knock-in of a genetic construct expressing a functional cMyc protein. In some embodiments, the gene knock-in comprises a transfection with the genetic construct. In some embodiments, the cMyc protein is constitutively overexpressed. In some embodiments, the gene knock-in is created by using a CRISPR/Cas9 and a guide RNA targeting the gene or a sleeping beauty transposon.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat, and fish.

Provided herein, are master cell lines derived from animal primary cells. In aspect, a master cell line comprises a genetic modification rendering the master cell line highly proliferative; and wherein the genetic modification is located at a genomic safe harbor site.

In some embodiments, the genomic safe harbor site is selected from the group consisting of Rosa26, pHI1, GAPDH, Pifs501, ACTB, AAVS1, HPRT, TBP, HMBS, and CEP112. In some embodiments, the genetic modification increases the expression of a proto-oncogene. In some embodiments, the proto-oncogene expresses a functional cMyc protein. In some embodiments, the genetic modification comprises a gene knock-in of a genetic construct expressing a functional cMyc protein. In some embodiments, the gene knock-in comprises a transfection with the genetic construct. In some embodiments, the cMyc protein is constitutively overexpressed. In some embodiments, the gene knock-in is created by using a CRISPR/Cas9 and a guide RNA targeting the gene or a sleeping beauty transposon.

In some embodiments, the animal primary cells are selected from the group consisting of cartilage cells, liver cells, heart cells, kidney cells, bladder cells, and lung cells. In some embodiments, the animal primary cells are selected from the group consisting of fibroblast cells, muscle cells, fat cells, and endothelial cells. In some embodiments, the animal primary cells are extracted from an animal species selected from the group consisting of pig, cow, chicken, turkey, sheep, goat, and fish.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIGURE.” or “FIGURES.” herein), of which:

FIG. 1 depicts the experimental design to obtain a master cell line from primary animal cells by introducing gain and/or loss of function mutations using gene editing.

FIG. 2 depicts the experimental design to obtain a master cell line from primary animal cells by introducing gain of function mutation in proto-oncogene using gene editing.

FIGS. 3A-3D show the generation of a porcine cell line carrying a loss-of-function mutation in the pTEN locus. FIG. 3A depicts a cartoon schematic of the Cas9-GFP complexed with a pre-designed sgRNA to induce loss-of-function mutation at a targeted genomic region in the pTEN locus. FIG. 3B shows two representative images of porcine primary cells before transfection (control) and 24-48 hours after transfection (transfected) with the Cas9-GFP/sgRNA RNP complex targeting the pTEN locus. Selected GFP-positive cells are highlighted in circles. FIG. 3C shows two flow cytometry plots of the two populations of the cells from FIG. 3B. In the Cas9-GFP/sgRNA complex transfected cell population (transfected), about 42.8% of all cells were GFP positive. No cells were GFP positive in the control cell population (control). Gating for GFP positive cells are highlighted in red rectangles. FIG. 3D shows a chromatogram from Sanger sequencing of the clonally expanded GFP positive cells from a single cell of the transfected cell populations in FIG. 3C and identification of a pTEN WT clone and pTEN mutant clone. Arrows label the nucleotide misalignment of the Sanger sequencing read using SnapGene due to random mutations in the pTEN locus of the pTEN mutant clone.

FIGS. 4A-4E depicts a cartoon schematic of the experimental design and generation of a porcine cell line carrying a knock-in of a porcine TERT in the GAPDH locus. FIG. 4A depicts a cartoon schematic of the experimental design to generate and characterize a TERT knock-in cell line from porcine primary cells. FIG. 4B depicts a cartoon schematic of the mechanism for knocking-in the porcine TERT, carried by a donor plasmid, into the GAPDH locus using Cas9-GFP/sgRNA complex. FIG. 4C shows in silico design of two exemplary sgRNAs targeting the GAPDH gene. The nucleotide sequences for homologous recombination repair (HDR) template are shown on top of the left and right homology arm bars. The location of the PAM (protospacer adjacent motif) sequence and the two sgRNAs are labeled. Each sgRNA has 20 nucleotides. The end of the adjacent gene IFFO1 is also shown. FIG. 4D shows two representative images of porcine primary cells before transfection (control) and 24-48 hours after transfection (transfected) with the Cas9-GFP/sgRNA RNP complex targeting the GAPDH locus and the donor plasmid with the pTERT knock-in transgene construct. Selected GFP-positive transfected cells are highlighted in circles. FIG. 4E shows two flow cytometry plots of the two populations of the cells of FIG. 4D. In the Cas9-GFP/sgRNA complex transfected cell population (transfected), about 36.2% of all cells were GFP positive. No cells were GFP positive in the control cell population (control). GFP positive cells are highlighted in red rectangles.

### DETAILED DESCRIPTION OF THE INVENTION

Described herein are methods, systems, and reagents for amplifying animal primary cells or producing meats.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein and thereof, the singular forms “a,” “an,” and “the” include plural references unless the context clearly dictates otherwise.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” should be assumed to encompass the acceptable error range for the particular value. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “cell,” “cells,” and “cell population,” as used interchangeably, refer to one or more cells from an organism. The term includes progeny of a cell or cell population. Those skilled in the art will recognize that “cells” include progeny of a single cell, and there are variations between the progeny and its original parent cell due to natural, accidental, or deliberate mutation and/or change.

The term “primary cell” refers to a cell that is isolated from its parental tissue in its parental organism. A primary cell may undergo a senescence process. A primary cell may not be immortal. A primary cell may not divide or grow or proliferate indefinitely. A primary cell may have limited potential or ability to undergo self-renewal or differentiation.

The term “cell line” refers to a cell that proliferates or divides indefinitely ex vivo or outside of an organism or a host. A cell line may also refer to a cell or a group of cells that proliferates or divides, ex vivo or outside of an organism or a host, for a substantially larger extent than that of a primary cell. A cell line may be considered immortal. A cell line may not undergo cellular senescence. A cell line may not acquire its ability or potential to proliferate or divide ex vivo or outside of an organism or a host as part of the developmental program of its parental organism.

The term “primary cell line” refers to a cell line converted from a primary cell. Such a conversion may comprise any methods, systems, or reagents as described herein and thereof. In some cases, a primary cell line may also be identified as a secondary cell line as known in the art.

The term “master cell line” refers to a cell or a cell line that may give rise to a derivative or lineage cell or cell line. A derivative cell or cell line may acquire characteristics, properties, or components that are different from that of a master cell line; otherwise, the master cell line and the derivative or lineage cell or cell line are the same or substantially the same. Such acquisition of characteristics, properties, or components may comprise any methods, systems, or reagents as described herein and thereof. Such acquisition of characteristics, properties, or components may comprise genome modifications.

The term “cell growth” refers to the process in which a cell increases its mass. Such an increase may comprise an increased synthesis of cellular materials. The term “cell division” refers to the process in which an increase occurs in the number of cells. Cell division may give rise to two daughter or progeny cells. The term “cell proliferation” refers to the process of cell growth and cell division. In the normal genetic program of many cells, a cell needs to attain a critical mass through cell growth in order to undergo cell division.

The term “meat” refers to a group of cells for consumption. A meat may also comprise a group of cells or tissue isolated from an animal (also known as flesh). A meat may be edible. A meat is edible when it is consumed by a human and causes no harm. Such harm may comprise poisoning, contamination, or infection. The edibility of a meat may depend on the types or properties of the meat. For example, some meats may be cooked to be edible. Some meats, such as those from fish, may be edible without cooking. The edibility of a meat may depend on the level of toxins and contaminating organisms. In some cases, the edibility of a meat may also depend on the appetite of a person.

The term “Telomerase reverse transcriptase” or “TERT” refers to a catalytic subunit of a telomerase. Telomerase catalyzes the addition of telomeric repeats to the ends of linear chromosomes. Telomerase has at least two subunits: (1) TERT; and (2) an RNA component (TERC). Telomeric repeats may comprise TTAAGGG (SEQ ID NO. 1). TERT catalyzes the addition of the TTAAGGG sequence to the end of a chromosome by reverse transcription, using TERC as a template. The overhang of the end of the chromosome acts as a primer for the reverse transcription for TERT. Increased expression level of TERT may facilitate the growth, division, proliferation, or immortality of a cell.

The term “homolog” refers to a gene or a protein that is related to another gene or protein by a common ancestral DNA sequence. A homolog can be an ortholog or a paralog. An ortholog refers to a gene or protein that is related to another gene or protein by a speciation event. A paralog refers to a gene or protein that is related to another gene or protein by a duplication event within a genome. A paralog may be within the same species of the gene or protein it is related to. A paralog may also be in a different species of the gene or protein it is related to. In some cases, an ortholog may retain the same function. In other cases, a paralog may evolve a new function.

The term “proto-oncogene” refers to a normal or wild-type gene that has a positive impact on the proliferation of a cell. A proto-oncogene may facilitate cell division, growth, or proliferation. A proto-oncogene may also inhibit cell differentiation, quiescence, or death. A proto-oncogene may comprise any proto-oncogenes disclosed herein and hereof. The mutated form of the proto-oncogene may be an oncogene. An oncogene may cause or facilitate cancer.

The term “tumor suppressor gene” refers to a normal or wild-type gene that has a negative impact on the proliferation of a cell. A tumor suppressor gene may inhibit cell division, growth, or proliferation. A tumor suppressor gene may also facilitate cell differentiation, quiescence, or death. Mutation or loss-of-function (LOF) mutations of tumor suppressor genes may cause or facilitate cell proliferation. A tumor suppressor may comprise any tumor suppressor genes disclosed herein and hereof.

The term “phosphatase” refers to a class of hydrolases that cleave a phosphoric acid into a phosphate and an alcohol. A phosphatase may remove a phosphate group from a molecule. For example, a protein phosphatase may remove a phosphate group from a protein. A non-protein phosphatase may remove a phosphate group from a non-protein substrate.

The term “kinase” refers to a class of enzymes that catalyzes the transfer of a phosphate group from a phosphate donor to substrate. The phosphate donor may be a high energy molecule such as an ATP or GTP. A protein kinase may add or attach a phosphate group to a protein substrate. A non-protein kinase may remove a phosphate group from a non-protein substrate. In some cases, a kinase and phosphatase may act opposite to each other in the regulation of the phosphate transfer to and from the substrate.

The term “chromatin remodeler” refers to a class of proteins that modifies or rearranges the chromatin structure of a cell. A chromatin remodeler may comprise a histone remodeler that modifies histones. A chromatin remodeler may also comprise a nucleosome remodeler that modifies the nucleosome.

The term “DNA replication” refers to the process in which two identical copies of deoxyribonucleic acid (DNA) molecules are produced from a single copy of DNA molecule in a cell or an organism.

The term “cell cycle progression” refers to the process in which a cell gives rise to two daughter or progeny cells. Cell cycle progression may be used interchangeably with the term “cell proliferation” as described herein.

The terms “AKT/PKB/PI3K/mTOR pathway” refers to a highly conserved intracellular signaling pathway for cell cycle, metabolism, growth, proliferation, and survival regulation. In AKT/PKB/PI3K/mTOR pathway, activation of the receptor tyrosine kinases (e.g., insulin receptor or insulin-like growth factor receptor (IGF)) by growth factor (e.g., insulin or IGF) leads to the activation of the phosphoinositide-3-kinase (PI3K). Activated PI3K converts phosphatidylinositol (3,4,5)-trisphosphate (PIP3) from phosphatidylinositol (3,4)-bisphosphate (PIP2) in the plasma membrane. PIP3 in turns recruits Protein kinase B (PKB)/AKT to the plasma membrane, allowing 3-phosphoinositide-dependent protein kinase 1 (PDKT) to activate PKB/AKT. The activated PKB/AKT in turns activates a diverse set of substrates and effectors to directly or indirectly regulate cell cycle, metabolism, growth, proliferation, and/or survival. One such substrate is the mammalian target of rapamycin (mTOR) complex 1.

The term “cadherin family” refers to a family of transmembrane proteins that mediate cell-cell adhesion. Cadherin is a type of cell-adhesion molecules (CAMs). Cadherin may be involved in the formation of adherens junctions. Binding of calcium may regulate the function of cadherins. Cadherins may comprise intracellular signaling functions.

The term “genome modification” refers to the process of altering a genome. One type of genome modification may comprise altering the genetic sequence in the genome of a cell or organism. Altering the genetic sequence may confer a phenotypic consequence. The changes of the genome incurred by genome modification may comprise an insertion, substitution, rearrangement, or deletion of the DNA in the genome of the cell or organism. A cell or organism with genome modification may acquire characteristics, properties, or components different from those of a cell or organism lacking the genome modification. A variety of methods may be used in genome modification, comprising CRISPR/Cas or sleeping beauty transposon system.

The term “knock-in” or “knocking-in” refers to an insertion of a heterologous genetic sequence inserted into a genome. A knock-in is a type of genome modifications. A knock-in may be directed to a specific or pre-determined genetic location. A knock-in may also be directed to a random genetic location or a genetic location that is not pre-determined.

The term “knock-out” or “knocking-out” refers to a mutation or deletion of an endogenous genetic sequence in a gene of a genome that perturbs the expression of the gene affecting its function. A knock-out is a type of genome modifications.

The term “frameshift” mutation refers to any mutation that can change the reading frame of a DNA or resultant transcript RNA sequence. A frameshift mutation may be caused by an insertion or deletion of a number of nucleotides in a DNA sequence that is not divisible by three. A frameshift mutation may be caused by a substitution or rearrangement of a number of nucleotides in a DNA sequence such that the original reading frame is replaced with another reading frame.

The term “CRISPR/Cas” refers to a genome modification technique that utilizes the components of CRISPR/Cas protein bacterial antiviral defense system. CRISPR is also known as clustered regularly interspaced short palindromic repeats. CRISPR is a family of DNA sequences found in the genome of prokaryotic organism that encodes CRISPR RNAs. A Cas or (CRISPR/Cas) protein is a family of RNA-guided endonuclease. In the CRISPR/Cas protein-mediated genome modification, a guide RNA binds to an RNA-guided endonuclease and targets a specific target DNA sequence, effectively recruiting the endonuclease to the target DNA sequence. The guide RNA/endonuclease complex can cleave the target DNA sequence in the presence of a protospacer adjacent motif (PAM) sequence. A PAM sequence is a short DNA sequence downstream of the target DNA sequence. A CRISPR/Cas protein-guide RNA complex may not cleave a target DNA sequence lacking a PAM sequence. CRISPR/Cas protein-mediated genome modification may utilize guide RNAs with different secondary structures, modifications, or configuration as described herein and thereof. A Cas protein may comprise a Cas9 as an endonuclease. A Cas protein may also comprise different CRISPR-Cas classes (e.g., 1 or 2), types (e.g., I, II, III, IV, V, or VI), or subtypes.

The term “sleeping beauty transposon system” refers to an engineered transposon system (i.e., a non-viral DNA-mediated gene/genetic sequence transfer system) comprising a Sleeping Beauty transposase and a transposon/transposable element that can be inserted into a recognizable site in a genome. The transposon has a 210-250 bp inverted repeat (IRs) at each of their termini. A directly repeated DNA sequence motif (DR) is positioned at the end of each IR. A genetic sequence, such as a knock-in, may be placed between the IR/DR elements and inserted into the recognizable site in the genome by the transposase. A recognizable site may be a TA (thymine; adenine) dinucleotide.

The term “genome safe harbor” or “GSH” refers to a genomic location in a host organism that when a transgene is inserted, assures that the transgene will function predictably and/or will not directly or indirectly alter the genome of the host organism. Additionally, transgenic insertion or knock-in at GSH may not interfere with other physiological, biochemical, or genetic pathways of the host organism.

The term “animal” may comprise any organism from the kingdom Animalia. When describing a cell, the cell may be considered an animal cell if it is isolated from an animal. A cell may also be considered an animal cell if the closest counterpart of its genome is from an animal or an animal cell.

The term “fibroblast cell” or “fibroblast” refers to a cell that may produce collagens and other fibers of the extracellular matrix.

The term “muscle cell” refers to a cell that can form the muscle tissues of an animal. A muscle cell may develop sarcoplasm, sarcoplasmic reticulum, sarcosome, or sarcolemma that are specialized for muscle contraction and energy metabolism. A muscle cell may contain myofibrils and myoglobins. A muscle cell may contain a high amount of glycogen. A muscle cell may also refer to a myocyte. A muscle cell may develop from a myoblast. A muscle cell may be a cardiac muscle cell, a smooth muscle cell, or a skeletal muscle cell.

The term “fat cell” refers to a cell that can form the adipose tissues of animals. A fat cell may be an adipocyte. A fat cell may contain various sizes of fat droplets or granules. A fat cell may comprise a white adipose cell or a brown adipose cell. A white adipose cell may contain large fat droplets or granules and a small amount of cytoplasm. A brown adipose cell may contain a large amount of cytoplasm and numerous mitochondria.

The term “cartilage cell” refers to a cell that can form cartilage tissues in animals. A cartilage cell may be a chondrocyte. A cartilage cell may contain a structural component chondroitin sulfate. A cartilage cell may contain a fibrous sheath perichondrium.

The term “liver cell” refers to a cell that can form liver tissue in animals. A liver cell may be a hepatocyte. A liver cell may be a Kupfer cell. A liver cell may be a satellite cell or Ito cell. A hepatocyte may be a polygonal and contains numerous stacks of Golgi membranes. Hepatocytes may be active in synthesis of proteins and lipids. A hepatocyte may synthesize and secret very low-density lipoproteins.

The term “heart cell” refers to a cell that can form heart tissue of animals. A heart cell may be a cardiac muscle cell or a cardiomyocyte.

The term “kidney cell” refers to a cell that can form kidney tissue of animals. The kidney tissue may comprise a variety of cell types.

The term “endothelial cell” refers to a cell that can form endothelial tissue. Endothelial cells may form a single layer of cells that form a barrier between blood vessels and surrounding tissues to regulate exchanges of molecules between them.

The term “lung cell” refers to a cell that can form lung or pulmonary tissue of animals. A lung cell may be an epithelial cell. A lung cell may be a macrophage.

Cell Lines

In some instances, a method for amplifying an animal primary cell may comprise increasing the cell number and cell mass of an animal primary cell outside of a host organism. In some cases, increasing the cell number and cell mass of an animal primary cell outside of a host organism may comprise culturing the animal primary cell ex vivo. In some cases, culturing a cell ex vivo may comprise the growth or maintenance of viability of a cell, a group of cells, or a tissue outside of an organism or a host. As used herein for culturing cells, culturing a cell in vitro may also comprise culturing a cell, a group of cells, or a tissue ex vivo or outside of an organism or a host. A cell culture, in some cases, may also comprise the maintenance or induction of the differentiation (or de-differentiation) of a cell. In other cases, a cell culture may be maintained on growth media. In some cases, a cell culture may comprise solid, semi-solid, or liquid growth media. A cell culture, in some cases, may be 2-dimensional (2D) or 3-dimensional (3D). In some cases, growth media may comprise nutrients or other components required for the growth of a cell. In some cases, the types of media and the nutrients for a cell culture may depend on the cell being cultured or the application purpose of the cultured cell. In some cases, increasing the cell number and cell mass of an animal primary cell outside of a host organism may comprise culturing the animal primary cell in an artificial cell growth culture. In some cases, increasing the cell number and cell mass of an animal primary cell may comprise converting the animal primary cell into an animal primary cell line.

In some instances, an animal primary cell line may be immortal. In some cases, an animal primary cell line may divide indefinitely. In some cases, an animal primary cell line may not undergo cellular senescence. Cellular senescence, in some cases, may comprise a cessation or abortion of cell division. In some instances, an animal primary cell line may divide for a period of time before undergoing cellular senescence. In some cases, an animal primary cell line may divide for about 1 month, 3 months, 6 months, 12 months, 18 months, 24 months, 30 months, 36 months, or more months before undergoing cellular senescence. In some cases, an animal primary cell line may divide for from 1 to 3 months, from 3 to 6 months, from 6 to 12 months, from 12 to 18 months, from 18 to 24 months, from 24 to 30 months, from 30 to 36 months or more before undergoing cellular senescence. In some cases, an animal primary cell line may undergo about 15 cell divisions, 45 cell divisions, 90 cell divisions, 180 cell divisions, 270 cell divisions, 360 cell divisions, 720 or more cell divisions before undergoing cellular senescence. In some case, an animal primary cell line may undergo from 1 to 45 cell divisions, from 45 to 90 cell divisions, from 90 to 180 cell divisions, from 180 to 270 cell divisions, from 270 to 360 cell divisions, from 360 to 720 cell divisions or more before undergoing cellular senescence.

In some instances, a cell line derived from animal primary cells may undergo more cell divisions than the animal primary cell it has been derived from. In some cases, an animal primary cell line may undergo about 50%, 100%, 200%, 500%, 1000%, 2000%, 5000%, or 10000% more cell divisions than an animal primary cell does. In some cases, an animal primary cell line may undergo from 10 to 100%, from 100 to 200%, from 200 to 500%, from 500 to 1000%, from 1000 to 2000%, from 2000 to 5000%, from 5000 to 10000% more cell divisions than an animal primary cell does.

In some instances, an animal primary cell line may proliferate when it is being maintained ex vivo or in artificial growth culture. In some cases, an animal primary cell line may undergo cell growth when is being maintained ex vivo or in artificial growth culture. Cell growth, in some cases, may comprise an increase in cell mass. In some cases, an increase in cell mass may comprise an increase in cellular materials. Such cellular materials may comprise amino acids, nucleotides, carbohydrates, lipids, any derivative herein and thereof, or any combination herein and thereof. In some cases, cellular material accumulates in cell growth may also comprise organic or inorganic compounds. Non-limiting examples of nutrients may comprise Vitamin A, Vitamin C, Vitamin D, Vitamin K, α-tocopherol, thiamin, riboflavin, niacin, pantothenic acid, Vitamin B6, biotin, folate, cobalamin, choline, calcium, chloride, chromium, copper, fluoride, iodine, iron, magnesium, manganese, molybdenum, phosphorus, potassium, selenium, or sodium. In some instances, an animal primary cell line may undergo differentiation when it is being maintained ex vivo or in artificial growth culture. In some cases, such differentiation may comprise reversible or irreversible differentiation. In other cases, an animal primary cell line may undergo de-differentiation when it is being maintained ex vivo or in artificial growth culture. In some cases, an animal primary cell line may undergo cell migration when it is being maintained ex vivo or in artificial growth culture. In some cases, a cell in an animal primary cell line may be involved in cell-to-cell interaction with another cell in the primary cell line when it is being maintained ex vivo or in artificial growth culture. In some cases, a cell-cell interaction may comprise a direct or indirect cell-to-cell interaction. A direct cell-to-cell interaction may comprise a cell-to-cell adhesion between the two interacting cells. In some cases, an indirect cell-to-cell interaction may comprise intercellular signaling. Such an intercellular signaling may a signaling molecule. In some instances, an animal primary cell line may be induced to a quiescent state when it is being maintained ex vivo or in artificial growth culture. In other cases, an animal primary cell line may also be induced to secret a cellular material (e.g., growth hormones) when it is being maintained ex vivo or in artificial growth culture. The choice of what cellular process an animal primary cell line will undergo when it is being maintained ex vivo or in artificial growth culture may depend on the types, characteristics, or properties of meats the animal primary cell line may be used to produce.

### Genome Modification

Genome Modification to Convert a Cell into an Immortal Cell

In some instances, an animal primary cell may be converted to an animal primary cell line by a genome modification. In some cases, a genome modification to convert an animal primary cell to an animal primary cell line may be a first genome modification. In some cases, a genome modification may comprise altering a genome. In some cases, a genome modification may comprise altering the genetic sequence of the genetic material of a genome. In some cases, the genetic material of a genome may comprise a plurality of genes. In some cases, the genetic material of a genome may comprise nucleic acid. In some cases, nucleic acid of a genome may comprise DNA. In some instances, a genome may reside in the nucleus of a cell. In other cases, a genome may reside outside of a chromosome or nucleus. In one example, a genome may reside in the mitochondria of a cell. In some instances, the genetic sequence of an organism may comprise the nucleotide sequence of the DNA of an organism.

### Constitutive Expression and Overexpression of a Knocked-In Transgene

In some instances, a first genome modification may comprise a knock-in or insertion of DNA into a genome. In some cases, a knock-in or insertion of DNA may comprise inserting a nucleotide or sequence into a genome. The inserted nucleotide and/or sequence may create a nucleotide/sequence insertion, deletion, or substitution at the inserted gene. The inserted nucleotide and/or sequence may create a gain-of-function mutation of the inserted gene. In some cases, a knock-in or insertion of DNA may comprise inserting a heterologous sequence into a genome. In some cases, a heterologous sequence inserted into the genome may comprise a heterologous transcription unit. In some cases, a heterologous transcription unit being inserted into a genome may comprise a TERT. A heterologous transcription unit, in some cases, may comprise a coding sequence and a non-coding sequence. In some cases, a heterologous transcription unit may not comprise a non-coding sequence. A non-coding sequence, in some cases, may comprise a transcription regulatory element. In some cases, a transcription regulatory element may comprise a promoter, an enhancer, a polyadenylation sequence, an intron, an untranslated region, a micro RNA (miRNA)-regulatory region, a small hairpin RNA (shRNA)-regulatory region, a small interfering RNA (siRNA)-regulatory region, or an enhancer RNA (eRNA)-regulatory region.

In some instance, a heterologous transcription unit may not comprise a transcription regulatory element to drive the transcription of the heterologous transcription unit. In some cases, a heterologous transcription unit may use an endogenous transcription regulatory element from an endogenous gene to drive the transcription of the heterologous transcription unit. A heterologous transcription unit, in some cases, may be inserted at an endogenous genetic location. Such a genetic location may be upstream or downstream of a transcription regulatory element of an endogenous gene. When inserted at the genetic location, the transcription of a heterologous transcription may be regulated by the transcription regulatory element. In one case, the transcription of a heterologous transcription unit inserted upstream or downstream of a transcription regulatory element of an endogenous gene may be similar or the same as that of the endogenous gene.

In some instances, a heterologous transcription unit may comprise an artificial transcription regulatory element to drive the transcription of the heterologous transcription unit. Such an artificial transcription regulatory unit of a transcription unit, in some cases, may comprise the sequence of an endogenous transcriptional unit of the genome of an organism in which the heterologous transcription unit is inserted. In other cases, an artificial transcription regulatory unit of a transcription unit may comprise the sequence of an endogenous transcriptional unit other than the genome of an organism in which the heterologous transcription unit is inserted.

In some instances, a transcription regulatory sequence may comprise a promoter. A promoter, in some cases, may comprise a constitutive promoter. In some cases, a constitutive promoter may drive the expression of a gene, genetic sequence, or transcription unit at a constant level. A constant level, in some cases, may mean a level maintained within about 100%, 90%, 80%, 70%, 60%, 50%, 40%, 30%, 20%, 100%, 9%, 8%, 7%, 6%, 5%, 4%, 3%, 2%, 1%, or less. In some cases, a constant level may comprise measuring two levels, wherein the second level measured later is within about 100%, 90%, 80%, 70%, 60%, 50%, 40%, 30%, 20%, 10%, 9%, 8%, 7%, 6%, 5%, 4%, 3%, 2%, 1%, or less of the first level measured earlier. In some cases, a constitutive promoter may facilitate the expression of a gene, genetic sequence, or transcription unit independently from cellular stress, environmental fluctuation, nutrient fluctuation, cell cycle, cellular differentiation, cellular health, cellular interaction, or mortality/immortality. In some cases, a constitutive promoter may facilitate the expression of a gene, genetic sequence, or transcription unit whether a cell is cultured or maintained in vitro or ex vivo. In other cases, a constitutive promoter may facilitate the expression of a gene, genetic sequence, or transcription unit whether a cell is in isolation or in contact with another cell. In some cases, a constitutive promoter may facilitate the expression of a gene, genetic sequence, or transcription unit in an animal primary cell. In some cases, a constitutive promoter may facilitate the expression of a gene, genetic sequence, or transcription unit in a primary cell line. In other cases, a constitutive promoter may facilitate the expression of a gene, genetic sequence, or transcription unit in an immortal cell line.

In some instances, a promoter may facilitate the expression of a gene, genetic sequence, or transcription unit at a level. In some cases, a promoter may facilitate the expression of a gene, genetic sequence, or transcription unit at an overexpression level. In some cases, an overexpression level of a gene, genetic sequence, or transcription unit may be an expression level about 10%, 20%, 50%, 100%, 200-fold, 500-fold, 1000-fold, or more than that of a reference gene, genetic sequence, or transcription unit. In some cases, an overexpression level of a gene, genetic sequence, or transcription unit may be an expression level from 1 to 100%, from 100 to 200%, from 200 to 500%, from 500% to 1000% than that of a reference gene, genetic sequence, or transcription unit.

In some instances, a reference gene, genetic sequence, or transcription unit of a cell may have an expression level that is at about the mean level of all genes, genetic sequences, or transcription units of the cell. In some cases, a reference gene, genetic sequence, or transcription unit of a cell may have an expression level that is at about the median level of all genes, genetic sequences, or transcription units of the cell. In some cases, a reference gene, genetic sequence, or transcription unit of a cell may have an expression level that is at about the 50^{th} percentile of the expression level of all genes, genetic sequences, or transcription units of the cell. In some instances, the expression level of a gene, genetic sequence, or transcription unit may be measured as the level of the transcriptional product of the gene, genetic sequence, or transcription unit. In some cases, the expression level of a gene, genetic sequence, or transcription unit may be measured as the level of the translational product of the gene, genetic sequence, or transcription unit. In other cases, the expression level of a gene, genetic sequence, or transcription unit may also be measured as the rate of the transcriptional initiation, elongation, or termination of the gene, genetic sequence, or transcription unit. In some cases, the level of a gene, genetic sequence, or transcription unit may be measured at a steady level. In some cases, the level of a gene, genetic sequence, or transcription unit may be measured at a relative level. A relative level, in some case, may be normalized to a gene. In other cases, a reference gene may also be a housekeeping gene.

A housekeeping gene, in some cases, may be constitutively expressed endogenously. In some cases, a housekeeping gene may also be expressed at an overexpression level. In some cases, a housekeeping gene may comprise Rosa26, pH11, GAPDH, Pifs501, ACTB, AAVS1, HPRT, TBP, HMBS or CEP112. In some cases, a housekeeping gene may comprise Rosa26. In some cases, a housekeeping gene may comprise pH11. In some cases, a housekeeping gene may comprise GAPDH. In some cases, a housekeeping gene may comprise Pifs501. In some cases, a housekeeping gene may comprise ACTB. In some cases, a housekeeping gene may comprise AAVS1. In some cases, a housekeeping gene may comprise HPRT. In some cases, a housekeeping gene may comprise TBP. In some cases, a housekeeping gene may comprise HMBS. In some cases, a housekeeping gene may comprise CEP 112. In some instances, a constitutive promoter that facilitates the expression of a genetic sequence of a housekeeping gene may comprise the promoter sequence of a housekeeping gene. In some cases, a housekeeping gene may comprise a homolog of Rosa26, pH11, GAPDH, Pifs501, ACTB, AAVS1, HPRT, TBP, HMBS or CEP 112. In some cases, a housekeeping gene may also comprise Ccr5, EF1 alpha, or their homologs. In some cases, a constitutive promoter may also comprise Cauliflower mosaic virus (CaMV) 35S, opine promoters, plant ubiquitin (Ubi), rice actin 1 (Act-1), Human cytomegalovirus (CMV) promoter, and maize alcohol dehydrogenase 1 (Adh-1). In some cases, a housekeeping gene may comprise any housekeeping genes annotated in the Housekeeping and Reference Transcript Atlas database (http://www.housekeeping.unicamp.br/) or their homologs. In some cases, a constitutive promoter may comprise any sequences described herein and thereof, derivatives described herein and thereof, or any combinations described herein and thereof.

In some instances, a heterologous transcription unit may be inserted at a GSH site. In some cases, a GSH site may allow the constitutive expression of a gene, genetic sequence, or transcription unit inserted at the GSH site. In some cases, a GSH site may allow the expression of a gene, genetic sequence, or a transcription unit inserted at the GSH site to arrive at an overexpression level. In some cases, a GSH site may allow the constitutive expression of a gene, genetic sequence, or a transcription unit inserted at the GSH site at an overexpression level. In some cases, a GSH may be located inside of a housekeeping gene. In some cases, a knock-in or insertion of a gene, genetic sequence, or transcription unit at a GSH inside a housekeeping gene may allow the gene, genetic sequence, or transcription unit to be regulated by the promoter or regulatory sequence of the housekeeping gene. Such a housekeeping gene may be any housekeeping genes described herein and thereof. In some cases, a knock-in or insertion of a gene, genetic sequence, or transcription unit at a GSH inside a housekeeping gene may allow the gene, genetic sequence, or transcription unit to be expressed constitutively at an overexpression level by the promoter or regulatory sequence of the housekeeping gene. In some cases, a GSH may also be outside of a gene, genetic sequence, or transcription unit. In some cases, inserting a heterologous transcription unit at a GSH site may not interfere the expression of another gene. In some cases, a GSH site may be at least 50 kilobases (kb) from the 5′end of any gene, genetic sequence, or transcription unit. In some cases, a GSH site may be inside of a gene, genetic sequence, or transcription unit. In some cases, a GSH site may be inside of the genetic sequence or transcription unit of a housekeeping gene. In some cases, a GSH site may be outside of ultra-conserved regions. A GSH site, in some cases, may also be a genetic location described in Sadelain et al., “Safe harbours for the integration of new DNA in the human genome” Nature Reviews Cancer. 2011 Dec. 1; 12(1):51-8 or Pellenz et al., “New Human Chromosomal Sites with “Safe Harbor” Potential for Targeted Transgene Insertion” Human Gene Therapy. 2019 Jul. 1; 30(7): 814-828. Each of which is herein incorporated by reference in its entirety for all purposes. Genetic knock-in: TERT

In some instances, a heterologous transcription unit comprising TERT may comprise the coding sequence of a TERT. In some cases, a heterologous transcription unit comprising TERT may comprise a functional TERT. A functional TERT, in some cases, may be a subunit of a telomerase. In some cases, expression of a telomerase or a subunit of a telomerase in an animal primary cell may increase the cell proliferation of the animal primary cell. In some cases, expression of TERT or TERC in an animal primary cell may increase the cell proliferation of the animal primary cell. In some cases, expression of TERT and TERC in an animal primary cell may increase the cell proliferation of the animal primary cell. In other cases, a heterologous transcription unit comprising TERT may comprise the non-coding sequence of a TERT. In some cases, a heterologous transcription unit comprising TERT may comprise the coding sequence and non-coding sequence of a TERT. In some cases, a heterologous transcription unit comprising TERT may comprise the coding sequence of a TERT and the non-coding sequence of another protein. The inclusion of a non-coding sequence, whether it is of a TERT or not, may depend on the optimization of expression level. For example, the inclusion of a non-coding sequence in a heterologous transcription unit comprising TERT may facilitate the constitutive expression and overexpression of the TERT transcript or protein.

In some instances, a heterologous transcription unit comprising TERT may comprise the coding or non-coding sequence of a TERT. In some cases, a heterologous transcription unit comprising TERT may comprise the coding or non-coding sequence of a TERT from the kingdom Animalia. In some cases, a heterologous transcription unit comprising TERT may comprise the coding or non-coding sequence of a TERT from the phylum Chordata. In some cases, a heterologous transcription unit comprising TERT may comprise the coding or non-coding sequence of a TERT from the class Mammalia. In some cases, a heterologous transcription unit comprising TERT may comprise the coding or non-coding sequence of a TERT from the order Artiodactyla. In some cases, a heterologous transcription unit comprising TERT may comprise the coding or non-coding sequence of a TERT from the family Suidae. In some cases, a heterologous transcription unit comprising TERT may comprise the coding or non-coding sequence of a TERT from the Sub-Family Suinae. In some cases, a heterologous transcription unit comprising TERT may comprise the coding or non-coding sequence of a TERT from the Sub-Family Suinae. In some cases, a heterologous transcription unit comprising TERT may comprise the coding or non-coding sequence of a TERT from the Genus Sus. In some cases, a TERT may comprise a pig TERT. In some cases, a heterologous transcription unit comprising TERT may comprise the coding or non-coding sequence of a domestic pig's TERT. In some cases, a heterologous transcription unit comprising TERT may comprise the coding or non-coding sequence of a wild boar's TERT. In other cases, a heterologous transcription unit comprising TERT may comprise the coding or non-coding sequence of Sus scrofa's TERT.

In some cases, a knock-in or insertion of TERT at a GSH site may drive a constitutive expression of TERT. In some cases, a knock-in or insertion of TERT at a GSH site may drive an overexpression of TERT. In some cases, a knock-in or insertion of TERT at a GSH site within Rosa26, pH1l, GAPDH, Pifs501, ACTB, AAVS1, HPRT, TBP, HMBS or CEP112 may drive a constitutive expression of TERT. In some cases, a knock-in or insertion of TERT at a GSH within Rosa26, pH11, GAPDH, Pifs501, ACTB, AAVS1, HPRT, TBP, HMBS or CEP112 may drive an overexpression of TERT. In one embodiment, a knock-in or insertion of TERT at a GSH within GAPDH may allow the expression of TERT to be regulated by the promoter or regulatory sequence of GAPDH. In one embodiment, a knock-in or insertion of TERT at a GSH within GAPDH may drive a constitutive overexpression of TERT. Such constitutive overexpression of TERT, in some cases, may convert an animal primary cell into an animal primary cell line.

### CRISPR/Cas Protein-Mediated Genome Modification

In some instances, a CRISPR/Cas protein-mediated first genome modification may comprise a CRISPR/Cas protein. In some cases, a CRISPR/Cas protein nuclease may comprise a class 1 or class 2 CRISPR/Cas protein. A class 2 CRISPR/Cas protein may comprise a type II CRISPR/Cas protein or a type V CRISPR/Cas protein. In some instances, type II CRISPR/Cas protein may comprise a subtype II-A CRISPR/Cas protein, a subtype II-B CRISPR/Cas protein, or a subtype II-C CRISPR/Cas protein. A type V CRISPR/Cas protein may comprise a subtype V-A CRISPR/Cas protein, a subtype V-B CRISPR/Cas protein, a subtype V-C CRISPR/Cas protein, a subtype V-D CRISPR/Cas protein, a subtype V-E CRISPR/Cas protein, a subtype V-F CRISPR/Cas protein, a subtype V-G CRISPR/Cas protein, a subtype V-H CRISPR/Cas protein, a subtype V-I CRISPR/Cas protein, a subtype V-K CRISPR/Cas protein, or a subtype V-U CRISPR/Cas protein. In some instances, a CRISPR/Cas protein may comprise a Cas9 protein, a Cas12 protein, a Cas14 (also referred to as Casl2f or C2c10) protein, a Cas(D protein, a Cas1 protein, a Cas2 protein, a Csn2 protein, a Cas4 protein, a functional portion of any of these, a fused protein of any of these, a derivative of any of these, or any combinations herein and thereof. A CRISPR/Cas protein, in some cases, may comprise a wildtype or a variant CRISPR/Cas protein, functional portion of any of these, fusion protein of any of these, or any combinations thereof. In some cases, a Cas12 protein may be a Casl2a protein (also referred to as Cpf1), a Casl2b (also referred to as C2cl) protein, Casl2c protein (also referred to as C2c3), Cas12d (also referred to as CasY) protein, a Cas12e (also referred to as CasX) protein, a Cas12g protein, a Casl2h protein, a Casl2i protein, a Casl2k protein (also referred to as C2c5). In some cases, a Cas12c protein may be a C12c4 protein, a C12c5 protein, a C12c8 protein, a C12c9 protein, a C12cl0 protein. In some instances, a Cas(D protein may be a Cas12J protein.

In some cases, a CRISPR/Cas protein as described herein may bind a guide RNA. A complex comprising a CRIPSR/Cas protein and a guide RNA may bind to a specific target DNA sequence in a genome by the guide RNA. Such a specific target DNA may comprise a GSH site as described herein and thereof. In some cases, a GSH site targeted by CRISPR/Cas protein may comprise a sequence of Rosa26, pH11, GAPDH, Pifs501, ACTB, AAVS1, HPRT, TBP, HMBS or CEP112. In some cases, the guide RNA may comprise a sequence that is complementary to a target sequence within the target DNA, thus targeting the bound CRISPR/Cas protein to a specific location within the target DNA (the target sequence). In some cases, a CRISPR/Cas protein, when targeted to the specific target DNA sequence, may create a single-strand break, two single-strand breaks, a double-strand break, two double-strand breaks, or any combinations thereof in the genome. In some cases, a CRISPR/Cas protein-guide RNA complex may make a blunt-ended double-stranded break, a 1-base pair (bp) staggered cut, a 2-bp staggered cut, a staggered cut with more than 2 base pairs, or any combination thereof in the genome. A double-strand DNA break, in some cases, may be repaired by end-joining mechanism or homologous directed repair (HDR).

In some cases, a guide RNA may comprise a single guide RNA. In some cases, a guide RNA may comprise a crRNA and a tracrRNA. A crRNA, in some cases, may comprise a targeting sequence that hybridizes to a target sequence in the target DNA sequence. In some cases, a targeting sequence of a crRNA may comprise a sequence or completement of a GSH. In some cases, a tracrRNA may comprise a sequence that can form a stem-loop structure. Such a stem-loop structure, in some cases, may bind a CRISPR/Cas protein to activate the nuclease activity of the CRISPR/Cas protein. A single guide RNA may comprise a crRNA and a tracrRNA in one RNA molecule. A double guide RNA may comprise a crRNA and a tracrRNA in two RNA molecules. In some cases, a guide RNA may comprise a targeting sequence that hybridizes to a target sequence of a gene targeted in a first, second, or subsequent genome modifications described elsewhere in this disclosure. In one case, a guide RNA may comprise targeting sequence that hybridizes to a target sequence of a TERT transcription unit. In some instances, a guide RNA may comprise 10, 11, 12, 13, 14, 15, 16, 17, 18, 19, 20, 21, 22, 23, 24, 25, 26, 26, 27, 28, 29, 30, 31, 32, 33, 34, 35, 36, 37, 38, 39, 40, 41, 42, 43, 44, 45, 46, 47, 48, 49, 50, or more nucleotides. In some cases, a single guide RNA may comprise from 10 to 50 nucleotides.

In some instances, a knock-in or insertion of DNA sequence may comprise a DNA break created by a CRISPR/Cas protein-guide RNA complex. In some cases, CRISPR/Cas protein-guide RNA complex may create a double-strand DNA break. In some cases, a double-strand DNA break may be repaired by HDR with a double strand donor DNA or a single-stranded oligonucleotide donor DNA comprising the homologous sequence surrounding the break. Repair of such break with a donor DNA, in some cases, may result in a knock-in or insertion of the donor DNA in the genetic location of the break. In some cases, a heterologous transcription unit comprising TERT may be inserted at a GSH site by: (1), creating a double strand break at the GSH site in Rosa26, pH11, GAPDH, Pifs501, ACTB, AAVS1, HPRT, TBP, HMBS or CEP112 by a CRISPR/Cas protein targeted to the GSH site by one or more single guide RNAs (e.g., 2 single guide RNAs) comprising the sequence (or complement) of the GSH site; (2), repairing the break by HDR with a donor DNA comprising the heterologous transcription unit comprising TERT and sequence (or complement) of the GSH.

In some instances, a knock-in or insertion of a gene, genetic sequence, or heterologous transcription unit at a GSH may comprise a CRISPR/Cas9. In some instances, a CRISPR/Cas9-mediated first genome modification may comprise a CRISPR/Cas9 endonuclease. In some cases, a CRISPR/Cas9 endonuclease as described herein may bind a guide RNA. A complex comprising a CRIPSR/Cas9 and a guide RNA may bind to a specific target DNA sequence in a genome by the guide RNA. Such a specific target DNA may comprise a GSH site as described herein and thereof. In some cases, a GSH site targeted by CRISPR/Cas9 endonuclease may comprise a sequence of Rosa26, pH11, GAPDH, Pifs501, ACTB, AAVS1, HPRT, TBP, HMBS or CEP112. In some cases, the guide RNA may comprise a sequence that is complementary to a target sequence within the target DNA, thus targeting the bound CRISPR/Cas9 endonuclease to a specific location within the target DNA (the target sequence). In some cases, a CRISPR/Cas9 endonuclease, when targeted to the specific target DNA sequence, may create a single-strand break, two single-strand breaks, a double-strand break, two double-strand breaks, or any combinations thereof in the genome. In some cases, a CRISPR/Cas9 endonuclease-guide RNA complex may make a blunt-ended double-stranded break, a 1-base pair (bp) staggered cut, a 2-bp staggered cut, a staggered cut with more than 2 base pairs, or any combination thereof in the genome. A double-strand DNA break, in some cases, may be repaired by end-joining mechanism or homologous directed repair (HDR).

In some cases, a guide RNA may comprise a single guide RNA. In some cases, a guide RNA may comprise a crRNA and a tracrRNA. A crRNA, in some cases, may comprise a targeting sequence that hybridizes to a target sequence in the target DNA sequence. In some cases, a targeting sequence of a crRNA may comprise a sequence or completement of a GSH. In some cases, a tracrRNA may comprise a sequence that can form a stem-loop structure. Such a stem-loop structure, in some cases, may bind a CRISPR/Cas9 endonuclease to activate the nuclease activity of the CRISPR/Cas9 endonuclease. A single guide RNA may comprise a crRNA and a tracrRNA in one RNA molecule. A double guide RNA may comprise a crRNA and a tracrRNA in two RNA molecules. In one case, a single guide RNA may comprise a TERT transcription unit. In some instances, a single guide RNA may comprise 10, 11, 12, 13, 14, 15, 16, 17, 18, 19, 20, 21, 22, 23, 24, 25, 26, 26, 27, 28, 29, 30, 31, 32, 33, 34, 35, 36, 37, 38, 39, 40, 41, 42, 43, 44, 45, 46, 47, 48, 49, 50, or more nucleotides. In some cases, a single guide RNA may comprise from 10 to 50 nucleotides.

In some instances, a knock-in or insertion of DNA sequence may comprise a DNA break created by a CRISPR/Cas9 endonuclease-guide RNA complex. In some cases, CRISPR/Cas9 endonuclease-guide RNA complex may create a double-strand DNA break. In some cases, a double-strand DNA break may be repaired by HDR with a double strand donor DNA or a single-stranded oligonucleotide donor DNA comprising the homologous sequence surrounding the break. Repair of such break with a donor DNA, in some cases, may result in a knock-in or insertion of the donor DNA in the genetic location of the break. In some cases, a heterologous transcription unit comprising TERT may be inserted at a GSH site by: (1), creating a double strand break at the GSH site in Rosa26, pH11, GAPDH, Pifs501, ACTB, AAVS1, HPRT, TBP, HMBS or CEP 112 by a CRISPR/Cas9 endonuclease targeted to the GSH site by one or more single guide RNAs (e.g., 2 single guide RNAs) comprising the sequence (or complement) of the GSH site; (2), repairing the break by HDR with a donor DNA comprising the heterologous transcription unit comprising TERT and sequence (or complement) of the GSH.

### Sleeping Beauty Transposon-Mediated Genome Modification

In some instances, a knock-in or insertion of DNA sequence may comprise a sleeping beauty transposon. A knock-in construct or genetic sequence may be inserted into a random genetic location of a genome of an animal primary cell or an animal primary cell line. In some cases, a heterologous transcription unit may be inserted into a random genetic location of the genome. The heterologous transcription unit inserted into a random genetic location of the genome by a sleeping beauty transposon may comprise TERT or cMYC.

In some instances, inserting a knock-in construct or a genetic sequence into a random genetic location may comprise placing or cloning a knock-in construct or a genetic sequence between two sleeping beauty inverted repeats. When a sleeping beauty transposase is expressed, the transposase may bind and cleave the inverted repeats, releasing the knock-in construct or the genetic sequence. The transposase may then bind and cleave the genetic location comprising the sequence of the inverted repeat, allowing the knock-in construct or the genetic sequence to integrate or insert into the genome.

In some cases, a transposon may also comprise a Tn5 transposon, a PiggyBac transposon, a Hsmar1 transposon, a Mos1 transposon, a Frog Prince transposon, or a mariner-type transposon. In some cases, a transposon may comprise a sleeping beauty transposon. In some cases, a transposon may comprise a Tn5 transposon. In some cases, a transposon may comprise a piggyBac transposon. In some cases, a transposon may comprise a Hsmar1 transposon. In some cases, a transposon may comprise a MosT transposon. In some cases, a transposon may comprise a Frog Prince transposon. In some cases, a transposon may comprise a mariner-type transposon. In some cases, a transposon may comprise a Class I transposable element or a Class II transposable element. In some cases, a transposon may comprise any derivatives of the transposons thereof.

A knock-in of a TERT- or cMYC-comprising heterologous transcription unit at a random genetic location, in some case, may allow TERT or cMYC to be constitutively expressed. In other cases, a knock-in of a TERT-comprising heterologous transcription unit at a random genetic location may allow TERT to be overexpressed. In other cases, a knock-in of a cMYC-comprising heterologous transcription unit at a random genetic location may allow cMYC to be overexpressed.

In some instances, an animal primary cell line may be further modified to increase cell proliferation by a second, third, or more genome modifications. In some cases, an animal primary cell line may be further modified to increase cell growth or cell division by a second, third, or more genome modifications. In some cases, an animal primary cell line may grow 2-dimensionally. In other cases, an animal primary cell line may be converted to grow 3-dimensionally by a second, third, or more genome modifications. In some cases, an animal primary cell line with a knock-in or insertion of a heterologous transcription unit comprising TERT and/or cMyc at a GSH site within Rosa26, pH11, GAPDH, Pifs501, ACTB, AAVS1, HPRT, TBP, HMBS or CEP112 may be further modified to increase cell proliferation by another genetic modification in other genes.

In other cases, an animal primary cell line with a knock-in or insertion of a heterologous transcription unit comprising TERT and/or cMyc inserted at a random genetic location may be further modified to increase cell proliferation by another genetic modification in other genes. In some cases, the knock-in (e.g., TERT or cMyc) may be driven by a heterologous promoter. Such promoter may comprise Rosa26, EF1A, SV40, CMV, or a combination thereof. In some cases, the promoter may also comprise pH11, GAPDH, Pifs501, ACTB, AAVS1, HPRT, TBP, HMBS or CEP 112.

In some cases, a TERT-comprising heterologous transcription unit and a cMYC-comprising heterologous transcription unit may be driven by a different promoter. In some cases, the TERT-comprising heterologous transcription unit by a first promoter and the cMYC-comprising heterologous transcription unit may be driven by a second promoter, wherein the first promoter and the second promoter may comprise any promoters as described herein. In some cases, each of the TERT-comprising heterologous transcription unit and the cMYC-comprising heterologous transcription unit may be driven by the same promoter.

### Genome Modification to Increase Cell Proliferation

In some instances, a mutation may comprise an insertion of DNA into a gene or genome. In some cases, a mutation may comprise deletion of DNA from a gene or genome. In some cases, an indel mutation may comprise an insertion or deletion of DNA from a gene or genome. In some cases, a mutation may comprise substitution of DNA in a gene or genome. In some cases, a mutation may comprise rearrangement of DNA in a gene or genome. In some cases, a mutation may be a knock-out mutation. A knock-out mutation may comprise a LOF mutation. A LOF mutation, in some cases, may be a complete LOF mutation. In some cases, a compete LOF mutation may render the resultant mutated gene product or protein lacking its endogenous function. A complete LOF mutation may comprise a mutation that eliminates the production of a gene product or protein. In other cases, a complete LOF mutation may prevent the proper folding of a gene product or protein. In some cases, a LOF mutation may comprise a frameshift mutation.

In some instances, a second, third, or more genome modification may create a frameshift mutation in a gene. In some cases, a LOF mutation may comprise a frameshift mutation. In some cases, a frameshift mutation may comprise a mutation in the coding sequence of a gene. In other cases, a frameshift mutation may comprise a mutation in the non-coding sequence of a gene. In some cases, a frameshift mutation may comprise a mutation in the coding sequence of a gene. In some cases, a frameshift mutation, whether it is an insertion, deletion, substitution, or rearrangement of genetic sequence, may alter the endogenous codon of a gene. In other cases, a frameshift mutation may not alter the endogenous codon of a gene. In some cases, a frameshift mutation may alter the open reading frame of a gene. In some cases, a frameshift mutation may alter the translation of a coding sequence or open reading frame of a gene without altering the coding sequence or open reading frame of the gene.

In some instances, a frameshift mutation may remove a start codon of a gene. A start codon, in some cases, may comprise the nucleotide sequence ATG in a gene (or an equivalent AUG in an RNA, such as an mRNA). Removal of a start codon in a gene, in some cases, may enable the translation of the gene initiates at a region outside of the start codon of the gene. For example, removal of the first ATG in a gene may enable the translation to initiate at the second ATG of the gene. Such a usage of the second ATG as a start codon in gene may not occur in the gene without the frameshift mutation. In some cases, using the second ATG as a start codon in gene may not occur in a sufficient frequency in the gene without the frameshift mutation. A sufficient frequency may comprise more than 50%, 60%, 70%, 80%, 90%, 95%, 99%, or more in a population. In some instances, a frameshift mutation may create a termination codon in a gene. In some cases, a frameshift mutation may create a termination codon.

In some instances, a frameshift mutation may create a start codon in a gene. Such a start codon, in some cases, may replace an endogenous start codon of a gene and change the reading frame of the DNA sequence. For example, a start codon may be created by a mutation in the 5′ or upstream region of an endogenous start codon. Translation may initiate at the start codon created by the mutation and bypass the endogenous start codon. The reading frame of the translation initiated by the start codon created by the mutation may be in a different reading frame from that of the endogenous start codon.

In some instances, a knock-out mutation may be created by a CRISPR/Cas9 endonuclease-guide RNA complex. In some cases, a knock-out mutation may also be created by a CRISPR/Cas protein-guide RNA complex. In some cases, a CRISPR/Cas9 endonuclease-guide RNA complex may create a DNA break(s) at a gene for a second, third, or more genome modifications. In some cases, a CRISPR/Cas protein-guide RNA complex may also create a DNA break(s) at a gene for a second, third, or more genome modifications. In some cases, a CRISPR/Cas9 endonuclease-guide RNA complex may create a double-strand DNA break(s) for a second, third, or more genome modifications. In other cases, a CRISPR/Cas protein-guide RNA complex may create a double strand DNA break(s) for a second, third, or more genome modifications. In some cases, a double-strand DNA break at a gene for a second genome modification may be repaired by HDR with a double strand donor DNA or a single-stranded oligonucleotide donor DNA comprising the homologous sequence surrounding the break. Repair of such break with a donor DNA, in some cases, may result in a knock-out or LOF mutation at the gene(s) for the second, third, or more genome modifications.

In some cases, a knock-out or frameshift mutation at the gene for the second genome modification (or third or more genome modification) may be created by: (1), creating a double strand break at the gene for the second genome modification (or third or more genome modifications) by a CRISPR/Cas9 endonuclease-guide RNA complex targeted to the gene for the second genome modification (or third or more genome modifications); (2), repairing the break by HDR with a donor DNA comprising the insertion sequence that can cause a frameshift mutation at the gene(s) for the second, third, or more genome modifications.

In other cases, a knock-out or frameshift mutation at the gene(s) for the second genome modification (or third or more genome modifications) may also be created by: (1), creating two double strand breaks at the gene for the second genome modification (or third or more genome modifications) by a CRISPR/Cas protein-guide RNA complex targeted to the gene for the second genome modification (or third or more genome modifications), wherein the two breaks encompass a nucleotide or plurality of nucleotides that when deleted, results in a frameshift mutation; (2), repairing the break by directly ligating the breaks with endogenous repair enzyme.

In other cases, a knock-out or frameshift mutation at the gene for the second genome modification (or third or more genome modifications) may also be created by: (1), creating a double strand break or two double strand breaks at the gene for the second genome modification (or third or more genome modifications) by a CRISPR/Cas9 endonuclease-guide RNA complex targeted to the gene for the second genome modification (or third or more genome modifications); (2), repairing the break by error-prone endogenous end-joining repair mechanism.

In some cases, a knock-out or frameshift mutation at the gene for the second genome modification (or third or more genome modifications) may be created by: (1), creating a double strand break at the gene for the second genome modification (or third or more genome modifications) by a CRISPR/Cas protein-guide RNA complex targeted to the gene for the second genome modification (or third or more genome modifications); (2), repairing the break by HDR with a donor DNA comprising the insertion sequence that can cause a frameshift mutation at the gene for the second genome modification (or third or more genome modifications).

In other cases, a knock-out or frameshift mutation at the gene for the second genome modification (or third or more genome modifications) may also be created by: (1), creating two double strand breaks at the gene for the second genome modification (or third or more genome modifications) by a CRISPR/Cas protein-guide RNA complex targeted to the gene for the second genome modification (or third or more genome modifications), wherein the two breaks encompass a nucleotide or plurality of nucleotides that when deleted, results in a frameshift mutation; (2), repairing the break by directly ligating the breaks with endogenous repair enzyme.

In other cases, a knock-out or frameshift mutation at the gene for the second genome modification (or third or more genome modifications) may also be created by: (1), creating a double strand break or two double strand breaks at the gene for the second genome modification (or third or more genome modifications) by a CRISPR/Cas protein-guide RNA complex targeted to the gene for the second genome modification (or third or more genome modifications); (2), repairing the break by error-prone endogenous end-joining repair mechanism.

In some instances, a second genome modification (or third or more genome modifications) in an animal primary cell line comprising a first genome modification may increase the cell proliferation of the animal primary cell line. In some cases, a second genome modification (or third or more genome modifications) may comprise a mutation in a gene that negatively regulates or inhibits cell proliferation in an animal primary cell line. In some cases, a second genome modification (or third or more genome modifications) may comprise a mutation in a gene that negatively regulates or inhibits cell growth or cell division in an animal primary cell line.

In some instances, a second genome modification (or third or more genome modifications) may comprise a mutation in a gene selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A. In some cases, a second genome modification (or third or more genome modifications) may comprise a mutation in a homolog of a gene selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A.

In some instance a second genome modification (or third or more genome modifications) may comprise a knock-out of ATRX, PTEN, TP53, RB1, MAP2K4, CDKN1A (p21), CDKN1B (p27), ARID1A, or any homolog thereof. In some cases, a second genome modification (or third or more genome modifications) may comprise a knock-out of ATRX or its homolog. In some cases, a second genome modification (or third or more genome modifications) may comprise a knock-out of PTEN or its homolog. In some cases, a second genome modification (or third or more genome modifications) may comprise a knock-out of TP53 or its homolog. In some cases, a second genome modification (or third or more genome modifications) may comprise a knock-out of RB1 or its homolog. In some cases, a second genome modification (or third or more genome modifications) may comprise a knock-out of MAP2K4 or its homolog. In some cases, a second genome modification (or third or more genome modifications) may comprise a knock-out of CDKN1A (p21) or its homolog. In some cases, a second genome modification (or third or more genome modifications) may comprise a knock-out of CDKN1B (p27) or its homolog. In some cases, a second genome modification (or third or more genome modifications) may comprise a knock-out of ARID1A or its homolog.

In some instance a second genome modification (or third or more genome modifications) may comprise a mutation in ATRX, PTEN, TP53, RB1, MAP2K4, CDKN1A (p21), CDKN1B (p27), ARID1A, or any homolog thereof. In some cases, a second genome modification (or third or more genome modifications) may comprise a mutation in ATRX or its homolog. In some cases, a second genome modification (or third or more genome modifications) may comprise a mutation in PTEN or its homolog. In some cases, a second genome modification (or third or more genome modifications) may comprise a mutation in TP53 or its homolog. In some cases, a second genome modification (or third or more genome modifications) may comprise a mutation in RB1 or its homolog. In some cases, a second genome modification (or third or more genome modifications) may comprise a mutation in MAP2K4 or its homolog. In some cases, a second genome modification (or third or more genome modifications) may comprise a mutation in CDKN1A (p21) or its homolog. In some cases, a second genome modification (or third or more genome modifications) may comprise a mutation in CDKN1B (p27) or its homolog. In some cases, a second genome modification (or third or more genome modifications) may comprise a mutation in ARID1A or its homolog.

In some instances, a second genome modification (or third or more genome modifications) in an animal primary cell line may comprise a mutation in a tumor suppressor gene, a phosphatase gene, a chromatin remodeler gene, a gene associated with DNA replication, a gene associated with cell cycle progression, a gene involved in AKT/PKB/PI3K/mTOR pathway, a kinase family gene, or a cadherin family gene. In some cases, a mutation in a tumor suppressor gene, a phosphatase gene, a chromatin remodeler gene, a gene associated with DNA replication, a gene associated with cell cycle progression, a gene involved in AKT/PKB/PI3K/mTOR pathway, a kinase family gene, or a cadherin family gene may increase the cell proliferation of an animal primary cell line comprising a first genome modification. In some cases, a mutation in a tumor suppressor gene, a phosphatase gene, a chromatin remodeler gene, a gene associated with DNA replication, a gene associated with cell cycle progression, a gene involved in AKT/PKB/PI3K/mTOR pathway, a kinase family gene, or a cadherin family gene may increase the cell growth or cell division of an animal primary cell line. In some cases, a second genome modification (or third or more genome modifications) may also comprise a mutation in a homolog of a tumor suppressor gene, a phosphatase gene, a chromatin remodeler gene, a gene associated with DNA replication, a gene associated with cell cycle progression, a gene involved in AKT/PKB/PI3K/mTOR pathway, a kinase family gene, or a cadherin family gene. In some cases, a mutation in a tumor suppressor gene, a phosphatase gene, a chromatin remodeler gene, a gene associated with DNA replication, a gene associated with cell cycle progression, a gene involved in AKT/PKB/PI3K/mTOR pathway, a kinase family gene, or a cadherin family gene may also cause an animal primary cell to become an animal primary cell line.

### Tumor Suppressor Genes

In some instances, a tumor suppressor gene may comprise p53, RB1, WT1, NF1, NF2, APC, TSC1, TSC2, DPC4/SMAD4, DCC, BRCA1, BRCA2, PTEN, STK11, MSH2, MLH1, CDH1, VHL, CDKN2A, PTCH, MEN1. In some cases, a tumor suppressor gene may comprise a homolog of p53, RB1, WT1, NF1, NF2, APC, TSC1, TSC2, DPC4/SMAD4, DCC, BRCA1, BRCA2, PTEN, STK11, MSH2, MLH1, CDH1, VHL, CDKN2A, PTCH, MEN1. In some cases, a tumor suppressor gene may comprise a tumor suppressor gene listed in (https://bioinfo.uth.edu/TSGene/).

### Phosphatase Genes

In some instances, a phosphatase gene may comprise a gene that encodes for a protein or non-protein phosphatase. In some instances, a phosphatase gene may comprise a gene that encodes for a [Myosin-light-chain]phosphatase, [pyruvate dehydrogenase (acetyl-transferring)]-phosphatase, 3′(2′),5′-bisphosphate nucleotidase, 5′-nucleotidase, 6-phosphofructo-2-kinase, acid phosphatase, acireductone synthase, alkaline phosphatase, bisphosphoglycerate mutase, bisphosphoglycerate phosphatase, fructose-2,6-bisphosphate 2-phosphatase, fructose-bisphosphatase, glucose-6-phosphatase, inorganic diphosphatase, inositol-1,4-bisphosphate 1-phosphatase, inositol-phosphate phosphatase, inositol-polyphosphate 5-phosphatase, mRNA guanylyltransferase, multiple inositol-polyphosphate phosphatase, N-acylneuraminate-9-phosphatase, phosphatidate phosphatase, phosphatidylinositol-3,4,5-trisphosphate 3-phosphatase, phosphatidylinositol-3,4,5-trisphosphate 5-phosphatase, phosphatidylinositol-3,4-bisphosphate 4-phosphatase, phosphatidylinositol-3-phosphatase, phosphoethanolamine/phosphocholine phosphatase, phosphoglycerate mutase, phosphoglycolate phosphatase, phosphoinositide 5-phosphatase, phosphomannomutase, phosphoprotein phosphatase, phosphoric Monoester Hydrolases, phosphoserine phosphatase, polynucleotide 3′-phosphatase, polynucleotide 5′-hydroxyl-kinase, polynucleotide 5′-phosphatase, protein-tyrosine-phosphatase, pseudouridine-5′-monophosphatase, pyridoxal phosphatase, or soluble epoxide hydrolase. In some instances, a phosphatase gene may comprise a gene that encodes for protein tyrosine phosphatase, protein serine/threonine phosphatase, phosphatidic acid phosphatase (PAP), inositol-1,4,5-trisphosphate 5-phosphatase, phosphoglycerate mutase, HAD-like hydrolase, Histidine acid phosphatase, inositol monophosphatase, 5′(3′)-deoxyribonucleotidase, Alkaline phosphatase, 5′-nucleotidase family, pyrimidine 5′-nucleotidase family, glucose-6-phosphatase (Glc-6-Pase), lipin family, SAC domain family, 5′-nucleotidase type 3, FBPase class 1, inositol-3,4-bisphosphate 4-phosphatase, purple acid phosphatase, type 2 lipid phosphate phosphatase, DNA 3′ phosphatase, multiple inositol-polyphosphate phosphatase, Janus family, RNA polymerase II-associated protein 2 (RPAP2), SerB family, SSU72 phosphatase, vacuolar protein sorting 29 (VPS29) family, epoxide hydrolase family, eukaryotic PMM family, a homolog of these, or a functional domain of these.

### Chromatin Remodeler Genes

In some instances, a chromatin remodeler gene may comprise a gene that encodes for a chromatin remodeler. In some cases, a chromatin remodeler may comprise a protein that mediates the covalent modification of histone proteins of the chromosome of a cell. Such a histone modification may comprise lysine acetylation, lysine and arginine methylation, serine and threonine phosphorylation, and lysine ubiquitination and sumoylation. In some cases, a chromatin remodeler may comprise a protein that mediates the structural arrangement of the chromosome of the cell. In some cases, a chromatin remodeler may comprise a protein that mediates the nucleosome of the cell. In some cases, a chromatin remodeler may comprise an ATP-dependent chromatin remodeler. In some cases, an ATP-dependent chromatin remodeler may comprise WI/SNF (switch/sucrose-non-fermenting), ISWI (imitation switch), CHD (chromodomain-helicase-DNA binding) and IN080 (inositol requiring 80).

Genes Associated with DNA Replication

In some instances, a gene associated with DNA replication may comprise any gene or homolog encoding a gene product or protein that is involved in or regulated by DNA replication. In some cases, a gene associated with DNA replication may comprise any gene or homolog encoding a gene product or protein that is involved in the replication fork formation, primer binding, elongation or termination. In some instances, a gene associated with DNA replication may comprise any gene or homolog encoding a DNA polymerase. In some cases, a gene associated with DNA replication may comprise any gene or homolog encoding a gene product or protein that is involved in the pre-replication complex formation. Such a gene product or protein may comprise the origin recognition complex (ORC). ORC may comprise ORC1, ORC2, ORC3, ORC4, ORC5, or ORC6. Such a gene product or protein may also comprise Cdc6, Cdtl, or the Mcm complex. The Mcm complex may comprise CDC7, CDK, Mcm2, Mcm3, Mcm4, Mcm5, Mcm6, Mcm7, Mcm10, or Cdc45. In some cases, a gene associated with DNA replication may comprise any gene or homolog encoding a gene product or protein that is involved in the preinitiation complex formation (e.g., S-Cdk, Cdc7, TFIIA, TFIIB, TFIID, TFIIE, TFIIF, TFIIH, Mediator complex, or chromatin remodeling complex).

Genes Associated with Cell Cycle Progression

In some instances, a gene associated with cell cycle progression may comprise any gene or homolog encoding a gene product or protein that is involved in or regulated by cell cycle progression or cell proliferation. In some cases, a gene encoding a gene product or protein that is involved in or regulated by cell cycle progression or cell proliferation may comprise any gene or homolog listed in https://cyclebase.org/CyclebaseSearch.

In some instances, a gene associated with cell cycle progression may comprise any gene or homolog encoding a gene product or protein that is involved in or regulated by AKT/PKB/PI3K/mTOR pathway. Such a gene or homolog may comprise (IGF)) by insulin, IGF, insulin receptor, IGF receptor, IRS, PISK, PTEN, PKB/AKT, PDK1, mTOR complex 1, mTOR complex 2, TSC1, TSC2, eukaryotic translation initiation factor 4E binding protein 1 (4EBP1), ribosomal protein S6 kinase, ribosomal protein S6 (S6/RPS6), DNA-dependent protein kinase (DNA-PK), eIF4E, PRAS40, Chkl, MDM2, GSK3, PFK1, or XIAP. In some cases, a gene encoding a gene product or protein that is involved in or regulated by AKT/PKB/PI3K/mTOR pathway may comprise any gene or homolog described in Hemmings et al., “PI3K-PKB/Akt pathway” Cold Spring Harb Perspect Biol. 2012 Sep. 1; 4(9):a011189, which is herein incorporated by reference in its entirety for all purposes.

### Kinase Genes

In some instances, a kinase gene may comprise any gene encoding a kinase. In some cases, a kinase may comprise a protein or non-protein kinase. In some cases, a protein kinase may comprise a conventional or atypical protein kinase. A protein kinase, in some cases, may comprise a tyrosine, serine, or a threonine kinase. A conventional protein kinase, in some cases, may comprise an AGC kinase, CAMK kinase, CK1 kinase, CMGC kinase, RGC kinase, STE kinase, TK kinase, or TKL kinase. An atypical protein kinase may comprise alpha kinase, PIKK kinase, PDHK kinase, or RIO kinase. In some cases, a non-protein kinase may comprise a lipid kinase or carbohydrate kinase. A lipid kinase may comprise phosphatidylinositol kinase (e.g., PI3K, phosphatidylinositol-4-phosphate 3-kinase, or phosphatidylinositol-4,5-bisphosphate 3-kinaseor a sphingosine kinase) or a sphingosine kinase (e.g., SK1 or SK2). A carbohydrate kinase may comprise a hexokinase or a phosphofructokinase. A non-protein kinase, in some cases, may also comprise a nucleoside-phosphate kinase, nucleoside-diphosphate kinase, creatine kinase, phosphoglycerate kinase, riboflavin kinase, dihydroxyacetone kinase, shikimate kinase, DNA kinase, RNA kinase, or thymidine kinase. A protein kinase, in some cases, may comprise any kinase or homolog listed in http://www.kinhub.org/kinases.html #.

### Cadherin Family Genes

In some instances, a cadherin family gene may comprise a gene or homolog encoding E-cadherin, N-cadherin, N-cadherin 2, P-cadherin, desmoglein, democollin, protocadherin, R-cadherin, VE-cadherin, K-cadherin, type 2 cadherin 7, type 2 cadherin 8, type 2 cadherin 18, type 2 cadherin 19, type 2 cadherin 20, type 2 cadherin 23, T1 cadherin, T2 cadherin, OB-cadherin, T-cadherin, M-cadherin, KSP-cadherin, or LI-cadherin. A cadherin family gene, in some cases, may comprise any gene or homolog described in Yagi et al., “Cadherin superfamily genes: functions, genomic organization, and neurologic diversity” Genes & Development. 2000 May 15;14(10):1169-80, which is herein incorporated by reference in its entirety for all purposes.

### Desired Cell Density

In some cases, a second genome modification (or third or more genome modifications) in an animal primary cell line may allow the animal primary cell line to grow to a desired density. A desired density, in some cases, may comprise about 1×10\hat{ }7, 2×10\hat{ }7, 5×10\vec{ }7, 1×10\hat{ }8, 2×10\hat{ }8, 5×10\hat{ }8, 1×10\vec{ }9, 2×10\hat{ }9, 5×10\hat{ }9, 1×10\hat{ }10 cells/ml. A desired density, in some cases, may comprise from 1×10\hat{ }7 to 2×10\hat{ }7, from 2×10\hat{ }7 to 5×10\hat{ }7, from 5×10\hat{ }7 to 1×10\hat{ }8, from 1×10\hat{ }8 to 2×10\hat{ }8, from 2×10\hat{ }8 to 5×10\hat{ }8, from 5×10\hat{ }8 to 1×10\hat{ }9, from 1×10\hat{ }9 to 2×10\hat{ }9, from 2×10\hat{ }9 to 5×10\hat{ }9, from 5×10\hat{ }9 to 1×10\hat{ }10 cells/ml. A desired cell density, in some cases, may also apply to the growth of an animal primary cell.

Knock-In with Proto-Oncogenes

In some instances, a genome modification may increase cell proliferation of an animal primary cell. Such a genome modification may comprise a knock-in of proto-oncogene or cMyc.

In some instances, a genome modification may increase the cell density of the animal primary cell. Such an increase of cell density may comprise arriving to a desired cell density. In some cases, a genome modification may convert an animal primary cell into an animal primary cell line. In some case, a genome modification that can increase cell proliferation of an animal primary cell, increase cell density of an animal primary cell, or convert an animal primary cell to an animal primary cell line, may comprise modifying the genome of the animal primary cell at a GSH site. In some cases, a genome modification that can increase cell proliferation of an animal primary cell, increase cell density of an animal primary cell, or convert an animal primary cell to an animal primary cell line, may comprise a knock-in or insertion of an oncogene described herein and thereof. For example, the genome modification may comprise a knock-in or insertion of an oncogene at a GSH site within Rosa26, pH11, GAPDH, Pifs501, ACTB, AAVS1, HPRT, TBP, HMBS, or CEP112 using CRISPR/Cas9-mediated genome modification as described herein and thereof. In some case, a genome modification that can increase cell proliferation of an animal primary cell, increase cell density of an animal primary cell, or convert an animal primary cell to an animal primary cell line may comprise a knock-in or insertion of a proto-oncogene at a GSH site. In some case, a proto-oncogene that can increase cell proliferation of an animal primary cell, increase cell density of an animal primary cell, or convert an animal primary cell to an animal primary cell line may comprise cMyc. In some case, a proto-oncogene that can increase cell proliferation of an animal primary cell, increase cell density of an animal primary cell, or convert an animal primary cell to an animal primary cell line may comprise RAS, KRAS, HER2, MYC (cMyc), MYCL, MYCN, Cyclin D, Cyclin E, BRAF, BCR-ABL, SRC, FOS, JUN, ABL2, Akt1, AKT2, ATF1, BCL11A, BCL2, BCL3, BCL6, BCR<CARD11, CBLB, CCND1, CCND2, CCND3, CTNNB1, DDB2, DDX6, DEK, EGFR, ELK4, ERBB2, ETV4, ETV6, EVI1, EWSR, FGFR1, FGFR1OP, FGFR2, FUS, GOLGA5, GOPC, HMGA1, HMNGA2, HRAS, IRF4, KIT, LCK, LMO2, MAF, MAFB, MAML2, MDM2, MITF, MLL, MPL, MYB, NCOA4, NFKB2, NTRK1, NRAS, NUP214, PAX8, PDGFB, PIK3CA, PIM1, PLAG1, PPARG, PTNP11, RAF1, REL, RET, ROS1, SMO, SS18, TCL1A, TET2, TFG, TLX1, TPR, OR USP6. In some cases, a genome modification that can increase cell proliferation of an animal primary cell, increase cell density of an animal primary cell, or convert an animal primary cell to an animal primary cell line may comprise a knock-in or insertion of a proto-oncogene-comprising heterologous transcription unit described herein and thereof. Knocking-in or inserting a proto-oncogene-comprising heterologous transcription unit in the genome of an animal primary cell may comprise the methods and reagents described for the knock-in or insertion of a heterologous transcription unit comprising TERT into a genome (e.g., a GSH site within Rosa26, pH11, GAPDH, Pifs501, ACTB, AAVS1, HPRT, TBP, HMBS, or CEP112) described herein and thereof. In some cases, a genome modification that can increase cell proliferation of an animal primary cell, increase cell density of an animal primary cell, or convert an animal primary cell to an animal primary cell line may comprise a knock-in or insertion of a heterologous transcription unit comprising cMyc at a GSH site within Rosa26, pH11, GAPDH, Pifs501, ACTB, AAVS1, HPRT, TBP, HMBS, or CEP112 using CRISPR/Cas9-mediated genome modification as described herein and thereof. For example, a CRISPR/Cas9 endonuclease as described herein may be targeted to a GSH site within Rosa26, pH11, GAPDH, Pifs501, ACTB, AAVS1, HPRT, TBP, HMBS, or CEP112 by a guide RNA to which it is bound. In some cases, the CRISPR/Cas9 endonuclease, when targeted to the specific target DNA sequence, may create a double-strand DNA break. The DNA break, in some cases, may be repaired by end-joining mechanism or homologous directed repair (HDR). Repair of such break with a donor DNA, in some cases, may result in a knock-in or insertion of the donor DNA in the genetic location of the break. In some cases, a heterologous transcription unit comprising cMyc may be inserted at a GSH site by: (1), creating a double strand break at the GSH site in Rosa26, pH11, GAPDH, Pifs501, ACTB, AAVS1, HPRT, TBP, HMBS or CEP112 by a CRISPR/Cas9 endonuclease targeted to the GSH site by a single guide RNA comprising the sequence (or complement) of the GSH site; (2), repairing the break by HDR with a donor DNA comprising the heterologous transcription unit comprising cMyc and sequence (or complement) of the GSH. In some cases, a genome modification that can increase cell proliferation of an animal primary cell, increase cell density of an animal primary cell, or convert an animal primary cell to an animal primary cell line may comprise a knock-in or insertion of a heterologous transcription unit comprising cMyc at a GSH site within Rosa26, pH11, GAPDH, Pifs501, ACTB, AAVS1, HPRT, TBP, HMBS, or CEP112 using CRISPR/Cas protein-mediated genome modification as described herein and thereof. For example, a CRISPR/Cas9 endonuclease as described herein may be targeted to a GSH site within Rosa26, pH11, GAPDH, Pifs501, ACTB, AAVS1, HPRT, TBP, HMBS, or CEP112 by a guide RNA to which it is bound. The DNA break, in some cases, may be repaired by end-joining mechanism or homologous directed repair (HDR). Repair of such break with a donor DNA, in some cases, may result in a knock-in or insertion of the donor DNA in the genetic location of the break. In some cases, a heterologous transcription unit comprising cMyc may be inserted at a GSH site by: (1), creating a double strand break at the GSH site in Rosa26, pHI1, GAPDH, Pifs501, ACTB, AAVS1, HPRT, TBP, HMBS or CEP112 by a CRISPR/Cas protein targeted to the GSH site by a single guide RNA comprising the sequence (or complement) of the GSH site; (2), repairing the break by HDR with a donor DNA comprising the heterologous transcription unit comprising cMyc and sequence (or complement) of the GSH.

A knock-in of a proto-oncogene/cMyc-comprising heterologous transcription unit at a GSH, in some case, may allow the proto-oncogene or cMyc to be constitutively expressed. In other cases, a knock-in of a proto-oncogene/cMyc-comprising heterologous transcription unit at a GSH may allow the proto-oncogene or cMyc to be overexpressed.

In some instances, a gene or genetic sequence may be inserted into a random genetic location of a genome of an animal primary cell or an animal primary cell line. In some cases, a heterologous transcription unit may be inserted into a random genetic location of the genome. In some cases, a proto-oncogene may be inserted into a random genetic location of the genome. In some cases, cMyc may be inserted into a random genetic location of the genome. A knock-in of a proto-oncogene or cMyc-comprising heterologous transcription unit at a random genetic location, in some case, may allow the proto-oncogene or cMyc to be constitutively expressed. In other cases, a knock-in of a proto-oncogene/cMyc-comprising heterologous transcription unit at a random genetic location may allow the proto-oncogene or cMyc to be overexpressed. In some cases, inserting any gene or heterologous transcription unit thereof into a random genetic location of the genome of any cell thereof may increase the cell proliferation, cell growth, or cell division of the cell.

In some cases, a mutation may be introduced into a proto-oncogene to increase cell proliferation of an animal primary cell. In some cases, the mutation may comprise a gain-of-function mutation. In some cases, the mutation may facilitate a conversion of a proto-oncogene into an oncogene. In some cases, the mutation may convert a proto-oncogene into an oncogene. For example, the mutation may be introduced into the KRAS locus. The introduction of the mutation thereof may comprise the CRISPR/Cas protein or the sleeping beauty transposon system.

### Selecting a Master Cell Line

In some instances, a master cell line may comprise an animal primary cell line comprising a first genome modification and a second genome modification. In some instances, a master cell line may comprise an animal primary cell line comprising a first, second, or more genome modifications. In other cases, a master cell line may comprise an animal primary cell line comprising a first genome modification or a second genome modification. In some cases, a master cell line may be immortal or highly proliferative. In some cases, a master cell line may comprise an animal primary cell line comprising a genome modification. In some cases, a master cell line may be immortal. In other cases, a master cell line may be highly proliferative. In some cases, a master cell line may be immortal and highly proliferative. In some instances, a master cell line may comprise a third or more subsequent genome modifications. The third or more subsequent genome modification(s) may comprise any genome modification thereof. For example, the third genome modification may comprise the genome modification of a first or second genome modification. In some cases, the third genome modification may comprise an of the genetic knock-out or genetic knock-in genome modifications described elsewhere in this disclosure. In some cases, the first genome modification may occur prior to or subsequent to the second modification may occur. In some cases, the genetic knock-in may occur prior to or subsequent to the genetic knock-out may occur. In some cases, order of occurrence of the first, second, third, or subsequent genome modification(s) may comprise any order.

In some instances, a master cell line may comprise a second and third genome (or more) modifications. The genome modification in the master cell line may comprise a mutation or a knock-out of one or more of the following genes ATRX, PTEN, TP53, RB1, MAP2K4, CDKN1A (p21), CDKN1B (p27), ARID1A, FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A. In some cases, the master cell line can also comprise a knock-out of one or more of the following genes ATRX, PTEN, TP53, RB1, MAP2K4, CDKN1A (p21), CDKN1B (p27), ARID1A FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A; and a knock-in in an oncogene. The oncogene may comprise Ras/KRAS or cMYC.

In some instances, a master cell line may comprise an animal primary cell comprising a genome modification. In some cases, a master cell line may not be immortal. In other cases, a master cell line may be highly proliferative. In some cases, a master cell line may not divide indefinitely but highly proliferative.

In some instances, a master cell line may be selected. In some cases, a master cell line may be selected from a group of non-master cells. In some cases, a master cell line may be selected from a group of animal primary cells. In other cases, a master cell line may need to be purified or isolated. Selection, purification, or isolation of a master cell line, in some cases, may comprise obtaining a homogenous culture of a master cell line. In some cases, a homogenous culture may comprise at least 90%, 91%, 92%, 93%, 94%, 95%, 96%, 97%, 98%, 99%, or more cells in a cell population.

In some instances, a master cell line may be sorted. A cell sorter may be used in some cases. Cell sorting, in some cases, may comprise fluorescent activated cell sorting (FACS). In other cases, magnetic activated cell sorting (MACS) may also be used. Fluorescent marker, in some cases, may comprise a fluorescent protein as a delivery marker agent described herein and thereof. In other cases, a master cell line may be isolated by microfluidic devices. For example, individual single cells from a group of animal primary cell may be sorted and grow as individual cultures. A master cell line may then be established after the individual cells grow to a certain density. Such a certain density may depend on the applications done to the cells. In some case, a certain density may comprise a desired density described herein and thereof.

In some instances, a master cell line may undergo confirmation. A confirmation, in some cases, may comprise a PCR confirmation. A PCR confirmation may comprise designing a pair of primers that can amplify a target region that can distinguish the presence or absence of a genome modification. For example, to PCR confirm a knock-in or knock-out, a pair of primers flanking the genome region where the knock-in or knock-out is located may be used to amplify genome region by PCR. The presence of a PCR product may indicate the presence of the knock-in or knock-out. In some cases, the absence of a PCR product may indicate the absence of the knock-in or knock-out. In other cases, the PCR product with the presence of the knock-in or knock-out may have a different size from the PCR product without the presence of the knock-in or knock-out.

In some instances, a confirmation may comprise sequencing a genome modification or the genome region with a genome modification. In some cases, Sanger sequencing may be used to determine the genome sequence of genome region where a genome modification should occur.

In some instances, a confirmation may comprise detecting the protein expression of a gene where a genome modification occurs. For example, a cell with a TERT successfully knocked in may constitutively express or overexpress the TERT protein. The expression level of the TERT protein may be measured by a western blot. In other cases, the TERT protein may be measured by enzyme-linked immunosorbent assay (ELISA). In some cases, a cell with a gene successfully knocked out may not express the protein encoded by the gene. In some cases, western blot or ELISA may be used to measure the expression of the protein. In some cases, the expression of a transcript may replace the expression of a protein in a confirmation assay. For example, a reverse transcription polymerase chain reaction (RT-PCR) or Quantitative real-time PCR (QPCR) assay (or variants) may be used to measure the transcript encoded by a gene or genome region.

In some instances, a confirmation may comprise measuring the activity of a gene product. In some cases, a confirmation may comprise measuring the enzymatic activity of a gene product. For example, when confirming a TERT knock-in, the telomerase activity of the TERT may be measured. In some cases, a telomerase activity assay may comprise Telomerase Activity Quantification qPCR Assay Kit (ScienCell Research Laboratories #8928) or TRAPeze® Telomerase Detection Kit (Sigma-Aldrich #S7700). In some cases, a telomerase activity may also be measured by the assays described in Stvorstov et al., “Assays for Detection of Telomerase Activity” Acta Naturae. 2011 Jan-Mar; 3(1): 48-68; Tomlinson et al., “Quantitative assays for measuring human telomerase activity and DNA binding properties” Methods. 2017 Feb. 1; 114:85-95; Cohen et al., “A sensitive direct human telomerase activity assay” Nature Methods volume 5, pages355-360(2008); Zhen et al., “One-Step High-Throughput Telomerase Activity Measurement of Cell Populations, Single Cells, and Single-Enzyme Complexes” ACS Omega 2020, 5, 38, 24666-24673, each of which is herein incorporated by reference in its entirety for all purposes.

### Genome Modification Agent Delivery

In some instances, a genome modification agent may be delivered to an animal primary cell or animal primary cell line by a variety of delivery techniques. In some cases, a genome modification agent may comprise an agent that can create a genome modification in an animal primary cell or animal primary cell line. For example, a genome modification agent may comprise a CRISPR/Cas9 endonuclease, guide RNA or sleeping beauty transposon. A genome modification agent may also comprise a CRISPR/Cas protein. In some cases, a genome modification agent may comprise an agent that can create a genetic knock-in or knock-out. In some cases, a genome modification agent may comprise the nucleic acid form that encodes any components of the genome modification agent. In some cases, a genome modification agent may comprise the DNA. In some cases, a genome modification agent may comprise the RNA form of the genome modification agent. In some cases, a genome modification agent may comprise the protein form of the genome modification agent. For example, a DNA or RNA (e.g., mRNA) encoding a CRISPR/Cas protein, a CRISPR/Cas9 endonuclease, or a sleeping beauty transposon may be delivered. In another example, the CRISPR/Cas protein or the CRISPR/Cas9 endonuclease may be delivered to an animal primary cell or animal primary cell line. In other cases, the single guide RNA may be delivered to an animal primary cell or animal primary cell line as a DNA or RNA form. In some cases, a genome modification agent may be delivered with a delivery marker agent. For example, a cell may be transfected with a DNA or an RNA alongside a fluorescent protein as a delivery marker agent to distinguish cells with a genome modification agent delivered from those without the genome modification agent delivered. In other cases, a genome modification agent may comprise a delivery marker agent. For example, a genome modification agent may be a fusion protein with a delivery marker agent such as a fluorophore.

In some instances, a knock-in sequence or insertion sequence may be maintained on a genetic construct. A genetic construct, in some cases, may comprise a knock-in sequence or insertion sequence maintained on a plasmid, viral vector, non-viral vector, a linear nucleic acid sequence, a circular nucleic acid, a nucleic acid with a 5′ or a 3′ reducing hydroxyl group.

In some instances, delivering a genetic construct to an animal primary cell or animal primary cell line may comprise transfection. In some cases, transfection may comprise a non-viral or viral transfection. In some cases, a non-viral transfection may comprise a physical transfection or chemical transfection. In some cases, a non-viral transfection may comprise a cell-penetrating peptide (CPP), arginine nanoparticle, membrane fusion, graphene oxide-poly(ethylene glycol)-polyethylenimine (GO-PEG-PEI). In some cases, a non-viral transfection may comprise calcium phosphate, silica, gold, magnetic particles, poly(lactic-co-glycolic acid), poly lactic acid, poly(ethylene imine), chitosan, dendrimer, polymethacrylate, cationic liposome, cationic emulsion, solid lipid nanoparticle, poly-L-lysine, SAP, protamine, needle injection, baslistic DNA injection, electroporation, cell squeezing, sonoporation, photoporation, magnetofection, or hydroporation. In some cases, the choice of the forms (RNA, RNA, or proteins) may influence the delivery methods, as described in Sung et al., “Recent advances in the development of gene delivery systems” Biomaterial Resarch. 2019 Mar. 12; 23:8, which is herein incorporated by reference in its entirety for all purposes.

In some instances, a CRISPR/Cas protein and single guide RNA may be delivered as an RNP particle. In some cases, a CRISPR/Cas9 endonuclease and single guide RNA may be delivered as an RNP particle. In some cases, a CRISPR/Cas protein-single guide RNA or a CRISPR/Cas9 endonuclease-single guide RNA RNP complex may be delivered with lipofectamine. In some cases, lipofectamine comprises a 3:1 mixture of DOSPA (2,3-dioleoyloxy-N-[2(sperminecarboxamido)ethyl]-N,N-dimethyl-1-propaniminium trifluoroacetate) and DOPE (1,2-Dioleoyl-sn-glycero-3-phosphoethanolamine). In some cases, lipofectamine RNAiMax Transfection Reagent (ThermoFisher Scientific; Catalog #13778100, 13778030, 13778075, 13778150, or 13778500) may be used to deliver a CRISPR/Cas protein-single guide RNA RNP, a CRISPR/Cas9 endonuclease-single guide RNA RNP complex, a CRISPR/Cas protein, a CRISPR/Cas9 endonuclease, a single guide RNA, or multiple guide RNAs to an animal primary cell or animal primary cell line. In some cases, the transfection reagents and methods described in Li et al., “Non-viral delivery systems for CRISPR/Cas9-based genome editing: challenges and opportunities” Biomaterials. 2018 July; 171: 207-218; Shi et al., “An improved method for increasing the efficiency of gene transfection and transduction” Int. J. Physiol. Pathophysiol. Pharmacol.; or Cardarelli et al., “The intracellular trafficking mechanism of Lipofectamine-based transfection reagents and its implication for gene delivery” Science Report 2016; 6: 25879, each of which is herein incorporated by reference in its entirety for all purposes, may be used to deliver a CRISPR/Cas protein-single guide RNA RNP, a CRISPR/Cas9 endonuclease-single guide RNA RNP complex, a CRISPR/Cas protein, a CRISPR/Cas9 endonuclease, a single guide RNA, multiple guide RNAs, or any combination thereof to an animal primary cell or animal primary cell line. In some cases, a CRISPR/Cas protein or a CRISPR/Cas9 endonuclease may be linked to a negatively charged protein so that it becomes highly negatively net charged. In some cases, a CRISPR/Cas protein or a CRISPR/Cas9 endonuclease may be linked to a negatively charged protein as a fusion protein. Such a highly negatively charged CRISPR/Cas protein or CRISPR/Cas9 endonuclease may be delivered to an animal primary cell or animal primary cell line using lipofectamine. Such a method may be described in Zuris et al., “Efficient Delivery of Genome-Editing Proteins In Vitro and In Vivo” Nature Biotechnology 2015 Jan; 33(1): 73-80, which is herein incorporated by reference in its entirety for all purposes. The method described herein may be used for any genome modifications described herein and thereof. In some cases, electroporation may be used for transfecting a genome modification agent. In one case, a Lonza electroporation machine (e.g., Nucleofector™ Device) may be used for electroporation. In another case, Lonza electroporation reagents (e.g. Nucleofector™ Kits) may also be used for electroporation. In some cases, any delivery methods described herein may be combined with any other delivery method to increase or improve the efficiency of the delivery.

Producing Meat from Animal Cells

In some instances, meat may be produced by collecting a subset of cells from a master cell line after they reach the desired cell density and forming the collected cells into meat.

In some instances, an animal primary cell may comprise a cell from a pig, cow, chicken, turkey, sheep, goat, fish. In some case, an animal primary cell may comprise a cell from a pig. In some case, an animal primary cell may comprise a cell from a cow. In some case, an animal primary cell may comprise a cell from a chicken. In some case, an animal primary cell may comprise a cell from a turkey. In some case, an animal primary cell may comprise a cell from a sheep. In some case, an animal primary cell may comprise a cell from a goat. In some case, an animal primary cell may comprise a cell from a fish. In some cases, an animal primary cell may comprise a cell of an organism from the kingdom Animalia. In some cases, an animal primary cell may comprise a cell of an organism from the phylum Chordata. In some cases, an animal primary cell may comprise a cell of an organism from the class Mammalia. In some cases, an animal primary cell may comprise a cell of an organism from the order Artiodactyla. In some cases, an animal primary cell may comprise a cell from an organism from the class Actinopterygii. In some cases, an animal primary cell may comprise a cell from an organism from the class Ave. In some instances, an animal primary cell may comprise a cell from a domesticated or wild animal. In some cases, an animal primary cell may comprise a cell from a poultry. A poultry, in some cases, may comprise a chicken, hen, turkey, duck, goose, fowl, squab, pheasant, or pigeon. In some cases, an animal primary cell may also comprise a cell from a buffalo, deer, moose, rabbit, or caribou. In some cases, the choice of an animal for consumption may depend on cultures, religions, or geographical locations. In some instances, an animal cell may comprise a cell with a genome sequence most similar to any organism as described here, when compared to other organisms.

In some instances, an animal primary cell may comprise a cartilage cell, a liver cell, a heart cell, a kidney cell, a bladder cells, or a lung cell. In some cases, an animal primary cell may comprise a cartilage cell. In some cases, an animal primary cell may comprise a liver cell. In some cases, an animal primary cell may comprise a heart cell. In some cases, an animal primary cell may comprise a kidney cell. In some cases, an animal primary cell may comprise a bladder cell. In some cases, an animal primary cell may comprise a lung cell. In some cases, an animal primary cell may comprise a cell from an intestine, joint, skin, membrane, ovary, vas deferen, bone, gill, gizzard, brain, bone marrow, blood, trotter, chitterling, snout, spleen, pancreas, testicle, scrotum, thymus, tripe, reticulum maw, stomach, hom, hoof, or hide.

In some instances, an animal primary cell may comprise a fibroblast cell, a muscle cell, a fat cell, or an endothelial cell. In some cases, an animal primary cell may comprise a fibroblast cell. In some cases, an animal primary cell may comprise a muscle cell. In some cases, an animal primary cell may comprise a fat cell. In some cases, an animal primary cell may comprise an endothelial cell.

In some instances, a muscle cell may be a myogenic cell. In some cases, a myogenic cell may comprise a natively myogenic cell. In some cases, a natively myogenic cell may comprise a myoblast, myocyte, satellite cell, side population cell, muscle derived stem cell, mesenchymal stem cell, myogenic pericyte, or mesoangioblast. In other cases, a myogenic cell may be a non-natively myogenic cell. In some cases, a non-natively myogenic cell may comprise a fibroblast or non-myogenic stem cell. In some cases, a muscle cell may comprise a skeletal muscle cell, cardiac muscle cell, or a smooth muscle cell. In some cases, a skeletal muscle cell may comprise a myoblast, myocyte, and skeletal muscle progenitor cell. In other cases, a skeletal muscle progenitor cell may comprise a myogenic progenitor. In some cases, a myogenic progenitor may comprise a satellite cell, side population cell, muscle derived stem cell, mesenchymal stem cell, myogenic pericyte, tendon cell, or mesoangioblast. In some instances, a liver cell may comprise a hepatocyte.

In some instances, a skin cell may comprise a keratinocyte, melanocyte, Merkel cell, or a Langerhans cell. A skin cell, in some cases, may also comprise a trichocyte cell. In some cases, a keratinocyte may comprise a corneocyte. In some cases, a melanocyte may comprise a Nevus cell. In some instances, a fat cell may comprise an adipocyte cell or lipoblast. A fat cell, in some cases, may also comprise a white fat cell, brown fat cell, or arrow fat cell. In some instances, a cartilage cell may comprise a chondroblast or chondrocyte. In some instances, a bone cell may comprise an osteoblast or osteocyte. In some instances, a fibroblast cell may comprise a fibrocyte cell. In some instances, a heart cell may comprise a cardiomyocyte cell. A heart cell, in some cases, may also comprise a fibroblast. In some instances, a kidney cell may comprise a podocyte cell, mesangial cell, glomerular endothelium cell, Macula Densa cell, tubule epithelium cell, or parietal epithelium cell. A kidney cell, in some cases, may comprise a kidney glomerulus parietal cell, kidney glomerulus podocyte, kidney proximal tubule brush border cell, Loop of Henle thin segment cell, a thick ascending limb cell, a kidney distal tubule cell, a collecting duct principal cell, a collecting duct intercalated cell, or an interstitial kidney cell. In some instances, an endothelial cell may comprise a pulmonary alveolar epithelium cell. In some cases, a lung cell may comprise a pneumocyte or alveolar macrophage. In some cases, a pulmonary alveolar epithelium cell may comprise an alveolar type I (AT1) or type II (AT2) cell.

In some instances, a meat may be an animal primary cell or animal primary cell line grown to a desired density. In some cases, producing a meat may comprising growing a master cell line to a desired density. In some cases, a meat may comprise a cell. In some cases, a meat may comprise more than one type of cells. In some cases, the types and the desired density of animal primary cells or animal primary cell lines may depend on the types of meat being produced. In some cases, a meat may comprise a group of cells from a group of animals described herein and thereof.

### EXAMPLES

These examples are provided for illustrative purposes only and not to limit the scope of the claims provided herein.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

### Example 1: Generation of Pig Primary Cell Lines with High Proliferative Capacity

Disclosed herein are reagents and methods to generate an immortal pig primary cell line with high proliferative capacity and ability for 3D growth.

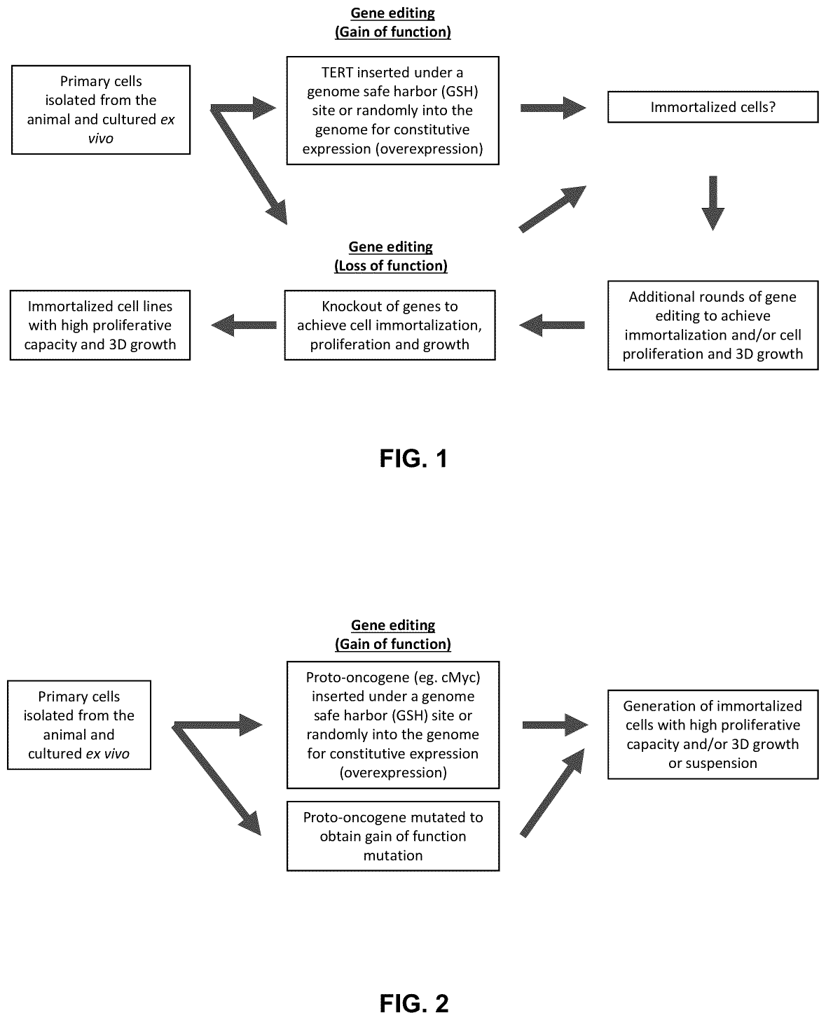

As shown in FIG. 1, primary cells are isolated from an animal and cultured ex vivo. The primary cells are immortalized by either knock-in (overexpressing) porcine TERT (gain of function mutation) or by knocking out (loss of function) porcine genes associated with immortalization or by using both methods simultaneously or independently. The TERT construct is about 3.4 kb in size and targets a genome safe harbor (GSH) and housekeeping gene. TERT construct can also be randomly inserted into the host genome. Once knocked-in, TERT is constitutively expressed to an overexpression level. The constitutive overexpression of TERT will convert the primary cells into immortal primary cells (i.e., primary cell line). Additional or independent round of gene editing (loss of function mutations) of the cells could also achieve immortalization. Knocking out genes in the primary or immortalized primary cell increases the proliferative capacity of the immortal primary cell. These cells can also grow in a 3D manner or suspension.

To generate the knock-out, Cas9 endonuclease coupled with a specific sgRNA sequence (20 nucleotides) is designed to introduce frameshift mutations that can create loss-of-function mutations of the above gene. Mutations in the start codon causes random indels that disrupt or change the reading frame of the gene, resulting in early termination of gene product and no protein production.

Other genes that can be knocked out in the second or third genome modification can comprise any genes described herein and thereof, such as any genes FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A.

One method for generating the porcine TERT knock-in is shown in Example 4.

### Example 2: Generation of Primary Cell Isolated from an Animal with High Proliferative Capacity and 3D or Suspension Growth

Disclosed herein are reagents and methods to generate an immortal pig primary cell line with high proliferative capacity and ability for 3D growth and/or in suspension.

As shown in FIG. 2, primary cells are isolated from an animal and cultured ex vivo. The primary cells are transfected with a proto-oncogene (eg. cMyc) knock-in construct. The knock-in construct targets a housekeeping gene (e.g., Rosa26, pH11, GAPDH, Pifs501, ACTB, AAVS1, HPRT, TBP, HMBS, and CEP112). The knock-in construct can also be inserted at a random genetic location by a sleeping beauty transposon. Once knocked-in or inserted, cMyc is constitutively expressed to an overexpression level. The constitutive overexpression of cMyc increases the proliferative capacity of the primary cell. The knock-in construct can also introduce point mutations to obtain a gain of function mutation. These cells can grow to a desired density for meat production.

Cell lines carrying the cMyc knock-in can also be generated by methods described in Example 4.

### Example 3: Generation of Mutated Cell Lines Using CRISPR/Cas9

Disclosed herein are reagents and methods to generate a loss-of-function mutant animal cell using Cas9 endonuclease and sgRNA.

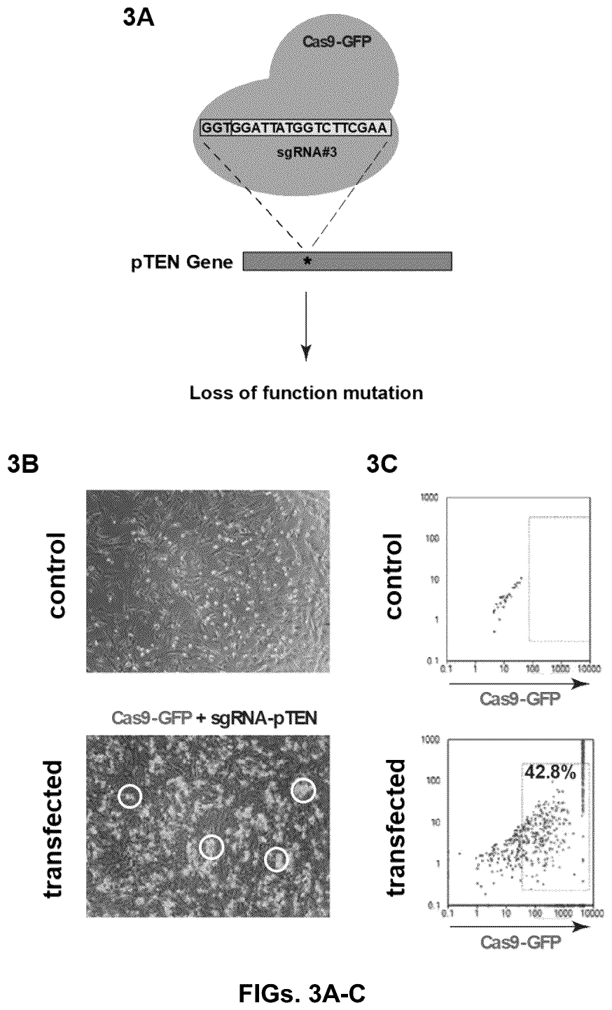

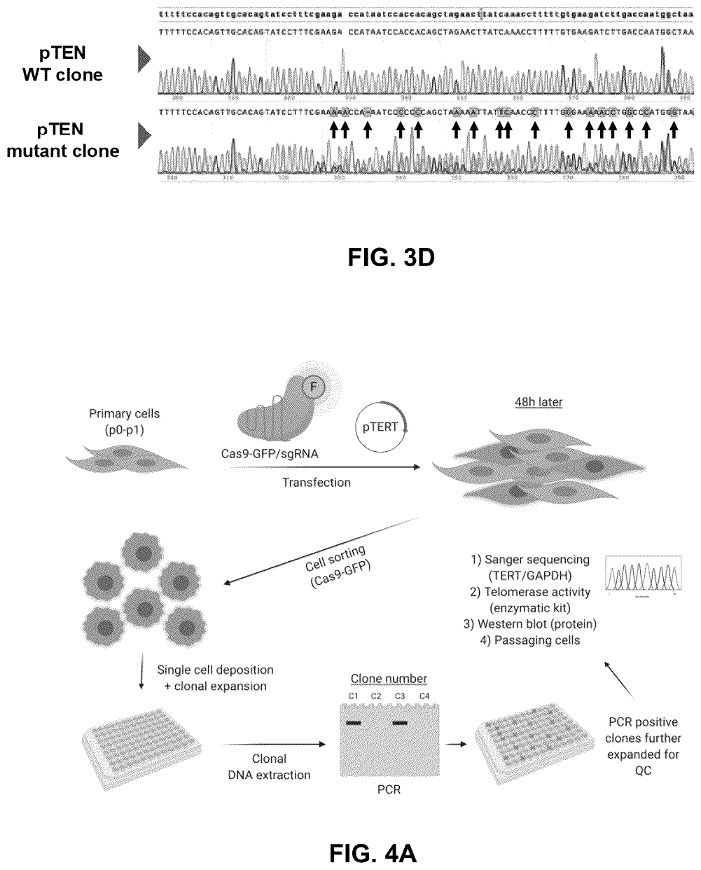

As shown in FIG. 3A, a GFP-tagged Cas9 endonuclease complexed with a sgRNA targeting the pTEN locus can be used to create a loss-of-function mutation in the pTEN locus in the recipient animal cell. To transfect the cells, lipofectamine RNAiMax was mixed with Cas9 endonuclease/sgRNA RNP complex such that a liposome was formed encapsulating the RNP complex. The liposome containing the RNP complex fuses with the cell membrane of the host for RNP delivery. Similar results are obtained with nucleofection using Lonza 4D-Nucleofector™ Because the Cas9 protein was GFP tagged, cells successfully transfected were GFP positive, as shown in FIG. 3B. Single GFP-positive cells were then sorted by flow cytometry using a single cell sorter and dispenser (Namocell), as shown in FIG. 3C, and deposited in 96-well plates for clonal expansion. The expanded cell lines were genotypically identified for mutations using sequencing, such as Sanger sequencing, as illustrated in FIG. 3D. Only mutations that introduced a frameshift mutation were selected for further analysis.

Similar strategy can also be used to create a loss-of-function mutation in a gene described herein and thereof, such as any genes selected from the group consisting of. FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A. When creating indel mutation in the target locus, the indel efficiency generated can also be determined by ICE CRISPR Analysis Tool (Synthego), SnapGene or TOPO cloning.

### Example 4: Generating Pig TERT Knocked-In Cells

Disclosed herein are reagents and methods to generate an animal primary cell line with a TERT knock-in construct.

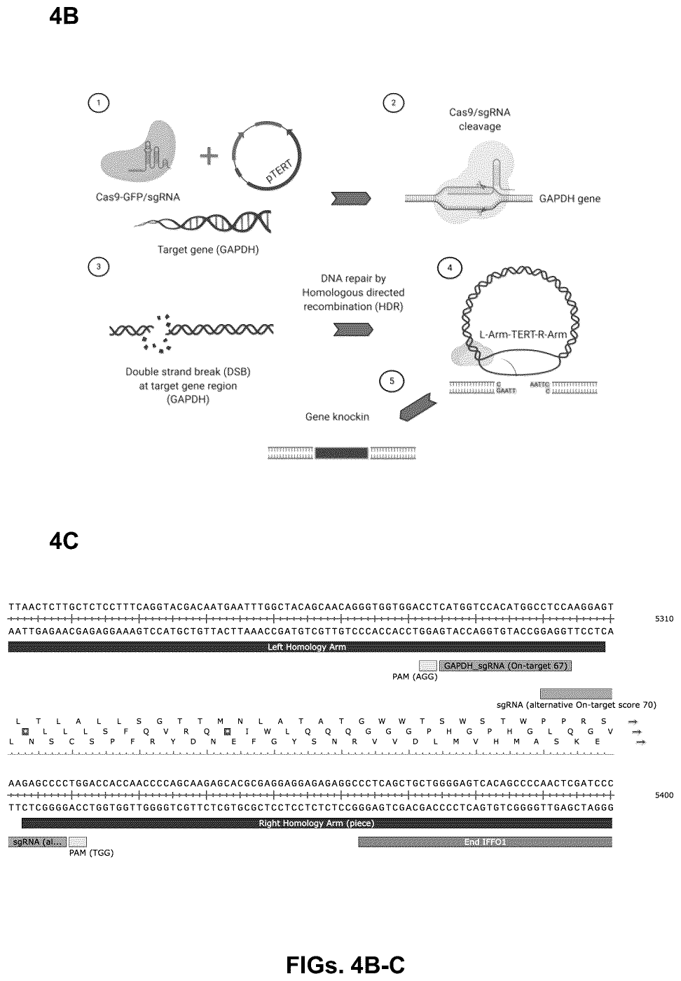

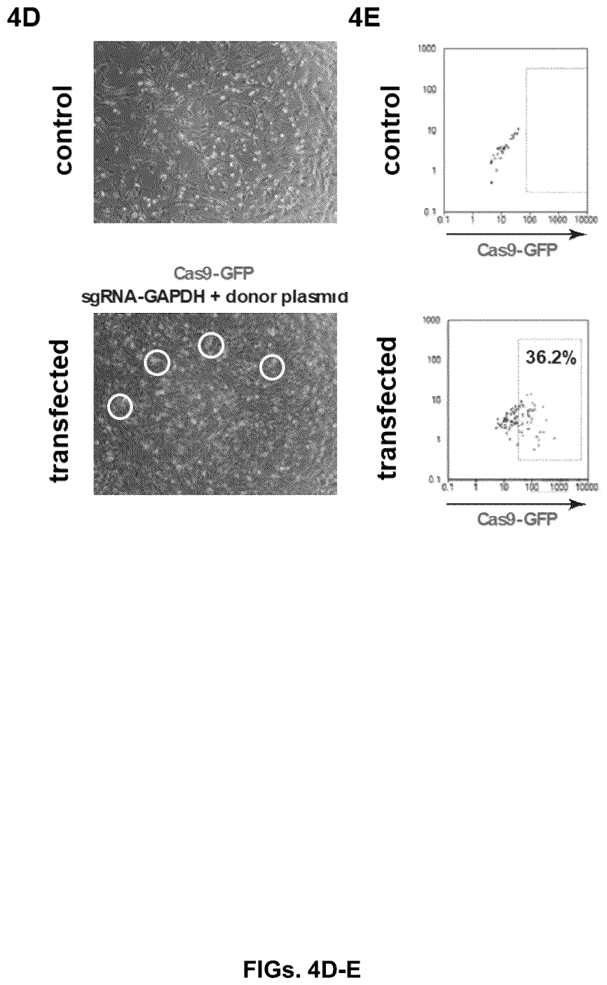

As shown in FIG. 4A, primary cells are transfected with a Cas9-GFP/sgRNA RNP complex and a TERT knock-in construct using electroporation/nucleofection techniques. Successfully transfected cells, positive for GFP, are sorted (Namocell) and deposited as single cell in multi-well plates for clonal expansion post transfection. Once clonally expanded, DNA is extracted from each clone for PCR amplification of the knock-in construct. The PCR-positive clones are tested in additional tests to confirm successful knock-in of TERT with constitutive overexpression: 1, sanger sequencing of TERT inserted at the GAPDH locus; 2, measuring telomerase activity using enzymatic kit; 3, measuring TERT's protein expression level; 4, passaging the clonal cells and; 5, gene expression analysis by RTqPCR.

FIG. 4B depicts a schematic of knocking-in the porcine TERT into the GAPDH locus by using Cas9-GFP/sgRNA RNP complex and a donor plasmid carrying the porcine TERT construct. A Cas9 endonuclease/sgRNA RNP complex targeting the GAPDH gene and TERT knock-in construct with two homology arm sequences to the GAPDH gene are co-transfected to an animal primary cell. Once transfected, the Cas9/sgRNA RNP complex mediates a cleavage at the GAPDH gene based on the sequence of the sgRNAs. Exemplary sgRNA sequences targeting the GAPDH locus are shown in FIG. 4C. A double strand break at the GAPDH gene is created. Using the homology arms (L-Arm and R-Arm) of the knock-in construct, the TERT is inserted at the double strand break using endogenous repair mechanisms. HDR repair results in either a homozygous (both allele) or heterozygous (one allele) insertion of TERT into the GAPDH locus.

Porcine cells were co-transfected with the Cas9-GFP/sgRNA RNP complex and the donor plasmid carrying the porcine TERT knock-in construct, using nucleofection. Successfully transfected single GFP-positive cells were then sorted by flow cytometry using a single cell sorter and dispenser (Namocell) as shown in FIGS. 4D and 4E, and deposited in 96-well plates for clonal expansion. Cells transfected with TERT knock-in plasmid can be selected by treating the cells with gentamycin (G418).

Donor DNA (plasmid or ssDNA) can carry other knock-in transgenes or constructs, such as those described in this disclosure, including but not limited cMyc. Other target loci may also be used, such as those housekeeping genes described in this disclosure. The sgRNA sequence and the homology arm sequence will depend on the gene being targeted. Exemplary sgRNA can be found in Table 1 listed below:

TABLE 1
Exemplary sgRNA sequences

| sgRNA | Sequence | SEQ ID NO. |
|---|---|---|
| GAPDH_sgRNA | CTCCAAGGAGTAAGAGCCCC | SEQ ID NO. 2 |
| #1 | | |
| GAPDH_sgRNA | CAACAGGGTGGTGGACCTCA | SEQ ID NO. 3 |
| #2 | | |
| pTEN_Exon5 | GGUGGAUUAUGGUCUUCGAA | SEQ ID NO. 4 |
| RB1_Exon2 | TGACATAATGTAGTAAATC | SEQ ID NO. 5 |
| ATRX_Exon2_ | TGAAAGTAAGTTGAATACAC | SEQ ID NO. 6 |
| 1 | | |

Exemplary plasmid DNA can be found in Table 2 listed below:

TABLE 2
Exemplary donor DNA sequences

| Plasmid | Sequence | SEQ ID NO. |
|---|---|---|
| Plasmid | gacggatcgggagatctcccgatcccctatggtgcactctcagtacaatctgctctgatg | SEQ ID NO. 7 |
| containing | ccgcatagttaagccagtatctgctccctgcttgtgtgttggaggtcgctgagtagtgcgc | |
| pTERT #1 | gagcaaaatttaagctacaacaaggcaaggcttgaccgacaattgcatgaagaatctgc | |
| | ttagggttaggcgttttgcgctgcttcgcgatgtacgggccagatatacgcgttgacattg | |
| | attattgactagttattaatagtaatcaattacggggtcattagttcatagcccatatatggag | |
| | ttccgcgttacataacttacggtaaatggcccgcctggctgaccgcccaacgacccccg | |
| | cccattgacgtcaataatgacgtatgttcccatagtaacgccaatagggactttccattgac | |
| | gtcaatgggtggagtatttacggtaaactgcccacttggcagtacatcaagtgtatcatat | |
| | gccaagtacgccccctattgacgtcaatgacggtaaatggcccgcctggcattatgccc | |
| | agtacatgaccttatgggactttcctacttggcagtacatctacgtattagtcatcgctatta | |
| | ccatggtgatgcggttttggcagtacatcaatgggcgtggatagcggtttgactcacggg | |
| | gatttccaagtctccaccccattgacgtcaatgggagtttgttttggcaccaaaatcaacg | |
| | ggactttccaaaatgtcgtaacaactccgccccattgacgcaaatgggcggtaggcgtg | |
| | tacggtgggaggtctatataagcagagctctctggctaactagagaacccactgcttact | |
| | ggcttatcgaaattaatacgactcactatagggagacccaagctggctagcgtttaaactt | |
| | aagcttTGGAATCTAATCCTAATGAAACTGGCGAGTATT | |
| | TATTGAACGCTTTGGTGTCACTGTTTCCAGCAACTTTT | |
| | CGGTGGTGGCTCTGGTCCCTGTTGTGTGGTGTGCACT | |
| | GGTGATCATTGCACAGACACACAGGGTTACAAGCAA | |
| | CTTGCCCAAAGTAATGGCACCTCAAGTAGCAGAACC | |
| | AGGGCCTCCATCCAGGTTGTCAGTGCTGTCTCTGCTT | |
| | CTTCCCACTAAGCCTGTGCGCATTCGCAGGTGACAGA | |
| | AGACATGGAAATAAAACCACCCCAACCTTTCCCTTGG | |
| | GCTAACCTACTGTAACTTCTCTGAACAATCTCTGAGC | |
| | TGGCACTGGCACAGCTGTACCACAGATGGCAATCTCT | |
| | TGGGCACTGAGGCTCACAGCTGTGACAGGGCAGAGC | |
| | TGGAATCCACTCCTGGCAGTCAGGCCGTAGCCAAATG | |
| | GACTTCCTGTGCCAGTGCCCCCCTCCAGGGAGCTGCA | |
| | GACAGAATGGTGTCTTGGGTGCAAGCAGCCAGGCCA | |
| | TTCCTGGGTTCAGCCACGGCAGTCTGGAGCTGTAGCA | |
| | CCCAGAGGCCTCACTAAGGCAACACGAGGACAGGTG | |
| | GCTCTTCAGAGGAGCCCAGGGTGGAGTTTCCTCAGCA | |
| | AGCTCTAGTTCTCCTTGGCCCCAGCCCGGCTTCCCTCC | |
| | TCTTTATCCCAGGCTCTAGAGTGAGAACGACAGGAG | |
| | GCTGGGTACAGTGTACTTTATTGATGGTACATGACGA | |
| | GGCAGGTCTCCCTAAGCCCCTCCCCTTCTTCCGGGGG | |
| | TCTGGGATGGAAACTGGAAGTCAGGAGATGCTCGGT | |
| | GTGTTGGGGGATCGAGTTGGGGCTGTGACTCCCCAGC | |
| | AGCTGAGGGCCTCTCTCCTCCTCGCGTGCTCTTGCTG | |
| | GGGTTGGTGGTCGAGGGGCTCtcagtccaggatggtccggaagtct | |
| | gtagtcagggctgggtcggcggcggcctcgagggcggccagtgtggccccagggag | |
| | ccgctggcacaatcgctctctggctgcccggagcggccccagaagacagctgtaggta | |
| | acgcggtgacgaaccagcttgagcaggaaggcgtggaggcagagccactctgcggc | |
| | ttcagagggaaacgggccggaggcgcccctggctcccagggacatccctgcgtttctg | |
| | gctttcaggagcgagtagcagcaggacgcggtgtcggagacaagccggaggaaaaa | |
| | tgaggggttcctcgcaagcggctgacggaagggcagctgcagcacacacgcgtgga | |
| | acctgtaggcctgcagcaggaagatcttgtaaacgttggcgagcaccgtcggaagact | |
| | gttcacctgcaggtccagaaggatcccgtggcactttagccgcaagaccgccaagagc | |
| | ttgcggcgcatgttcctcccgggcttgaagccctggttgaaggtgagactcgctctgatc | |
| | gaggtccgggcgtaactggcatagtcgcagtgcacctccagcgtgcgggtgtccagca | |
| | gcagcccgcaccaggggaacaggcagtgtgccggcagctgcagtggggccgggcc | |
| | gccccgggcgccgtcctccacagggaagttcacggccgtcttccgcaagttggccagg | |
| | cagccgtactcgggcacgccgcggaccagggtcctgagaaaggctttggctcgtgtca | |
| | ggtgaggggtcaccagcaggaagtcgtccaccaagcgcaggagcaccccgtcacgc | |
| | tggatcccggggaagagtctgttttccatgtccccgtagcacaggctgcagagcagcgt | |
| | ggacagaatggagccctgtgggatcccctgacactggacgtaggacctgcccccgatc | |
| | cggatgacgtggtttcgcagcaggctcaggaaaagctccaggagacggctgccggcc | |
| | tcgttcagagagcagctctgctcgatgaccacggcgtccctcagcgggccggttgcctg | |
| | caggtgtgccacaaactgtctcatgtaaggctggaggtctgcgaaggtggacacgtgg | |
| | gtttggaaggacttgcgcacgtgcccgcgggcggtcctccggagcacggcgcactgg | |
| | cgcacgcagtacgtgctctcgtggggccggatcacgttggcgaccacctccagcagcc | |
| | tgtcctgagggagggcgtcgtaggcccccgtcacgtccaccttgacaaagtacagcgg | |
| | gggggccgggtcctgggcccgcaggggcagcacaaaggcccgccaggccctgtgg | |
| | atgtcacccacgcccagcacggaggcccccaggaggcgcgggcgccgcgcccgct | |
| | cgtagttcagcacagcaaacagcgtcttgacctgccccgtgagacgccggaccttcttgt | |
| | ctccggggcctgtcctggctcgcacgacgttcgccatgttcacgatgggccgcagccc | |
| | gtcgggtttggggacgaagcggagcttggaggtcagtacagcgggcctggcctcccg | |
| | gcgtcgcctgatctctgcttccgacagttctcgaagccgcacacgatctaagtgttgcctg | |
| | atgcctgcgctctgcagccggctccagatgcgcttccggaagaagaagagccggttctt | |
| | ctgaaacgtggtctccgtgacataaaaaaacgacctgagcagctcgaccacgtacgtgc | |
| | ccatcaaccagcgcaggaacttggccagaatggcctctctcagacggtgctcggcggc | |
| | ctggacatggcgagcgtccgggctccggcgcagccagatgcagtcttgcactttcatctt | |
| | ccacatcagctcctgcagcgagagcctggcgtgcttccccagggagacgagcttcttca | |
| | cattcttcagaaagcgccgcttgttgtgcggggagccccagaggcccgggggcacca | |
| | gccggtgaagacaggcccgcaggaggcggtacacctgccaggggctgctgtgctgg | |
| | cggagcagctggactaggcgcctcgaggccgagtccccctcggcggccgctgccag | |
| | ccgctctggagcgagggggcccactttgtggtcgcacacctgaccgccggaccccga | |
| | gagggtcgccgcagcccgcaacgggcagtgcgccctgagaagcgccttgtaggggc | |
| | accgcgcgtggttcttaagcagctcccggaacaggggcctcatccgccagtagcgtgc | |
| | gggcacgcggcgcgcccctggctgcccgggctttgaggccagaaagatgacctccac | |
| | gagcctcccggcccccgccaggctgggcggcagggagcagagcaggaacgagcg | |
| | gcgcagcccttccttgctccccgagcagtagaggaagcgcttggtctcggcagcgcac | |
| | acatgaggactgggcttgccctggaggtgtggctgccaccaggatggggggcaggta | |
| | ccccgcgcaccgcctgcttgaggggaaaggtgccgggttccgggcgcctcaccctcc | |
| | cgagacttggctttcgcagcggctctggtaggtgtcatcacgggggggtcgctgtcgct | |
| | cggcccaggcgccctgccctggtgggcccgggacgcctgcccttcgggaccctgttc | |
| | aggctccgggacgtgcctgggcctcttggctggaagtggatgtccccccgctctgcccc | |
| | gacgtcgcctcacaccttgcgccctctgctcctggggtcgcccggcttccccattgcaaa | |
| | cggggcgcgtgagtccgaggccagtcggggtctggcccttgtgtcgcatgggccgag | |
| | cctccgctgcggtgtagaggtcatagagtggcggcccgcacacctggtaggcgcaact | |
| | cgggggcaccagcaggtacagcgcgcagcgcgccaacaggtgggtgagcacgtcgt | |
| | cgcccacgcggcgcagcagcagcccccacgcgccgctcccgcgcagtgtgtcggtc | |
| | acggtgttgggcaggtagctgcgcacgctggtcgtgaaggccacgggcggcccgcc | |
| | gcgagccccgtccagcagcgcgaagccaaaggccagcacgttcctcgcgccgcgct | |
| | cgcagagcctctgcacgaccctggccaccagctccttcaggcaggacacctggcgga | |
| | aggacggggaggcaggaggcggctgcgcgtcccagggcacgcacaccaggcactg | |
| | cgccaccagcgcgcggtaggccgccgggtccccgcgccgaacaagccgccggccc | |
| | tcagggcccaggcgccgcacgaaggtggccagcggcagcacctgcctgtagcggtc | |
| | ccggagcagggagcgcacggcccggcaccggggcgcgcgcggcatcgccgagag | |
| | gagggagGGGGCCGGGGTTCTCCTCCACGTCGCCGGCCT | |
| | GCTTCAGCAGGCTGAAGTTGGTGGCTCCGCTTCCCTC | |
| | CTTGGAGGCCATGTGGACCATGAGGTCCACCACCCTG | |
| | TTGCTGTAGCCAAATTCATTGTCGTACCTGAAAGGAG | |
| | AGCAAGAGTTAAGTGTGGTTTTCCCGAGCAGCCACCA | |
| | GGGGGCACCAGACTTGATCACATTCTGAAGCATCATC | |
| | CCTCCCCACCCGCCACATCCCCCAGCACCCTACCAGG | |
| | AAATGAGCTTGACGAAGTGGTCGTTGAGGGCAATGC | |
| | CAGCCCCAGCATCAAAAGTGGAAGAGTGAGTGTCAC | |
| | TGTTGAAGTCACAGGACACAACCTGGGGAAGGAAGA | |
| | GGGCCAATTACGGAACCCAGTCTTGGTCAGTGGGGTC | |
| | GTGTGGACACCAAGTTCCCTGACCTATCAGTACCTGG | |
| | TCCTCAGTGTAGCCCAGGATGCCCTTGAGGGGGCCCT | |
| | CGGACGCCTGCTTCACCACCTTCTTGATGTCATCATA | |
| | TTTTGCCTGATGAGGTAAAAACCGATGGTTATTCAAG | |
| | TACCAATCCTACAACCCCTCAGCCCCCCAAAGCTGTT | |
| | CCAACCCAAACCTACAGGTTTCTCCAGGCGGCAGGTC | |
| | AGATCCACAACCGACACGTTGGGGGTGGGGACACGG | |
| | AAGGCCATGCCAGTGAGCTTCCTGGGGACACGAGGA | |
| | AGCAAGCAGAGTCAGCACCACCCCTGCTGGTGCCCC | |
| | AGACACCCAGGGCAGGAGAGCAGCAAAGAGCCTCAC | |
| | CCGTTGAGCTCAGGGATGACCTTGCCCACAGCCTTGG | |
| | CAGCGCCGGTAGAAGCAGGGATGATGTTCTGGGCAG | |
| | CCCCTCGGCCATCACGCCACAGCTTCCCAGACGGGCC | |
| | ATCCACAGTCTTCTGGGTGGCAGTGATGGCATGGACC | |
| | GTGGTCTGGAGACAAGGAGGGGGAGTGAAGGCCCTG | |
| | CCTAGCGCTTGTGACGCCggtaccgagctcggatccactagtccagtgt | |
| | ggtggaattctgcagatatccagcacagtggcggccgctcgagtctagagggcccgttt | |
| | aaacccgctgatcagcctcgactgtgccttctagttgccagccatctgttgtttgcccctcc | |
| | cccgtgccttccttgaccctggaaggtgccactcccactgtcctttcctaataaaatgagg | |
| | aaattgcatcgcattgtctgagtaggtgtcattctattctggggggtggggggggcagg | |
| | acagcaagggggaggattgggaagacaatagcaggcatgctggggatgcggtgggc | |
| | tctatggcttctgaggcggaaagaaccagctggggctctagggggtatccccacgcgc | |
| | cctgtagcggcgcattaagcgcggcgggtgtggtggttacgcgcagcgtgaccgct | |
| Plasmid | tatacagttgaagtcggaagtttacatacacttaagttggagtcattaaaactcgtttttcaa | SEQ ID NO. 8 |
| containing | ctactccacaaatttcttgttaacaaacaatagttttggcaagtcagttaggacatctactttg | |
| cMyc | tgcatgacacaagtcatttttccaacaattgtttacagacagattatttcacttataattcact | |
| | gtatcacaattccagtgggtcagaagtttacatacactaagttgactgtgcctttaaacagctt | |
| | ggaaaattccagaaaatgatgtcatggctttagaagctagatctggccatctagagggca | |
| | gcatcctcctgctgagagccgggggagggccaggcccacgtcccgagagcaagcgc | |
| | gaggagacggaggaggtgacccttccctcccccggggcccggtggtgaggggaggt | |
| | ctctcttttctgtcgcacccttaccttgtcccaggcctgggcccgggctgcggcgcacgg | |
| | cactcccggtaggcagcaggactcgagttaggcccagcgcggcgccacggcgtttcct | |
| | ggccgggaatggcccgtgcccgtgaggtgggggtggggggcaaaaaggcggagcg | |
| | agccaaaggcggtgaggggggagggccagggaaggaggggggggccggcactac | |
| | tgtgttggcggactggcgggactggggctgcgtgagtctctgagcgcaggcgggcgg | |
| | cggccgcccctcccccggcggcggcggggggggggggcggcagcagctc | |
| | actcagcccgctgcccgagcggaaacgccactgaccgcacggggattcccagcgcc | |
| | ggcgccaggggcacccgggacacgccccctcccgccgcgccattggcccctccgcc | |
| | caccgtctcgcacccattggccagctccccgccaatcagcggaagccgccggggccg | |
| | cctagagaagaggctgtgctctggggctccggctcctcagagagcctcggctaggcca | |
| | ccatgcccctcaacgtcagcttcaccaacaggaactatgacctcgactacgactcggtg | |
| | cagccttatttctactgcgacgaggaggagaacttctaccagcagcagcagcagagcg | |
| | agctgcagccgccggcacccagcgaggatatctggaagaaattcgagttgctgcccac | |
| | cccgcccctgtccccgagccgccgctccgggctctgctcgccctcgtacgtcgcggtc | |
| | gcgtccttctcccccaggggagacgatgacggcggcggcggcagcttttccacggcc | |
| | gaccagttggagatggtgaccgagctgctgggaggagacatggtgaaccagagcttca | |
| | tctgcgaccccgacgacgagaccttcatcaaaaacattatcatccaggactgtatgtgga | |
| | gcggcttctcagccgccgccaagctcgtgtcagagaagctggcctcctaccaggctgc | |
| | gcgcaaagacagcggcagcccgattcccgcccgcgggcacggcggctactccacct | |
| | ccagcttgtacctgcaggacctgagcgccgccgcctccgaatgcatcgacccctcggt | |
| | ggtcttcccctacccgctcaacgacagcagctcgcccaagccctgcgcctcccccgact | |
| | ccaccgccttctccccgtcctcggactctctgctctcctccgccgagtcctccccacggg | |
| | ccagtcccgaacccttggctctccacgaggagacaccacccaccaccagcagcgact | |
| | ctgaggaagaacaagaggatgaggaagaaattgatgttgtttctgtggaaaagaggca | |
| | gccccctgccaaaaggtcggaatcggggtcaccctctgctggaggccacagcaaacct | |
| | cctcacagccccctggtccttaagagatgccatgtgtccacgcaccagcacaattatgca | |
| | gcgcccccctccaccaggaaggactatccctctgccaagagggctaagttggacagtg | |
| | gcagggtcctgaaacagatcagcaacaaccgaaaatgtgccagccccaggtcctcgg | |
| | acacggaggagaatgacaagaggcggacacacaatgtcttggaacgccagaggaga | |
| | aacgagctgaaacgcagcttttttgcccttcgagaccagatcccagagttggagaacaat | |
| | gaaaaagcccccaaggtagttatccttaaaaaagccacagcttacatcctgtctgtccaa | |
| | gcagaggagcaaaagctcgtgtcagaaaaagacgtgctgcggaagaggcgagaaca | |
| | gttgaaactcaaacttgaacagctacggaactcttgcccataaacccagctttcttgtaca | |
| | aagtggctgtgccttctagttgccagccatctgttgtttgcccctcccccgtgccttccttga | |
| | ccctggaaggtgccactcccactgtcctttcctaataaaatgaggaaattgcatcgcattg | |
| | tctgagtaggtgtcattctattctggggggtggggggggcaggacagcaagggggag | |
| | gattgggaagacaatagcaggcatgctggggatgcggtgggctctatggaagcttgtg | |
| | gaaggctactcgaaatgtttgacccaagttaaacaatttaaaggcaatgctaccaaatact | |
| | aattgagtgtatgtaaacttctgacccactgggaatgtgatgaaagaaataaaagctgaa | |
| | atgaatcattctctctactattattctgatatttcacattcttaaaataaagtggtgatcctaa | |
| | ctgacctaagacagggaatttttactaggattaaatgtcaggaattgtgaaaaagtgagttta | |
| | aatgtatttggctaaggtgtatgtaaacttccgacttcaactg | |
| Plasmid | tatacagttgaagtcggaagtttacatacacttaagttggagtcattaaaactcgtttttcaa | SEQ ID NO. 9 |
| containing | ctactccacaaatttcttgttaacaaacaatagttttggcaagtcagttaggacatctactttg | |
| pTERT #2 | tgcatgacacaagtcatttttccaacaattgtttacagacagattatttcacttataattcact | |
| | gtatcacaattccagtgggtcagaagtttacatacactaagttgactgtgcctttaaacagctt | |
| | ggaaaattccagaaaatgatgtcatggctttagaagctagatctggccatctagactgga | |
| | gtaccacgtggtgtgtagaacgaacttagataaaagaactgaatggaaaaagccaaga | |
| | ggtcagcgcaggagcctgttcgccaggctgttggtcagcaaactgagccactcgcgga | |
| | ttctcggccctgaagaccgccccgaggcccctgccgccgcggttccccggccttttacg | |
| | cctccctctccagccggaaggggcgcggacggcaagggcggtggagaagccccgg | |
| | cagaggaagcggctccaggggttagtccttaacctgcgagaccgcaagactgaggac | |
| | cggtggggtactgccgggtaagggctcccatctcggtccaactttgcagttaagctacg | |
| | accggttcagggaagattctggacgcctccccgccgccccaccccccagctgtgcgga | |
| | gagtaattcatacaaaaggaggactctcctcagccagggaaatcccagggaccgtcga | |
| | taaactcccactaaacctagaaccgagtgagcgctcgaccccgcctcccacccaccag | |
| | cagtcgtcatcctcctggttgagaggagcatgcgccgggcgccgtgtgctcgtcagtgg | |
| | gctgaacgcacatcgcccacggtccccgaagatggggggaggggacggcggtgga | |
| | accggtgccgggtggaggtggcgcggggtaaactgggaaagtggtgtcgtgtgctgg | |
| | ctccgcccttttccccgaggggggggaggaccatatataagcgccgtggtccccgcg | |
| | aacgttctttttcgcaacgggtttgccgccaggacacaggtgagtacgggtgtggcctcc | |
| | gtccgcatggcctccgccggtggccacggccttagcgtgcctcccggccccccgcgc | |
| | gtagagggctctgcgccctggtcctgattccgagctgcgggcggggggaggtggaga | |
| | actcgaggccctccgctctcgcggttccctaccgcgtgcccggtggcggcctgctggg | |
| | gcgccgtggccgccgcgtgcgatccgcgccttcgcgcccgccccgtctgtcttcgcgc | |
| | tcgagttcggccggcagccgcgtgttactgccagggcggcccagccgaggccgccca | |
| | aggtggcctccgtgcccggcggtcccggggcagacggccgcggcggggggggc | |
| | gggcggtcggagacccagcgccccgcccggggggctgcgagcgcggaaagatg | |
| | gccgctcccggtctcaagatggaggacgcggacggcgggcggcgcacaaagggcc | |
| | gggcggtgctgcccgcgccgcttcccgggactcgcggcccggggggggaggggct | |
| | gggcgccggggcggccccgttcgctgggtgtaggccgggcagggcccgcagggcg | |
| | ccggtgctaatgctccctggagtttgcccttggctgttggggttgtgcctgagtctccggt | |
| | cgtcgggacagtagtttaaagaaggtttttgtcgtcttaggtgtcgtgaaagccatcgcta | |
| | aaagctaagcaaagccaccatgccgcgcgcgccccggtgccgggccgtgcgctccct | |
| | gctccgggaccgctacaggcaggtgctgccgctggccaccttcgtgcggcgcctggg | |
| | ccctgagggccggcggcttgttcggcgcggggacccggcggcctaccgcgcgctgg | |
| | tggcgcagtgcctggtgtgcgtgccctgggacgcgcagccgcctcctgcctccccgtc | |
| | cttccgccaggtgtcctgcctgaaggagctggtggccagggtcgtgcagaggctctgc | |
| | gagcgcggcgcgaggaacgtgctggcctttggcttcgcgctgctggacggggctcgc | |
| | ggcgggccgcccgtggccttcacgaccagcgtgcgcagctacctgcccaacaccgtg | |
| | accgacacactgcgcgggagcggcgcgtgggggctgctgctgcgccgcgtgggcga | |
| | cgacgtgctcacccacctgttggcgcgctgcgcgctgtacctgctggtgcccccgagtt | |
| | gcgcctaccaggtgtgcgggccgccactctatgacctctacaccgcagcggaggctcg | |
| | gcccatgcgacacaagggccagaccccgactggcctcggactcacgcgccccgtttg | |
| | caatggggaagccgggcgaccccaggagcagagggcgcaaggtgtgaggcgacgt | |
| | cggggcagagcggggggacatccacttccagccaagaggcccaggcacgtcccgg | |
| | agcctgaacagggtcccgaagggcaggcgtcccgggcccaccagggcagggcgcc | |
| | tgggccgagcgacagcgacccccccgtgatgacacctaccagagccgctgcgaaag | |
| | ccaagtctcgggagggtgaggcgcccggaacccggcacctttcccctcaagcaggcg | |
| | gtgcgcggggtacctgccccccatcctggtggcagccacacctccagggcaagccca | |
| | gtcctcatgtgtgcgctgccgagaccaagcgcttcctctactgctcggggagcaaggaa | |
| | gggctgcgccgctcgttcctgctctgctccctgccgcccagcctggcgggggccggga | |
| | ggctcgtggaggtcatctttctggcctcaaagcccgggcagccaggggcgcgccgcgt | |
| | gcccgcacgctactggcggatgaggcccctgttccgggagctgcttaagaaccacgcg | |
| | cggtgcccctacaaggcgcttctcagggcgcactgcccgttgcgggctgcggcgacc | |
| | ctctcggggtccggcggtcaggtgtgcgaccacaaagtgggccccctcgctccagag | |
| | cggctggcagcggccgccgagggggactcggcctcgaggcgcctagtccagctgct | |
| | ccgccagcacagcagcccctggcaggtgtaccgcctcctgcgggcctgtcttcaccgg | |
| | ctggtgcccccgggcctctggggctccccgcacaacaagcggcgctttctgaagaatg | |
| | tgaagaagctcgtctccctggggaagcacgccaggctctcgctgcaggagctgatgtg | |
| | gaagatgaaagtgcaagactgcatctggctgcgccggagcccggacgctcgccatgtc | |
| | caggccgccgagcaccgtctgagagaggccattctggccaagttcctgcgctggttgat | |
| | gggcacgtacgtggtcgagctgctcaggtcgtttttttatgtcacggagaccacgtttcag | |
| | aagaaccggctcttcttcttccggaagcgcatctggagccggctgcagagcgcaggca | |
| | tcaggcaacacttagatcgtgtgcggcttcgagaactgtcggaagcagagatcaggcg | |
| | acgccgggaggccaggcccgctgtactgacctccaagctccgcttcgtccccaaaccc | |
| | gacgggctgcggcccatcgtgaacatggcgaacgtcgtgcgagccaggacaggccc | |
| | cggagacaagaaggtccggcgtctcacggggcaggtcaagacgctgtttgctgtgctg | |
| | aactacgagcgggcgcggcgcccgcgcctcctgggggcctccgtgctgggcgtggg | |
| | tgacatccacagggcctggcgggcctttgtgctgcccctgcgggcccaggacccggc | |
| | ccccccgctgtactttgtcaaggtggacgtgacgggggcctacgacgccctccctcag | |
| | gacaggctgctggaggtggtcgccaacgtgatccggccccacgagagcacgtactgc | |
| | gtgcgccagtgcgccgtgctccggaggaccgcccgcgggcacgtgcgcaagtccttc | |
| | caaacccacgtgtccaccttcgcagacctccagccttacatgagacagtttgtggcacac | |
| | ctgcaggcaaccggcccgctgagggacgccgtggtcatcgagcagagctgctctctg | |
| | aacgaggccggcagccgtctcctggagcttttcctgagcctgctgcgaaaccacgtcat | |
| | ccggatcgggggcaggtcctacgtccagtgtcaggggatcccacagggctccattctgt | |
| | ccacgctgctctgcagcctgtgctacggggacatggaaaacagactcttccccgggatc | |
| | cagcgtgacggggtgctcctgcgcttggtggacgacttcctgctggtgacccctcacct | |
| | gacacgagccaaagcctttctcaggaccctggtccgcggcgtgcccgagtacggctgc | |
| | ctggccaacttgcggaagacggccgtgaacttccctgtggaggacggcgcccggggc | |
| | ggcccggccccactgcagctgccggcacactgcctgttcccctggtgcgggctgctgc | |
| | tggacacccgcacgctggaggtgcactgcgactatgccagttacgcccggacctcgat | |
| | cagagcgagtctcaccttcaaccagggcttcaagcccgggaggaacatgcgccgcaa | |
| | gctcttggcggtcttgcggctaaagtgccacgggatccttctggacctgcaggtgaaca | |
| | gtcttccgacggtgctcgccaacgtttacaagatcttcctgctgcaggcctacaggttcca | |
| | cgcgtgtgtgctgcagctgcccttccgtcagccgcttgcgaggaacccctcatttttcctc | |
| | cggcttgtctccgacaccgcgtcctgctgctactcgctcctgaaagccagaaacgcagg | |
| | gatgtccctgggagccaggggcgcctccggcccgtttccctctgaagccgcagagtgg | |
| | ctctgcctccacgccttcctgctcaagctggttcgtcaccgcgttacctacagctgtcttct | |
| | ggggccgctccgggcagccagagagcgattgtgccagcggctccctggggccacac | |
| | tggccgccctcgaggccgccgccgacccagccctgactacagacttccggaccatcct | |
| | ggactgaacccagctttcttgtacaaagtggctgtgccttctagttgccagccatctgttgtt | |
| | tgcccctcccccgtgccttccttgaccctggaaggtgccactcccactgtcctttcctaata | |
| | aaatgaggaaattgcatcgcattgtctgagtaggtgtcattctattctggggggtggggtg | |
| | gggcaggacagcaagggggaggattgggaagacaatagcaggcatgctggggatgc | |
| | ggtgggctctatggaagcttgtggaaggctactcgaaatgtttgacccaagttaaacaatt | |
| | taaaggcaatgctaccaaatactaattgagtgtatgtaaacttctgacccactgggaatgt | |
| | gatgaaagaaataaaagctgaaatgaatcattctctctactattattctgatatttcacattct | |
| | taaaataaagtggtgatcctaactgacctaagacagggaatttttactaggattaaatgtca | |
| | ggaattgtgaaaaagtgagtttaaatgtatttggctaaggtgtatgtaaacttccgacttcaa | |
| | ctgtatagggatcctctagctagatagttattaatagtaatcaattacggggtcattagttcat | |
| | agcccatatatggagttccgcgttacataacttacggtaaatggcccgcctggctgaccg | |
| | cccaacgacccccgcccattgacgtcaataatgacgtatgttcccatagtaacgccaata | |
| | gggactttccattgacgtcaatgggtggagtatttacggtaaactgcccacttggcagtac | |
| | atcaagtgtatcatatgccaagtacgccccctattgacgtcaatgacggtaaatggcccg | |
| | cctggcattatgcccagtacatgaccttatgggactttcctacttggcagtacatctacgtat | |
| | tagtcatcgctattaccatggtgatgcggttttggcagtacatcaatgggcgtggatagcg | |
| | gtttgactcacggggatttccaagtctccaccccattgacgtcaatgggagtttgttttggc | |
| | accaaaatcaacgggactttccaaaatgtcgtaacaactccgccccattgacgcaaatg | |
| | ggcggtaggcgtgtacggtgggaggtctatataagcagagctggtttagtgaaccgtca | |
| | gatctctggctaactagagaacccactgcgccaccatggtgagcaagggcgaggagct | |
| | gttcaccggggtggtgcccatcctggtcgagctggacggcgacgtaaacggccacaa | |
| | gttcagcgtgtccggcgagggcgagggcgatgccacctacggcaagctgaccctgaa | |
| | gttcatctgcaccaccggcaagctgcccgtgccctggcccaccctcgtgaccaccctga | |
| | cctacggcgtgcagtgcttcagccgctaccccgaccacatgaagcagcacgacttcttc | |
| | aagtccgccatgcccgaaggctacgtccaggagcgcaccatcttcttcaaggacgacg | |
| | gcaactacaagacccgcgccgaggtgaagttcgagggcgacaccctggtgaaccgca | |
| | tcgagctgaagggcatcgacttcaaggaggacggcaacatcctggggcacaagctgg | |
| | agtacaactacaacagccacaacgtctatatcatggccgacaagcagaagaacggcat | |
| | caaggtgaacttcaagatccgccacaacatcgaggacggcagcgtgcagctcgccga | |
| | ccactaccagcagaacacccccatcggcgacggccccgtgctgctgcccgacaacca | |
| | ctacctgagcacccagtccgccctgagcaaagaccccaacgagaagcgcgatcacat | |
| | ggtcctgctggagttcgtgaccgccgccgggatcactctcggcatggacgagctgtaca | |
| | agtaacaactttattatacatagttgatggccggccgcttcgagcagacatgataagatac | |
| | attgatgagtttggacaaaccacaactagaatgcagtgaaaaaaatgctttatttgtgaaat | |
| | ttgtgatgctattgctttatttgtaaccattataagctgcaataaacaagttaacaacaacaat | |
| | tgcattcattttatgtttcaggttcagggggaggtgtgggaggttttttaaagcaagtaaaa | |
| | cctctacaaatgtggtagtcgacctcgagggggggcccggtacccagcttttgttcccttt | |
| | agtgagggttaattgcgcgcttggcgtaatcatggtcatagctgtttcctgtgtgaaattgtt | |
| | atccgctcacaattccacacaacatacgagccggaagcataaagtgtaaagcctggggt | |
| | gcctaatgagtgagctaactcacattaattgcgttgcgctcactgcccgctttccagtcgg | |
| | gaaacctgtcgtgccagctgcattaatgaatcggccaacgcgcggggagaggcggttt | |
| | gcgtattgggcgctcttccgcttcctcgctcactgactcgctgcgctcggtcgttcggctg | |
| | cggcgagcggtatcagctcactcaaaggcggtaatacggttatccacagaatcagggg | |
| | ataacgcaggaaagaacatgtgagcaaaaggccagcaaaaggccaggaaccgtaaa | |
| | aaggccgcgttgctggcgtttttccataggctccgcccccctgacgagcatcacaaaaat | |
| | cgacgctcaagtcagaggtggcgaaacccgacaggactataaagataccaggcgtttc | |
| | cccctggaagctccctcgtgcgctctcctgttccgaccctgccgcttaccggatacctgtc | |
| | cgcctttctcccttcgggaaccgtggcgctttctcatagctcacgctgtaggtatctcagtt | |
| | cggtgtaggtcgttcgctccaagctgggctgtgtgcacgaaccccccgttcagcccgac | |
| | cgctgcgccttatccggtaactatcgtcttgagtccaacccggtaagacacgacttatcgc | |
| | cactggcagcagccactggtaacaggattagcagagcgaggtatgtaggcggtgctac | |
| | agagttcttgaagtggtggcctaactacggctacactagaagaacagtatttggtatctgc | |
| | gctctgctgaagccagttaccttcggaaaaagagttggtagctcttgatccggcaaacaa | |
| | accaccgctggtagcggtggtttttttgtttgcaagcagcagattacgcgcagaaaaaaa | |
| | ggatctcaagaagatcctttgatcttttctacggggtctgacgctcagtggaacgaaaact | |
| | cacgttaagggattttggtcatgagattatcaaaaaggatcttcacctagatccttttaaatt | |
| | aaaaatgaagttttaaatcaatctaaagtatatatgagtaaacttggtctgacagttaccaat | |
| | gcttaatcagtgaggcacctatctcagcgatctgtctatttcgttcatccatagttgcctgac | |
| | tccccgtcgtgtagataactacgatacgggagggcttaccatctggccccagtgctgca | |
| | atgataccgcgagacccacgctcaccggctccagatttatcagcaataaaccagccagc | |
| | cggaagggccgagcgcagaagtggtcctgcaactttatccgcctccatccagtctattaa | |
| | ttgttgccgggaagctagagtaagtagttcgccagttaatagtttgcgcaacgttgttgcc | |
| | attgctacaggcatcgtggtgtcacgctcgtcgtttggtatggcttcattcagctccggttc | |
| | ccaacgatcaaggcgagttacatgatcccccatgttgtgcaaaaaagcggttagctcctt | |
| | cggtcctccgatcgttgtcagaagtaacttggccgcagtgttatcactcatggttatggca | |
| | gcactgcataattctcttactgtcatgccatccgtaagatgcttttctgtgactggtgagtact | |
| | caaccaagtcattctgagaatagtgtatgcggcgaccgagttgctcttgcccggcgtcaa | |
| | tacgggataataccgcgccacatagcagaactttaaaagtgctcatcattggaaaacgtt | |
| | cttcggggcgaaaactctcaaggatcttaccgctgttgagatccagttcgatgtaaccca | |
| | ctcgtgcacccaactgatcttcagcatcttttactttcaccagcgtttctgggtgagcaaaa | |
| | acaggaaggcaaaatgccgcaaaaaagggaataagggcgacacggaaatgttgaata | |
| | ctcatactcttcctttttcaatattattgaagcatttatcagggttattgtctcatgagcggat | |
| | acatatttgaatgtatttagaaaaataaacaaataggggttccgcgcacatttccccgaaaa | |
| | gtgccacctgacgcgccctgtagcggcgcattaagcgcggcgggagtggtggttacg | |
| | cgcagcgtgaccgctacacttgccagcgccctagcgcccgctcctttcgctttcttccctt | |
| | cctttctcgccacgttcgccggctttccccgtcaagctctaaatcgggggctccctttagg | |
| | gttccgatttagtgctttacggcacctcgaccccaaaaaacttgattagggtgatggttcac | |
| | gtagtgggccatcgccctgatagacggtttttcgccctttgacgttggagtccacgttcttt | |
| | aatagtggactcttgttccaaactggaacaacactcaaccctatctcggtctattcttttgatt | |
| | tataagggattttgccgatttcggcctattggttaaaaaatgagctgatttaacaaaaattta | |
| | acgcgaattttaacaaaatattaacgcttacaatttccattcgccattcaggctgcgcaact | |
| | gttgggaagggcgatcggtgcgggcctcttcgctattacgccagctggcgaaaggggg | |
| | atgtgctgcaaggcgattaagttgggtaacgccagggttttcccagtcacgacgttgtaa | |
| | aacgacggccagtgagcgcgcgtaatacgactcactatagggcgaattggagctcgga | |
| | tccc | |

Exemplary DNA that can be used in the donor plasmid can be found in Table 3 listed below:

TABLE 3
Exemplary donor DNA sequences

| Plasmid | Sequence | SEQ ID NO. |
|---|---|---|
| pTERT | tcagtccaggatggtccggaagtctgtagtcagggctgggtcggcggcggcctcg | SEQ ID NO. 10 |
| (bolded = | | |
| reading | agggcggccagtgtggccccagggagccgctggcacaatcgctctctggctgccc | |
| frame 3′- | | |
| >5′) | ggagcggccccagaagacagctgtaggtaacgcggtgacgaaccagcttgagca | |
| | ggaaggcgtggaggcagagccactctgcggcttcagagggaaacgggccggag | |
| | gcgcccctggctcccagggacatccctgcgtttctggctttcaggagcgagtagca | |
| | gcaggacgcggtgtcggagacaagccggaggaaaaatgaggggttcctcgcaa | |
| | gcggctgacggaagggcagctgcagcacacacgcgtggaacctgtaggcctgca | |
| | gcaggaagatcttgtaaacgttggcgagcaccgtcggaagactgttcacctgcag | |
| | gtccagaaggatcccgtggcactttagccgcaagaccgccaagagcttgcggcgc | |
| | atgttcctcccgggcttgaagccctggttgaaggtgagactcgctctgatcgaggtc | |
| | cgggcgtaactggcatagtcgcagtgcacctccagcgtgcgggtgtccagcagca | |
| | gcccgcaccaggggaacaggcagtgtgccggcagctgcagtggggccgggccgc | |
| | cccgggcgccgtcctccacagggaagttcacggccgtcttccgcaagttggccagg | |
| | cagccgtactcgggcacgccgcggaccagggtcctgagaaaggctttggctcgtg | |
| | tcaggtgaggggtcaccagcaggaagtcgtccaccaagcgcaggagcaccccgt | |
| | cacgctggatcccggggaagagtctgttttccatgtccccgtagcacaggctgcag | |
| | agcagcgtggacagaatggagccctgtgggatcccctgacactggacgtaggacc | |
| | tgcccccgatccggatgacgtggtttcgcagcaggctcaggaaaagctccaggag | |
| | acggctgccggcctcgttcagagagcagctctgctcgatgaccacggcgtccctca | |
| | gcgggccggttgcctgcaggtgtgccacaaactgtctcatgtaaggctggaggtct | |
| | gcgaaggtggacacgtgggtttggaaggacttgcgcacgtgcccgcgggcggtcc | |
| | tccggagcacggcgcactggcgcacgcagtacgtgctctcgtggggccggatcac | |
| | gttggcgaccacctccagcagcctgtcctgagggagggcgtcgtaggcccccgtc | |
| | acgtccaccttgacaaagtacagcgggggggccgggtcctgggcccgcaggggc | |
| | agcacaaaggcccgccaggccctgtggatgtcacccacgcccagcacggaggcc | |
| | cccaggaggcgcgggcgccgcgcccgctcgtagttcagcacagcaaacagcgtct | |
| | tgacctgccccgtgagacgccggaccttcttgtctccggggcctgtcctggctcgca | |
| | cgacgttcgccatgttcacgatgggccgcagcccgtcgggtttggggacgaagcg | |
| | gagcttggaggtcagtacagcgggcctggcctcccggcgtcgcctgatctctgcttc | |
| | cgacagttctcgaagccgcacacgatctaagtgttgcctgatgcctgcgctctgcag | |
| | ccggctccagatgcgcttccggaagaagaagagccggttcttctgaaacgtggtct | |
| | ccgtgacataaaaaaacgacctgagcagctcgaccacgtacgtgcccatcaacca | |
| | gcgcaggaacttggccagaatggcctctctcagacggtgctcggcggcctggaca | |
| | tggcgagcgtccgggctccggcgcagccagatgcagtcttgcactttcatcttccac | |
| | atcagctcctgcagcgagagcctggcgtgcttccccagggagacgagcttcttcac | |
| | attcttcagaaagcgccgcttgttgtgcggggagccccagaggcccgggggcacc | |
| | agccggtgaagacaggcccgcaggaggcggtacacctgccaggggctgctgtgc | |
| | tggcggagcagctggactaggcgcctcgaggccgagtccccctcggcggccgctg | |
| | ccagccgctctggagcgagggggcccactttgtggtcgcacacctgaccgccgga | |
| | ccccgagagggtcgccgcagcccgcaacgggcagtgcgccctgagaagcgcctt | |
| | gtaggggcaccgcgcgtggttcttaagcagctcccggaacaggggcctcatccgc | |
| | cagtagcgtgcgggcacgcggcgcgcccctggctgcccgggctttgaggccagaa | |
| | agatgacctccacgagcctcccggcccccgccaggctgggcggcagggagcaga | |
| | gcaggaacgagcggcgcagcccttccttgctccccgagcagtagaggaagcgctt | |
| | ggtctcggcagcgcacacatgaggactgggcttgccctggaggtgtggctgccac | |
| | caggatggggggcaggtaccccgcgcaccgcctgcttgaggggaaaggtgccgg | |
| | gttccgggcgcctcaccctcccgagacttggctttcgcagcggctctggtaggtgtc | |
| | atcacgggggggtcgctgtcgctcggcccaggcgccctgccctggtgggcccggg | |
| | acgcctgcccttcgggaccctgttcaggctccgggacgtgcctgggcctcttggctg | |
| | gaagtggatgtccccccgctctgccccgacgtcgcctcacacettgcgccctctgct | |
| | cctggggtcgcccggcttccccattgcaaacggggcgcgtgagtccgaggccagt | |
| | cggggtctggcccttgtgtcgcatgggccgagcctccgctgcggtgtagaggtcat | |
| | agagtggcggcccgcacacctggtaggcgcaactegggggcaccagcaggtaca | |
| | gcgcgcagcgcgccaacaggtgggtgagcacgtcgtcgcccacgcggcgcagca | |
| | gcagcccccacgcgccgctcccgcgcagtgtgtcggtcacggtgttgggcaggta | |
| | gctgcgcacgctggtcgtgaaggccacgggcggcccgccgcgagccccgtccag | |
| | cagcgcgaagccaaaggccagcacgttcctegcgccgcgctcgcagagcctctgc | |
| | acgaccctggccaccagctccttcaggcaggacacctggcggaaggacggggag | |
| | gcaggaggcggctgcgcgtcccagggcacgcacaccaggcactgcgccaccagc | |
| | gcgcggtaggccgccgggtccccgcgccgaacaagccgccggccctcagggccc | |
| | aggcgccgcacgaaggtggccagcggcagcacctgcctgtagcggtcccggagc | |
| | agggagcgcacggcccggcaccggggcgcgcgcggcatcgccgagaggaggga | |
| | g | |
| GAPDH | TGGAATCTAATCCTAATGAAACTGGCGAGTATTTATT | SEQ ID NO. 11 |
| homologous | | |
| arm #1 (3′- | GAACGCTTTGGTGTCACTGTTTCCAGCAACTTTTCGG | |
| >5′; | | |
| underlined | TGGTGGCTCTGGTCCCTGTTGTGTGGTGTGCACTGGT | |
| indicates a | | |
| silent | GATCATTGCACAGACACACAGGGTTACAAGCAACTT | |
| mutation | | |
| from C -> G) | GCCCAAAGTAATGGCACCTCAAGTAGCAGAACCAGG | |
| | GCCTCCATCCAGGTTGTCAGTGCTGTCTCTGCTTCTTC | |
| | CCACTAAGCCTGTGCGCATTCGCAGGTGACAGAAGA | |
| | CATGGAAATAAAACCACCCCAACCTTTCCCTTGGGCT | |
| | AACCTACTGTAACTTCTCTGAACAATCTCTGAGCTGG | |
| | CACTGGCACAGCTGTACCACAGATGGCAATCTCTTGG | |
| | GCACTGAGGCTCACAGCTGTGACAGGGCAGAGCTGG | |
| | AATCCACTCCTGGCAGTCAGGCCGTAGCCAAATGGA | |
| | CTTCCTGTGCCAGTGCCCCCCTCCAGGGAGCTGCAGA | |
| | CAGAATGGTGTCTTGGGTGCAAGCAGCCAGGCCATTC | |
| | CTGGGTTCAGCCACGGCAGTCTGGAGCTGTAGCACCC | |
| | AGAGGCCTCACTAAGGCAACACGAGGACAGGTGGCT | |
| | CTTCAGAGGAGCCCAGGGTGGAGTTTCCTCAGCAAG | |
| | CTCTAGTTCTCCTTGGCCCCAGCCCGGCTTCCCTCCTC | |
| | TTTATCCCAGGCTCTAGAGTGAGAACGACAGGAGGC | |
| | TGGGTACAGTGTACTTTATTGATGGTACATGACGAGG | |
| | CAGGTCTCCCTAAGCCCCTCCCCTTCTTCCGGGGGTC | |
| | TGGGATGGAAACTGGAAGTCAGGAGATGCTCGGTGT | |
| | GTTGGGGGATCGAGTTGGGGCTGTGACTCCCCAGCA | |
| | GCTGAGGGCCTCTCTCCTCCTCGCGTGCTCTTGCTGG | |
| | 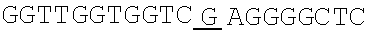 | |
| GAPDH | CTCCTTGGAGGCCATGTGGACCATGAGGTCCACCACC | SEQ ID NO. 12 |
| homologous | | |
| arm #2 (3′- | CTGTTGCTGTAGCCAAATTCATTGTCGTACCTGAAAG | |
| >5′) | | |
| | GAGAGCAAGAGTTAAGTGTGGTTTTCCCGAGCAGCC | |
| | ACCAGGGGGCACCAGACTTGATCACATTCTGAAGCA | |
| | TCATCCCTCCCCACCCGCCACATCCCCCAGCACCCTA | |
| | CCAGGAAATGAGCTTGACGAAGTGGTCGTTGAGGGC | |
| | AATGCCAGCCCCAGCATCAAAAGTGGAAGAGTGAGT | |
| | GTCACTGTTGAAGTCACAGGACACAACCTGGGGAAG | |
| | GAAGAGGGCCAATTACGGAACCCAGTCTTGGTCAGT | |
| | GGGGTCGTGTGGACACCAAGTTCCCTGACCTATCAGT | |
| | ACCTGGTCCTCAGTGTAGCCCAGGATGCCCTTGAGGG | |
| | GGCCCTCGGACGCCTGCTTCACCACCTTCTTGATGTC | |
| | ATCATATTTTGCCTGATGAGGTAAAAACCGATGGTTA | |
| | TTCAAGTACCAATCCTACAACCCCTCAGCCCCCCAAA | |
| | GCTGTTCCAACCCAAACCTACAGGTTTCTCCAGGCGG | |
| | CAGGTCAGATCCACAACCGACACGTTGGGGGTGGGG | |
| | ACACGGAAGGCCATGCCAGTGAGCTTCCTGGGGACA | |
| | CGAGGAAGCAAGCAGAGTCAGCACCACCCCTGCTGG | |
| | TGCCCCAGACACCCAGGGCAGGAGAGCAGCAAAGAG | |
| | CCTCACCCGTTGAGCTCAGGGATGACCTTGCCCACAG | |
| | CCTTGGCAGCGCCGGTAGAAGCAGGGATGATGTTCT | |
| | GGGCAGCCCCTCGGCCATCACGCCACAGCTTCCCAGA | |
| | CGGGCCATCCACAGTCTTCTGGGTGGCAGTGATGGCA | |
| | TGGACCGTGGTCTGGAGACAAGGAGGGGGAGTGAAG | |
| | GCCCTGCCTAGCGCTTGTGACGCC | |
| P2A (3′-> 5′) | GGGGCCGGGGTTCTCCTCCACGTCGCCGGCCTGCTTC | SEQ ID NO. 13 |
| | AGCAGGCTGAAGTTGGTGGCTCCGCTTCC | |
| Rosa26 (5′- | Gggcagcatcctcctgctgagagccgggggagggccaggcccacgtcccgagagc | SEQ ID NO. 14 |
| >3′) | | |
| | aagcgcgaggagacggaggaggtgacccttccctcccccggggcccggtggtgagg | |
| | ggaggtctctcttttctgtcgcacccttaccttgtcccaggcctgggcccgggctgcggc | |
| | gcacggcactcccggtaggcagcaggactcgagttaggcccagcgcggcgccacgg | |
| | cgtttcctggccgggaatggcccgtgcccgtgaggtgggggtggggggcaaaaaggc | |
| | ggagcgagccaaaggcggtgaggggggagggccagggaaggaggggggggccg | |
| | gcactactgtgttggcggactggcgggactggggctgcgtgagtctctgagcgcaggc | |
| | gggcggcggccgcccctcccccggcggcggcggcggcggcggcggcggcggca | |
| | gcagctcactcagcccgctgcccgagcggaaacgccactgaccgcacggggattccc | |
| | agcgccggcgccaggggcacccgggacacgccccctcccgccgcgccattggccc | |
| | ctccgcccaccgtctcgcacccattggccagctccccgccaatcageggaagccgccg | |
| | gggccgcctagagaagaggctgtgctctggggctccggctcctcagagagcctcggct | |
| | ag | |
| cMyc | atgcccctcaacgtcagcttcaccaacaggaactatgacctcgactacgactcggtgca | SEQ ID NO. 15 |
| reading | | |
| frame (5′- | gccttatttctactgcgacgaggaggagaacttctaccagcagcagcagcagagcgag | |
| >3′) | | |
| | ctgcagccgccggcacccagcgaggatatctggaagaaattcgagttgctgcccaccc | |
| | cgcccctgtccccgagccgccgctccgggctctgctcgccctcgtacgtcgcggtcgc | |
| | gtccttctcccccaggggagacgatgacggcggcggcggcagcttttccacggccga | |
| | ccagttggagatggtgaccgagctgctgggaggagacatggtgaaccagagcttcatc | |
| | tgcgaccccgacgacgagaccttcatcaaaaacattatcatccaggactgtatgtggag | |
| | cggcttctcagccgccgccaagctcgtgtcagagaagctggcctcctaccaggctgcg | |
| | cgcaaagacagcggcagcccgattcccgcccgcgggcacggcggctactccacctc | |
| | cagcttgtacctgcaggacctgagcgccgccgcctccgaatgcatcgacccctcggtg | |
| | gtcttcccctacccgctcaacgacagcagctcgcccaagccctgcgcctcccccgactc | |
| | caccgccttctccccgtccteggactctctgctctcctccgccgagtcctccccacgggc | |
| | cagtcccgaacccttggctctccacgaggagacaccacccaccaccagcagcgactct | |
| | gaggaagaacaagaggatgaggaagaaattgatgttgtttctgtggaaaagaggcagc | |
| | cccctgccaaaaggtcggaatcggggtcaccctctgctggaggccacagcaaacctcc | |
| | tcacagccccctggtccttaagagatgccatgtgtccacgcaccagcacaattatgcagc | |
| | gcccccctccaccaggaaggactatccctctgccaagagggctaagttggacagtggc | |
| | agggtcctgaaacagatcagcaacaaccgaaaatgtgccagccccaggtcctcggac | |
| | acggaggagaatgacaagaggcggacacacaatgtcttggaacgccagaggagaaa | |
| | cgagctgaaacgcagcttttttgcccttcgagaccagatcccagagttggagaacaatga | |
| | aaaagcccccaaggtagttatccttaaaaaagccacagcttacatcctgtctgtccaagc | |
| | agaggagcaaaagctcgtgtcagaaaaagacgtgctgcggaagaggcgagaacagtt | |
| | gaaactcaaacttgaacagctacggaactcttgcccataa | |
| IR/DR | cagttgaagtcggaagtttacatacacttaagttggagtcattaaaactcgtttttcaactac | SEQ ID NO. 16 |
| sequence #1 | | |
| (inverted | tccacaaatttcttgttaacaaacaatagttttggcaagtcagttaggacatctactttgtgca | |
| repeats/direct | | |
| repeats; 5′- | tgacacaagtcatttttccaacaattgtttacagacagattatttcacttataattcactgtatc | |
| >3′) | | |
| | acaattccagtgggtcagaagtttacatacactaa | |
| IR/DR | ttgagtgtatgtaaacttctgacccactgggaatgtgatgaaagaaataaaagctgaaatg | SEQ ID NO. 17 |
| sequence #2 | | |
| (inverted | aatcattctctctactattattctgatatttcacattcttaaaataaagtggtgatcctaactgac | |
| repeats/direct | | |
| repeats; 5′- | ctaagacagggaatttttactaggattaaatgtcaggaattgtgaaaaagtgagtttaaatg | |
| >3′) | | |
| | tatttggctaaggtgtatgtaaacttccgacttcaactg | |
| IR/DR | cagttgaagtcggaagtttacatacacttaagttggagtcattaaaactcgtttttcaactac | SEQ ID NO. 18 |
| sequence #3 | | |
| (inverted | tccacaaatttcttgttaacaaacaatagttttggcaagtcagttaggacatctactttgtgca | |
| repeats/direct | | |
| repeats; 5′- | tgacacaagtcatttttccaacaattgtttacagacagattatttcacttataattcactgtatc | |
| >3′) | | |
| | acaattccagtgggtcagaagtttacatacactaa | |
| IR/DR | ttgagtgtatgtaaacttctgacccactgggaatgtgatgaaagaaataaaagctgaaatg | SEQ ID NO. 19 |
| sequence #4 | | |
| (inverted | aatcattctctctactattattctgatatttcacattcttaaaataaagtggtgatcctaactgac | |
| repeats/direct | | |
| repeats; 5′- | ctaagacagggaatttttactaggattaaatgtcaggaattgtgaaaaagtgagtttaaatg | |
| >3′) | | |
| | tatttggctaaggtgtatgtaaacttccgacttcaactg | |
| pEF1alpha | ctggagtaccacgtggtgtgtagaacgaacttagataaaagaactgaatggaaaaagcc | SEQ ID NO. 20 |
| promoter | | |
| | aagaggtcagcgcaggagcctgttcgccaggctgttggtcagcaaactgagccactcg | |
| | cggattctcggccctgaagaccgccccgaggcccctgccgccgeggttccccggcctt | |
| | ttacgcctccctctccagccggaaggggcgcggacggcaagggcggtggagaagcc | |
| | ccggcagaggaagcggctccaggggttagtccttaacctgcgagaccgcaagactga | |
| | ggaccggtggggtactgccgggtaagggctcccatctcggtccaactttgcagttaagc | |
| | tacgaccggttcagggaagattctggacgcctccccgccgccccaccccccagctgtg | |
| | cggagagtaattcatacaaaaggaggactctcctcagccagggaaatcccagggaccg | |
| | tcgataaactcccactaaacctagaaccgagtgagcgctcgaccccgcctcccaccca | |
| | ccagcagtcgtcatcctcctggttgagaggagcatgcgccgggcgccgtgtgctcgtca | |
| | gtgggctgaacgcacatcgcccacggtccccgaagatggggggaggggacggcggt | |
| | ggaaccggtgccgggtggaggtggcgcggggtaaactgggaaagtggtgtcgtgtgc | |
| | tggctccgcccttttccccgaggggggggaggaccatatataagcgccgtggtccccg | |
| | cgaacgttctttttcgcaacgggtttgccgccaggacacaggtgagtacgggtgtggcct | |
| | ccgtccgcatggcctccgccggtggccacggccttagcgtgcctcccggccccccgc | |
| | gcgtagagggctctgcgccctggtcctgattccgagctgcgggcggggggaggtgga | |
| | gaactcgaggccctccgctctcgcggttccctaccgcgtgcccggtggcggcctgctg | |
| | gggcgccgtggccgccgcgtgcgatccgcgccttcgcgcccgccccgtctgtcttcgc | |
| | gctcgagttcggccggcagccgcgtgttactgccagggcggcccagccgaggccgc | |
| | ccaaggtggcctccgtgcccggcggtcccggggcagacggccgcggcggggcggg | |
| | gcgggcggtcggagacccagcgccccgcccgggcgggctgcgagcgcggaaagat | |
| | ggccgctcccggtctcaagatggaggacgcggacggcgggcggcgcacaaagggc | |
| | cgggcggtgctgcccgcgccgcttcccgggactcgcggcccgggtgggggagggg | |
| | ctgggcgccggggcggccccgttcgctgggtgtaggccgggcagggcccgcaggg | |
| | cgccggtgctaatgctccctggagtttgcccttggctgttggggttgtgcctgagtctccg | |
| | gtcgtcgggacagtagtttaaagaaggtttttgtcgtcttaggtgtcgtgaaagccatcgct | |
| | aaaagctaagcaaa | |
| BGH poly A | ctgtgccttctagttgccagccatctgttgtttgcccctcccccgtgccttccttgaccctgg | SEQ ID NO. 21 |
| sequence | | |
| | aaggtgccactcccactgtcctttcctaataaaatgaggaaattgcatcgcattgtctgagt | |
| | aggtgtcattctattctggggggtggggggggcaggacagcaagggggaggattgg | |
| | gaagacaatagcaggcatgctggggatgcggtgggctctatgg | |
| CMV promoter | tagttattaatagtaatcaattacggggtcattagttcatagcccatatatggagttccgcgtt | SEQ ID NO. 22 |
| | acataacttacggtaaatggcccgcctggctgaccgcccaacgacccccgcccattga | |
| | cgtcaataatgacgtatgttcccatagtaacgccaatagggactttccattgacgtcaatg | |
| | ggtggagtatttacggtaaactgcccacttggcagtacatcaagtgtatcatatgccaagt | |
| | acgccccctattgacgtcaatgacggtaaatggcccgcctggcattatgcccagtacatg | |
| | accttatgggactttcctacttggcagtacatctacgtattagtcatcgctattaccatggtg | |
| | atgcggttttggcagtacatcaatgggcgtggatagcggtttgactcacggggatttcca | |
| | agtctccaccccattgacgtcaatgggagtttgttttggcaccaaaatcaacgggactttc | |
| | caaaatgtcgtaacaactccgccccattgacgcaaatgggcggtaggcgtgtacggtg | |
| | ggaggtctatataagcagagctggtttagtgaaccgtcagatc | |
| EGFP coding | atggtgagcaagggcgaggagctgttcaccggggtggtgcccatcctggtcgagctgg | SEQ ID NO. 23 |
| sequence | | |
| | acggcgacgtaaacggccacaagttcagcgtgtccggcgagggcgagggcgatgcc | |
| | acctacggcaagctgaccctgaagttcatctgcaccaccggcaagctgcccgtgccctg | |
| | gcccaccctcgtgaccaccctgacctacggcgtgcagtgcttcagccgctaccccgac | |
| | cacatgaagcagcacgacttcttc | |
| SV40 poly A | cagacatgataagatacattgatgagtttggacaaaccacaactagaatgcagtgaaaaa | SEQ ID NO. 24 |
| sequence | | |
| | aatgctttatttgtgaaatttgtgatgctattgctttatttgtaaccattataagctgcaataaac | |
| | aagttaacaacaacaattgcattcattttatgtttcaggttcagggggaggtgtgggaggtt | |
| | ttttaaagcaagtaaaacctctacaaatgtggta | |
| pUC origin | tttccataggctccgcccccctgacgagcatcacaaaaatcgacgctcaagtcagaggt | SEQ ID NO. 25 |
| of replication | | |
| | ggcgaaacccgacaggactataaagataccaggcgtttccccctggaagctccctcgt | |
| | gcgctctcctgttccgaccctgccgcttaccggatacctgtccgcctttctcccttcggga | |
| | accgtggcgctttctcatagctcacgctgtaggtatctcagttcggtgtaggtcgttcgctc | |
| | caagctgggctgtgtgcacgaaccccccgttcagcccgaccgctgcgccttatccggta | |
| | actatcgtcttgagtccaacccggtaagacacgacttatcgccactggcagcagccact | |
| | ggtaacaggattagcagagcgaggtatgtaggcggtgctacagagttcttgaagtggtg | |
| | gcctaactacggctacactagaagaacagtatttggtatctgcgctctgctgaagccagtt | |
| | accttcggaaaaagagttggtagctcttgatccggcaaacaaaccaccgctggtagcgg | |
| | tggtttttttgtttgcaagcagcagattacgcgcagaaaaaaaggatctcaa | |
| Ampicillin | ttaccaatgcttaatcagtgaggcacctatctcagcgatctgtctatttcgttcatccatagtt | SEQ ID NO. 26 |
| coding | | |
| sequence | gcctgactccccgtcgtgtagataactacgatacgggagggcttaccatctggccccag | |
| | tgctgcaatgataccgcgagacccacgctcaccggctccagatttatcagcaataaacc | |
| | agccagccggaagggccgagcgcagaagtggtcctgcaactttatccgcctccatcca | |
| | gtctattaattgttgccgggaagctagagtaagtagttcgccagttaatagtttgcgcaacg | |
| | ttgttgccattgctacaggcatcgtggtgtcacgctcgtcgtttggtatggcttcattcagct | |
| | ccggttcccaacgatcaaggcgagttacatgatcccccatgttgtgcaaaaaagcggtta | |
| | gctccttcggtcctccgatcgttgtcagaagtaacttggccgcagtgttatcactcatggtt | |
| | atggcagcactgcataattctcttactgtcatgccatccgtaagatgcttttctgtgactggt | |
| | gagtactcaaccaagtcattctgagaatagtgtatgcggcgaccgagttgctcttgcccg | |
| | gcgtcaatacgggataataccgcgccacatagcagaactttaaaagtgctcatcattgga | |
| | aaacgttcttcggggcgaaaactctcaaggatcttaccgctgttgagatccagttcgatgt | |
| | aacccactcgtgcacccaactgatcttcagcatcttttactttcaccagcgtttctgggtga | |
| | gcaaaaacaggaaggcaaaatgccgcaaaaaagggaataagggcgacacggaaatg | |
| | ttgaatactcat | |
| tata box | tata | SEQ ID NO. 27 |
| Kozak | gccacc | SEQ ID NO. 28 |

### Example 5: In Silico sgRNA Design

Disclosed herein are methods to design sgRNA with optimal targeting efficiency in silico.

To design with a minimal off-target effect (creating a knock-out in other loci), sgRNAs with a high on-target score (>70) were designed using Synthego (https://design.synthego.com/#/), as shown in FIG. 4C.

What is claimed is:

1. A method for amplifying animal primary cells, comprising:
introducing into the genome of the animal primary cells a first genetic modification, and a second genetic modification of a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes;
selecting a master cell line from the cells having the first genetic modification and the second genetic modification.

2. A method for amplifying animal primary cells, comprising:
introducing into the genome of the animal primary cells a first genetic modification, and a second genetic modification of a gene or homolog thereof selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A;
selecting a master cell line from the cells having the first genetic modification and the second genetic modification.

3. The method of claim 1 or 2, further comprising growing a subset of cells from the master cell line to a desired cell density, collecting the subset of cells after reaching the desired cell density and forming the collected cells into meat.

4. The method of claim 1 or 2, wherein the first genetic modification increases the expression of a functional telomere reverse transcriptase (“TERT”) protein.

5. The method of claim 1 or 2, wherein the first genetic modification comprises a gene knock-in of a genetic construct expressing a functional TERT protein.

6. The method of claim 5, wherein the gene knock-in comprises a transfection with the genetic construct.

7. The method of claim 5, wherein the gene knock-in is created by using a clustered regularly-interspaced short palindromic repeats-Cas9 (“CRISPR/Cas9”) and a guide RNA targeting the gene or a sleeping beauty transposon.

8. The method according to any one of claims 4-6, wherein the TERT protein is constitutively overexpressed.

9. The method of claim 1 or 2, wherein the first genetic modification is located at a genomic safe harbor site.

10. The method of claim 9, wherein the genomic safe harbor site is selected from the group consisting of Rosa26, pHI1, GAPDH, Pifs501, ACTB, AAVS1, HPRT, TBP, HMBS, and CEP112.

11. The method of claim 1 or 2, wherein the animal primary cells are selected from the group consisting of cartilage cells, liver cells, heart cells, kidney cells, bladder cells, and lung cells.

12. The method of claim 1 or 2, wherein the animal primary cells are selected from the group consisting of fibroblast cells, muscle cells, fat cells, and endothelial cells.

13. The method of claim 1 or 2, wherein the animal primary cells are extracted from an animal species selected from the group consisting of pig, cow, chicken, turkey, sheep, goat, and fish.

14. The method of claim 1 or 2, wherein the second genetic modification comprises knocking-out a gene or homolog thereof, wherein the gene is selected from the group consisting of ATRX, PTEN, TP53, RBT, MAP2K4, CDKN1A (p21), CDKN1B (p27), and ARID1A.

15. The method of claim 1, wherein the second genetic modification is a mutation of a gene or homolog thereof, wherein the gene is selected from the group consisting of ATRX, PTEN, TP53, RB1, MAP2K4, CDKN1A (p21), CDKN1B (p27), and ARID1A.

16. The method of claim 14, wherein the mutation is created by using a CRISPR/Cas9 and a guide RNA targeting the gene.

17. The method of claim 15, wherein the guide RNA comprises a single guide RNA (sgRNA) of 17-23 nucleotides.

18. The method of claim 15, wherein the mutation is a frameshift mutation.

19. A method for amplifying animal primary cells to make meat, comprising:
introducing into the genome of the animal primary cells a first genetic modification;
selecting an immortal primary cell line from the animal primary cells having the first genetic modification;
introducing into the genome of the immortal primary cell line a second genetic modification, wherein the second genetic modification comprises a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes;
selecting a master cell line of the immortal primary cell having the first genetic modification and the second genetic modification; and
growing a subset of cells from the master cell line to a desired cell density.

20. A method for amplifying animal primary cells to make meat, comprising:
introducing into the genome of the animal primary cells a first genetic modification;
selecting an immortal primary cell line from the animal primary cells having the first genetic modification;
introducing into the genome of the immortal primary cell line a second genetic modification, wherein the second genetic modification comprises a gene or homolog thereof selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A;
selecting a master cell line of the immortal primary cell having the first genetic modification and the second genetic modification; and
growing a subset of cells from the master cell line to a desired cell density.

21. The method of claim 19 or 20, further comprising collecting the subset of cells after reaching the desired cell density and forming the collected cells into meat.

22. The method of claim 19 or 20, wherein the first genetic modification increases the expression of a functional TERT protein.

23. The method of claim 19 or 20 wherein the first genetic modification comprises a gene knock-in of a genetic construct expressing a functional TERT protein.

24. The method of claim 23, wherein the gene knock-in comprises a transfection with the genetic construct.

25. The method of claim 23, wherein the gene knock-in is created by using a CRISPR/Cas9 and a guide RNA targeting the gene or a sleeping beauty transposon.

26. The method according to any one of claims 23-25, wherein the TERT protein is constitutively overexpressed.

27. The method of claim 19 or 20, wherein the first genetic modification is located at a genomic safe harbor site.

28. The method of claim 27, wherein the genomic safe harbor site is selected from the group consisting of Rosa26, pHI1, GAPDH, Pifs501, ACTB, AAVS1, HPRT, TBP, HMBS, and CEP112.

29. The method of claim 19 or 20, wherein the animal primary cells are selected from the group consisting of cartilage cells, liver cells, heart cells, kidney cells, bladder cells, and lung cells.

30. The method of claim 19 or 20, wherein the animal primary cells are selected from the group consisting of fibroblast cells, muscle cells, fat cells, and, endothelial cells.

31. The method of claim 19 or 20, wherein the animal primary cells are extracted from an animal species selected from the group consisting of pig, cow, chicken, turkey, sheep, goat, and fish.

32. The method of claim 19 or 20, wherein the second genetic modification comprises knocking-out a gene or homolog thereof, wherein the gene is selected from the group consisting of ATRX, PTEN, TP53, RB1, MAP2K4, CDKN1A (p21), CDKN1B (p27), and ARID1A.

33. The method of claim 19 or 20, wherein the second genetic modification is a mutation of a gene or homolog thereof, wherein the gene is selected from the group consisting of ATRX, PTEN, TP53, RB1,MAP2K4, CDKN1A (p21), CDKN1B (p27), and ARID1A.

34. The method of claim 33, wherein the mutation is created by using a CRISPR/Cas9 and a guide RNA targeting the gene.

35. The method of claim 33, wherein the guide RNA comprises a sgRNA of 17-23 nucleotides.

36. The method of claim 33, wherein the mutation is a frameshift mutation.

37. A master cell line derived from animal primary cells, wherein the master cell line comprises a first genetic modification, and a second genetic modification of a gene selected from the group consisting of tumor suppressor genes, phosphatase genes, chromatin remodeler genes, genes associated with DNA replication, genes associated with cell cycle progression, genes involved in AKT/PKB/PI3K/mTOR pathway, kinase family genes and cadherin family genes.

38. A master cell line derived from animal primary cells, wherein the master cell line comprises a first genetic modification, and a second genetic modification of a gene or homolog thereof selected from the group consisting of: FLCN, MED12, NCOR1, CYSLTR2, BRCA1, BRCA2, KMT2D, AURKB, ALOX12B, ATM, ATR, NF1, NOTCH1, KMT2A, BCOR, ROS1, NTRK1, ERBB4, PDCD1, IGF1R, ARID1A, CIC, SHDA, ERCC5, CREBBP, BARD1, BRIP1, CCNE1, CDK12, CHEK1, CHEK2, FANCA, FANCB, FANCC, FANCD2, FANCE, FANCF, FANCG, FANC1, FANCL, FANCM, FBXW7, MCPH1, MDM2, MRE11A, MYCN, NBN, PALB2, PIK3CA, RAD50, RAD51, RAD51B, RAD51C, RAD51D, RAD54L, SDHA, SDHAF2, SDHB, SDHC, SDHD, SLX4, MUC16, DST, TTN, STMN1, CSF1, GRN, LGALS1, ATP7B, GLI1, IGF2BP3, GGT1, FSCN1, SLC29A1, S100A6, PCA3, TYRO3, PLA2G10, RPRD1B, NAV2, TSPAN31, ZC3H7B, RBL1, RB2, E2F, CDH11, CLK1, DYRK2, DYRK1A, DYRK1B, MAP2K1, MAP2K3, MAP2K6, EGFR, DAXX, NALCN, APOB, CELSR1, MYH9, ROCK1, ROCK2, ATRX, PTEN, TP53, RB1, MAP2K4, CDKN1A (p21), CDKN1B (p27), and ARID1A.

39. The master cell line of claim 37 or 38, wherein the first genetic modification increases the expression of a functional TERT protein.

40. The master cell line of claim 37 or 38, wherein the first genetic modification comprises a gene knock-in of a genetic construct expressing a functional TERT protein.

41. The master cell line of claim 40, wherein the gene knock-in comprises a transfection with the genetic construct.

42. The master cell line of claim 40, wherein the gene knock-in is created by using a CRISPR/Cas9 and a guide RNA targeting the gene or a sleeping beauty transposon.

43. The master cell line according to any one of claims 39-41, wherein the TERT protein is constitutively overexpressed.

44. The master cell line of claim 37 or 38, wherein the first genetic modification is located at a genomic safe harbor site.

45. The master cell line of claim 44, wherein the genomic safe harbor site is selected from the group consisting of Rosa26, pHI1, GAPDH, Pifs501, ACTB, AAVS1, HPRT, TBP, HMBS, and CEP112.

46. The master cell line of claim 37 or 38, wherein the animal primary cells are selected from the group consisting of cartilage cells, liver cells, heart cells, kidney cells, bladder cells, and lung cells.

47. The master cell line of claim 37 or 38, wherein the animal primary cells are selected from the group consisting of fibroblast cells, muscle cells, fat cells, and endothelial cells.

48. The master cell line of claim 37 or 38, wherein the animal primary cells are extracted from an animal species selected from the group consisting of pig, cow, chicken, turkey, sheep, goat, and fish.

49. The master cell line of claim 37 or 38, wherein the second genetic modification comprises knocking-out a gene or homolog thereof, wherein the gene is selected from the group consisting of ATRX, PTEN, TP53, RB1, MAP2K4, CDKN1A (p21), CDKN1B (p27), and ARID1A.

50. The master cell line of claim 37 or 38, wherein the second genetic modification is a mutation of a gene or homolog thereof, wherein the gene is selected from the group consisting of ATRX, PTEN, TP53, RBIl, MAP2K4, CDKN1A (p21), CDKN1B (p27), and ARID1A.

51. The master cell line of claim 50, wherein the mutation is created by using a CRISPR/Cas9 and a guide RNA targeting the gene.

52. The master cell line of claim 51, wherein the guide RNA comprises a sgRNA of 17-23 nucleotides.

53. The master cell line of claim 52, wherein the mutation is a frameshift mutation.

54. A method for rendering animal primary cells immortal, comprising: introducing into the animal primary cell a genetic modification rendering the animal primary cell immortal, wherein the genetic modification is located at a genomic safe harbor site.

55. The method of claim 54, wherein the genomic safe harbor site is selected from the group consisting of Rosa26, pHI1, GAPDH, Pifs501, ACTB, AAVS1, HPRT, TBP, HMBS, and CEP112.

56. The method of claim 54, wherein the genetic modification increases the expression of a functional TERT protein.

57. The method of claim 54, wherein the genetic modification comprises a gene knock-in of a genetic construct expressing a functional TERT protein.

58. The method of claim 54, wherein the gene knock-in comprises a transfection with the genetic construct.

59. The method of claim 57, wherein the gene knock-in is created by using a CRISPR/Cas9 and a guide RNA targeting the gene or a sleeping beauty transposon.

60. The method according to any one of claims 54-59, wherein the TERT protein is constitutively overexpressed.

61. The method of claim 54, wherein the animal primary cells are selected from the group consisting of cartilage cells, liver cells, heart cells, kidney cells, bladder cells, and lung cells.

62. The method of claim 54, wherein the animal primary cells are selected from the group consisting of fibroblast cells, muscle cells, fat cells, and endothelial cells.

63. The method of claim 54, wherein the animal primary cells are extracted from an animal species selected from the group consisting of pig, cow, chicken, turkey, sheep, goat and fish.

64. An immortal primary cell line derived from animal primary cells, wherein the immortal primary cell line comprises a genetic modification rendering the animal primary cells immortal; and wherein the genetic modification is located at a genomic safe harbor site.

65. The immortal primary cell line of claim 64, wherein the genomic safe harbor site is selected from the group consisting of Rosa26, pHI1, GAPDH, Pifs501, ACTB, AAVS1, HPRT, TBP, HMBS, and CEP112.

66. The immortal primary cell line of claim 64, wherein the genetic modification increases the expression of a functional TERT protein.

67. The immortal primary cell line of claim 64, wherein the genetic modification comprises a gene knock-in of a genetic construct expressing a functional TERT protein.

68. The immortal primary cell line of claim 67, wherein the gene knock-in comprises a transfection with the genetic construct.

69. The immortal primary cell line of claim 67, wherein the gene knock-in is created by using a CRISPR/Cas9 and a guide RNA targeting the gene or a sleeping beauty transposon.

70. The master cell line according to any one of claims 66-69, wherein the TERT protein is constitutively overexpressed.

71. The immortal primary cell line of claim 64, wherein the animal primary cells are selected from the group consisting of cartilage cells, liver cells, heart cells, kidney cells, bladder cells, and lung cells.

72. The immortal primary cell line of claim 64, wherein the animal primary cells are selected from the group consisting of fibroblast cells, muscle cells, fat cells and endothelial cells.

73. The immortal primary cell line of claim 64, wherein the animal primary cells are extracted from an animal species selected from the group consisting of pig, cow, chicken, turkey, sheep, goat and fish.

74. A method for amplifying animal primary cells, comprising:
introducing into the animal primary cells a genetic modification rendering the animal primary cell highly proliferative, wherein the genetic modification is located at a genomic safe harbor site;
selecting a master cell line of the animal primary cell having the genetic modification.

75. The method of claim 74, further comprising growing a subset of cells from the master cell line to a desired cell density, collecting the subset of cells after reaching the desired cell density and forming the collected cells into meat.

76. The method of claim 74, wherein the genomic safe harbor site is selected from the group consisting of Rosa26, pHI1, GAPDH, Pifs501, ACTB, AAVS1, HPRT, TBP, HMBS, and CEP112.

77. The method of claim 74, wherein the genetic modification increases the expression of a proto-oncogene.

78. The method of claim 77, wherein the proto-oncogene expresses a functional cMyc protein.

79. The method of claim 74, wherein the genetic modification comprises a gene knock-in of a genetic construct expressing a functional cMyc protein.

80. The method of claim 79, wherein the gene knock-in comprises a transfection with the genetic construct.

81. The method according to any one of claims 78-80, wherein the cMyc protein is constitutively overexpressed.

82. The method of claim 79, wherein the gene knock-in is created by using a CRISPR/Cas9 and a guide RNA targeting the gene or a sleeping beauty transposon.

83. The method of claim 74, wherein the animal primary cells are selected from the group consisting of cartilage cells, liver cells, heart cells, kidney cells, bladder cells, and lung cells.

84. The method of claim 74, wherein the animal primary cells are selected from the group consisting of fibroblast cells, muscle cells, fat cells, and endothelial cells.

85. The method of claim 74, wherein the animal primary cells are extracted from an animal species selected from the group consisting of pig, cow, chicken, turkey, sheep, goat, and fish.

86. A master cell line derived from animal primary cells, wherein the master cell line comprises a genetic modification rendering the master cell line highly proliferative; and wherein the genetic modification is located at a genomic safe harbor site.

87. The master cell line of claim 86, wherein the genomic safe harbor site is selected from the group consisting of Rosa26, pHI1, GAPDH, Pifs501, ACTB, AAVS1, HPRT, TBP, HMBS, and CEP112.

88. The master cell line of claim 86, wherein the genetic modification increases the expression of a proto-oncogene.

89. The master cell line of claim 88, wherein the proto-oncogene expresses a functional cMyc protein.

90. The master cell line of claim 86, wherein the genetic modification comprises a gene knock-in of a genetic construct expressing a functional cMyc protein.

91. The master cell line of claim 90, wherein the gene knock-in comprises a transfection with the genetic construct.

92. The master cell line according to any one of claim 89-91, wherein the cMyc protein is constitutively overexpressed.

93. The method of claim 90, wherein the gene knock-in is created by using a CRISPR/Cas9 and a guide RNA targeting the gene or a sleeping beauty transposon.

94. The master cell line of claim 86, wherein the animal primary cells are selected from the group consisting of cartilage cells, liver cells, heart cells, kidney cells, bladder cells, and lung cells.

95. The master cell line of claim 86, wherein the animal primary cells are selected from the group consisting of fibroblast cells, muscle cells, fat cells, and endothelial cells.

96. The master cell line of claim 86, wherein the animal primary cells are extracted from an animal species selected from the group consisting of pig, cow, chicken, turkey, sheep, goat, and fish.


SEQUENCE LISTING (46 sequences)

SEQ ID NO: 1 - skipped

SEQ ID NO: 2 - RNA, 20 nt, synthetic construct
  source 1..20: mol_type = other RNA
ctccaaggagtaagagcccc

SEQ ID NO: 3 - RNA, 20 nt, synthetic construct
  source 1..20: mol_type = other RNA
caacagggtggtggacctca

SEQ ID NO: 4 - RNA, 20 nt, synthetic construct
  source 1..20: mol_type = other RNA
ggtggattatggtcttcgaa

SEQ ID NO: 5 - RNA, 19 nt, synthetic construct
  source 1..19: mol_type = other RNA
tgacataatgtagtaaatc

SEQ ID NO: 6 - RNA, 20 nt, synthetic construct
  source 1..20: mol_type = other RNA
tgaaagtaagttgaatacac

SEQ ID NO: 7 - DNA, 6637 bp, synthetic construct
  source 1..6637: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagctttggaatctaatcctaatgaaactggcgagtatttattgaacgctttggtgtcactgtttccagcaacttttcggtggtggctctggtccctgttgtgtggtgtgcactggtgatcattgcacagacacacagggttacaagcaacttgcccaaagtaatggcacctcaagtagcagaaccagggcctccatccaggttgtcagtgctgtctctgcttcttcccactaagcctgtgcgcattcgcaggtgacagaagacatggaaataaaaccaccccaacctttcccttgggctaacctactgtaacttctctgaacaatctctgagctggcactggcacagctgtaccacagatggcaatctcttgggcactgaggctcacagctgtgacagggcagagctggaatccactcctggcagtcaggccgtagccaaatggacttcctgtgccagtgcccccctccagggagctgcagacagaatggtgtcttgggtgcaagcagccaggccattcctgggttcagccacggcagtctggagctgtagcacccagaggcctcactaaggcaacacgaggacaggtggctcttcagaggagcccagggtggagtttcctcagcaagctctagttctccttggccccagcccggcttccctcctctttatcccaggctctagagtgagaacgacaggaggctgggtacagtgtactttattgatggtacatgacgaggcaggtctccctaagcccctccccttcttccgggggtctgggatggaaactggaagtcaggagatgctcggtgtgttgggggatcgagttggggctgtgactccccagcagctgagggcctctctcctcctcgcgtgctcttgctggggttggtggtcgaggggctctcagtccaggatggtccggaagtctgtagtcagggctgggtcggcggcggcctcgagggcggccagtgtggccccagggagccgctggcacaatcgctctctggctgcccggagcggccccagaagacagctgtaggtaacgcggtgacgaaccagcttgagcaggaaggcgtggaggcagagccactctgcggcttcagagggaaacgggccggaggcgcccctggctcccagggacatccctgcgtttctggctttcaggagcgagtagcagcaggacgcggtgtcggagacaagccggaggaaaaatgaggggttcctcgcaagcggctgacggaagggcagctgcagcacacacgcgtggaacctgtaggcctgcagcaggaagatcttgtaaacgttggcgagcaccgtcggaagactgttcacctgcaggtccagaaggatcccgtggcactttagccgcaagaccgccaagagcttgcggcgcatgttcctcccgggcttgaagccctggttgaaggtgagactcgctctgatcgaggtccgggcgtaactggcatagtcgcagtgcacctccagcgtgcgggtgtccagcagcagcccgcaccaggggaacaggcagtgtgccggcagctgcagtggggccgggccgccccgggcgccgtcctccacagggaagttcacggccgtcttccgcaagttggccaggcagccgtactcgggcacgccgcggaccagggtcctgagaaaggctttggctcgtgtcaggtgaggggtcaccagcaggaagtcgtccaccaagcgcaggagcaccccgtcacgctggatcccggggaagagtctgttttccatgtccccgtagcacaggctgcagagcagcgtggacagaatggagccctgtgggatcccctgacactggacgtaggacctgcccccgatccggatgacgtggtttcgcagcaggctcaggaaaagctccaggagacggctgccggcctcgttcagagagcagctctgctcgatgaccacggcgtccctcagcgggccggttgcctgcaggtgtgccacaaactgtctcatgtaaggctggaggtctgcgaaggtggacacgtgggtttggaaggacttgcgcacgtgcccgcgggcggtcctccggagcacggcgcactggcgcacgcagtacgtgctctcgtggggccggatcacgttggcgaccacctccagcagcctgtcctgagggagggcgtcgtaggcccccgtcacgtccaccttgacaaagtacagcgggggggccgggtcctgggcccgcaggggcagcacaaaggcccgccaggccctgtggatgtcacccacgcccagcacggaggcccccaggaggcgcgggcgccgcgcccgctcgtagttcagcacagcaaacagcgtcttgacctgccccgtgagacgccggaccttcttgtctccggggcctgtcctggctcgcacgacgttcgccatgttcacgatgggccgcagcccgtcgggtttggggacgaagcggagcttggaggtcagtacagcgggcctggcctcccggcgtcgcctgatctctgcttccgacagttctcgaagccgcacacgatctaagtgttgcctgatgcctgcgctctgcagccggctccagatgcgcttccggaagaagaagagccggttcttctgaaacgtggtctccgtgacataaaaaaacgacctgagcagctcgaccacgtacgtgcccatcaaccagcgcaggaacttggccagaatggcctctctcagacggtgctcggcggcctggacatggcgagcgtccgggctccggcgcagccagatgcagtcttgcactttcatcttccacatcagctcctgcagcgagagcctggcgtgcttccccagggagacgagcttcttcacattcttcagaaagcgccgcttgttgtgcggggagccccagaggcccgggggcaccagccggtgaagacaggcccgcaggaggcggtacacctgccaggggctgctgtgctggcggagcagctggactaggcgcctcgaggccgagtccccctcggcggccgctgccagccgctctggagcgagggggcccactttgtggtcgcacacctgaccgccggaccccgagagggtcgccgcagcccgcaacgggcagtgcgccctgagaagcgccttgtaggggcaccgcgcgtggttcttaagcagctcccggaacaggggcctcatccgccagtagcgtgcgggcacgcggcgcgcccctggctgcccgggctttgaggccagaaagatgacctccacgagcctcccggcccccgccaggctgggcggcagggagcagagcaggaacgagcggcgcagcccttccttgctccccgagcagtagaggaagcgcttggtctcggcagcgcacacatgaggactgggcttgccctggaggtgtggctgccaccaggatggggggcaggtaccccgcgcaccgcctgcttgaggggaaaggtgccgggttccgggcgcctcaccctcccgagacttggctttcgcagcggctctggtaggtgtcatcacgggggggtcgctgtcgctcggcccaggcgccctgccctggtgggcccgggacgcctgcccttcgggaccctgttcaggctccgggacgtgcctgggcctcttggctggaagtggatgtccccccgctctgccccgacgtcgcctcacaccttgcgccctctgctcctggggtcgcccggcttccccattgcaaacggggcgcgtgagtccgaggccagtcggggtctggcccttgtgtcgcatgggccgagcctccgctgcggtgtagaggtcatagagtggcggcccgcacacctggtaggcgcaactcgggggcaccagcaggtacagcgcgcagcgcgccaacaggtgggtgagcacgtcgtcgcccacgcggcgcagcagcagcccccacgcgccgctcccgcgcagtgtgtcggtcacggtgttgggcaggtagctgcgcacgctggtcgtgaaggccacgggcggcccgccgcgagccccgtccagcagcgcgaagccaaaggccagcacgttcctcgcgccgcgctcgcagagcctctgcacgaccctggccaccagctccttcaggcaggacacctggcggaaggacggggaggcaggaggcggctgcgcgtcccagggcacgcacaccaggcactgcgccaccagcgcgcggtaggccgccgggtccccgcgccgaacaagccgccggccctcagggcccaggcgccgcacgaaggtggccagcggcagcacctgcctgtagcggtcccggagcagggagcgcacggcccggcaccggggcgcgcgcggcatcgccgagaggagggagggggccggggttctcctccacgtcgccggcctgcttcagcaggctgaagttggtggctccgcttccctccttggaggccatgtggaccatgaggtccaccaccctgttgctgtagccaaattcattgtcgtacctgaaaggagagcaagagttaagtgtggttttcccgagcagccaccagggggcaccagacttgatcacattctgaagcatcatccctccccacccgccacatcccccagcaccctaccaggaaatgagcttgacgaagtggtcgttgagggcaatgccagccccagcatcaaaagtggaagagtgagtgtcactgttgaagtcacaggacacaacctggggaaggaagagggccaattacggaacccagtcttggtcagtggggtcgtgtggacaccaagttccctgacctatcagtacctggtcctcagtgtagcccaggatgcccttgagggggccctcggacgcctgcttcaccaccttcttgatgtcatcatattttgcctgatgaggtaaaaaccgatggttattcaagtaccaatcctacaacccctcagccccccaaagctgttccaacccaaacctacaggtttctccaggcggcaggtcagatccacaaccgacacgttgggggtggggacacggaaggccatgccagtgagcttcctggggacacgaggaagcaagcagagtcagcaccacccctgctggtgccccagacacccagggcaggagagcagcaaagagcctcacccgttgagctcagggatgaccttgcccacagccttggcagcgccggtagaagcagggatgatgttctgggcagcccctcggccatcacgccacagcttcccagacgggccatccacagtcttctgggtggcagtgatggcatggaccgtggtctggagacaaggagggggagtgaaggccctgcctagcgcttgtgacgccggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgct

SEQ ID NO: 8 - DNA, 2863 bp, synthetic construct
  source 1..2863: mol_type = other DNA
tatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctggccatctagagggcagcatcctcctgctgagagccgggggagggccaggcccacgtcccgagagcaagcgcgaggagacggaggaggtgacccttccctcccccggggcccggtggtgaggggaggtctctcttttctgtcgcacccttaccttgtcccaggcctgggcccgggctgcggcgcacggcactcccggtaggcagcaggactcgagttaggcccagcgcggcgccacggcgtttcctggccgggaatggcccgtgcccgtgaggtgggggtggggggcaaaaaggcggagcgagccaaaggcggtgaggggggagggccagggaaggaggggggggccggcactactgtgttggcggactggcgggactggggctgcgtgagtctctgagcgcaggcgggcggcggccgcccctcccccggcggcggcggcggcggcggcggcggcggcagcagctcactcagcccgctgcccgagcggaaacgccactgaccgcacggggattcccagcgccggcgccaggggcacccgggacacgccccctcccgccgcgccattggcccctccgcccaccgtctcgcacccattggccagctccccgccaatcagcggaagccgccggggccgcctagagaagaggctgtgctctggggctccggctcctcagagagcctcggctaggccaccatgcccctcaacgtcagcttcaccaacaggaactatgacctcgactacgactcggtgcagccttatttctactgcgacgaggaggagaacttctaccagcagcagcagcagagcgagctgcagccgccggcacccagcgaggatatctggaagaaattcgagttgctgcccaccccgcccctgtccccgagccgccgctccgggctctgctcgccctcgtacgtcgcggtcgcgtccttctcccccaggggagacgatgacggcggcggcggcagcttttccacggccgaccagttggagatggtgaccgagctgctgggaggagacatggtgaaccagagcttcatctgcgaccccgacgacgagaccttcatcaaaaacattatcatccaggactgtatgtggagcggcttctcagccgccgccaagctcgtgtcagagaagctggcctcctaccaggctgcgcgcaaagacagcggcagcccgattcccgcccgcgggcacggcggctactccacctccagcttgtacctgcaggacctgagcgccgccgcctccgaatgcatcgacccctcggtggtcttcccctacccgctcaacgacagcagctcgcccaagccctgcgcctcccccgactccaccgccttctccccgtcctcggactctctgctctcctccgccgagtcctccccacgggccagtcccgaacccttggctctccacgaggagacaccacccaccaccagcagcgactctgaggaagaacaagaggatgaggaagaaattgatgttgtttctgtggaaaagaggcagccccctgccaaaaggtcggaatcggggtcaccctctgctggaggccacagcaaacctcctcacagccccctggtccttaagagatgccatgtgtccacgcaccagcacaattatgcagcgcccccctccaccaggaaggactatccctctgccaagagggctaagttggacagtggcagggtcctgaaacagatcagcaacaaccgaaaatgtgccagccccaggtcctcggacacggaggagaatgacaagaggcggacacacaatgtcttggaacgccagaggagaaacgagctgaaacgcagcttttttgcccttcgagaccagatcccagagttggagaacaatgaaaaagcccccaaggtagttatccttaaaaaagccacagcttacatcctgtctgtccaagcagaggagcaaaagctcgtgtcagaaaaagacgtgctgcggaagaggcgagaacagttgaaactcaaacttgaacagctacggaactcttgcccataaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 9 - DNA, 10286 bp, synthetic construct
  source 1..10286: mol_type = other DNA
tatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagaagctagatctggccatctagactggagtaccacgtggtgtgtagaacgaacttagataaaagaactgaatggaaaaagccaagaggtcagcgcaggagcctgttcgccaggctgttggtcagcaaactgagccactcgcggattctcggccctgaagaccgccccgaggcccctgccgccgcggttccccggccttttacgcctccctctccagccggaaggggcgcggacggcaagggcggtggagaagccccggcagaggaagcggctccaggggttagtccttaacctgcgagaccgcaagactgaggaccggtggggtactgccgggtaagggctcccatctcggtccaactttgcagttaagctacgaccggttcagggaagattctggacgcctccccgccgccccaccccccagctgtgcggagagtaattcatacaaaaggaggactctcctcagccagggaaatcccagggaccgtcgataaactcccactaaacctagaaccgagtgagcgctcgaccccgcctcccacccaccagcagtcgtcatcctcctggttgagaggagcatgcgccgggcgccgtgtgctcgtcagtgggctgaacgcacatcgcccacggtccccgaagatggggggaggggacggcggtggaaccggtgccgggtggaggtggcgcggggtaaactgggaaagtggtgtcgtgtgctggctccgcccttttccccgagggtgggggaggaccatatataagcgccgtggtccccgcgaacgttctttttcgcaacgggtttgccgccaggacacaggtgagtacgggtgtggcctccgtccgcatggcctccgccggtggccacggccttagcgtgcctcccggccccccgcgcgtagagggctctgcgccctggtcctgattccgagctgcgggcggggggaggtggagaactcgaggccctccgctctcgcggttccctaccgcgtgcccggtggcggcctgctggggcgccgtggccgccgcgtgcgatccgcgccttcgcgcccgccccgtctgtcttcgcgctcgagttcggccggcagccgcgtgttactgccagggcggcccagccgaggccgcccaaggtggcctccgtgcccggcggtcccggggcagacggccgcggcggggcggggcgggcggtcggagacccagcgccccgcccgggcgggctgcgagcgcggaaagatggccgctcccggtctcaagatggaggacgcggacggcgggcggcgcacaaagggccgggcggtgctgcccgcgccgcttcccgggactcgcggcccgggtgggggaggggctgggcgccggggcggccccgttcgctgggtgtaggccgggcagggcccgcagggcgccggtgctaatgctccctggagtttgcccttggctgttggggttgtgcctgagtctccggtcgtcgggacagtagtttaaagaaggtttttgtcgtcttaggtgtcgtgaaagccatcgctaaaagctaagcaaagccaccatgccgcgcgcgccccggtgccgggccgtgcgctccctgctccgggaccgctacaggcaggtgctgccgctggccaccttcgtgcggcgcctgggccctgagggccggcggcttgttcggcgcggggacccggcggcctaccgcgcgctggtggcgcagtgcctggtgtgcgtgccctgggacgcgcagccgcctcctgcctccccgtccttccgccaggtgtcctgcctgaaggagctggtggccagggtcgtgcagaggctctgcgagcgcggcgcgaggaacgtgctggcctttggcttcgcgctgctggacggggctcgcggcgggccgcccgtggccttcacgaccagcgtgcgcagctacctgcccaacaccgtgaccgacacactgcgcgggagcggcgcgtgggggctgctgctgcgccgcgtgggcgacgacgtgctcacccacctgttggcgcgctgcgcgctgtacctgctggtgcccccgagttgcgcctaccaggtgtgcgggccgccactctatgacctctacaccgcagcggaggctcggcccatgcgacacaagggccagaccccgactggcctcggactcacgcgccccgtttgcaatggggaagccgggcgaccccaggagcagagggcgcaaggtgtgaggcgacgtcggggcagagcggggggacatccacttccagccaagaggcccaggcacgtcccggagcctgaacagggtcccgaagggcaggcgtcccgggcccaccagggcagggcgcctgggccgagcgacagcgacccccccgtgatgacacctaccagagccgctgcgaaagccaagtctcgggagggtgaggcgcccggaacccggcacctttcccctcaagcaggcggtgcgcggggtacctgccccccatcctggtggcagccacacctccagggcaagcccagtcctcatgtgtgcgctgccgagaccaagcgcttcctctactgctcggggagcaaggaagggctgcgccgctcgttcctgctctgctccctgccgcccagcctggcgggggccgggaggctcgtggaggtcatctttctggcctcaaagcccgggcagccaggggcgcgccgcgtgcccgcacgctactggcggatgaggcccctgttccgggagctgcttaagaaccacgcgcggtgcccctacaaggcgcttctcagggcgcactgcccgttgcgggctgcggcgaccctctcggggtccggcggtcaggtgtgcgaccacaaagtgggccccctcgctccagagcggctggcagcggccgccgagggggactcggcctcgaggcgcctagtccagctgctccgccagcacagcagcccctggcaggtgtaccgcctcctgcgggcctgtcttcaccggctggtgcccccgggcctctggggctccccgcacaacaagcggcgctttctgaagaatgtgaagaagctcgtctccctggggaagcacgccaggctctcgctgcaggagctgatgtggaagatgaaagtgcaagactgcatctggctgcgccggagcccggacgctcgccatgtccaggccgccgagcaccgtctgagagaggccattctggccaagttcctgcgctggttgatgggcacgtacgtggtcgagctgctcaggtcgtttttttatgtcacggagaccacgtttcagaagaaccggctcttcttcttccggaagcgcatctggagccggctgcagagcgcaggcatcaggcaacacttagatcgtgtgcggcttcgagaactgtcggaagcagagatcaggcgacgccgggaggccaggcccgctgtactgacctccaagctccgcttcgtccccaaacccgacgggctgcggcccatcgtgaacatggcgaacgtcgtgcgagccaggacaggccccggagacaagaaggtccggcgtctcacggggcaggtcaagacgctgtttgctgtgctgaactacgagcgggcgcggcgcccgcgcctcctgggggcctccgtgctgggcgtgggtgacatccacagggcctggcgggcctttgtgctgcccctgcgggcccaggacccggcccccccgctgtactttgtcaaggtggacgtgacgggggcctacgacgccctccctcaggacaggctgctggaggtggtcgccaacgtgatccggccccacgagagcacgtactgcgtgcgccagtgcgccgtgctccggaggaccgcccgcgggcacgtgcgcaagtccttccaaacccacgtgtccaccttcgcagacctccagccttacatgagacagtttgtggcacacctgcaggcaaccggcccgctgagggacgccgtggtcatcgagcagagctgctctctgaacgaggccggcagccgtctcctggagcttttcctgagcctgctgcgaaaccacgtcatccggatcgggggcaggtcctacgtccagtgtcaggggatcccacagggctccattctgtccacgctgctctgcagcctgtgctacggggacatggaaaacagactcttccccgggatccagcgtgacggggtgctcctgcgcttggtggacgacttcctgctggtgacccctcacctgacacgagccaaagcctttctcaggaccctggtccgcggcgtgcccgagtacggctgcctggccaacttgcggaagacggccgtgaacttccctgtggaggacggcgcccggggcggcccggccccactgcagctgccggcacactgcctgttcccctggtgcgggctgctgctggacacccgcacgctggaggtgcactgcgactatgccagttacgcccggacctcgatcagagcgagtctcaccttcaaccagggcttcaagcccgggaggaacatgcgccgcaagctcttggcggtcttgcggctaaagtgccacgggatccttctggacctgcaggtgaacagtcttccgacggtgctcgccaacgtttacaagatcttcctgctgcaggcctacaggttccacgcgtgtgtgctgcagctgcccttccgtcagccgcttgcgaggaacccctcatttttcctccggcttgtctccgacaccgcgtcctgctgctactcgctcctgaaagccagaaacgcagggatgtccctgggagccaggggcgcctccggcccgtttccctctgaagccgcagagtggctctgcctccacgccttcctgctcaagctggttcgtcaccgcgttacctacagctgtcttctggggccgctccgggcagccagagagcgattgtgccagcggctccctggggccacactggccgccctcgaggccgccgccgacccagccctgactacagacttccggaccatcctggactgaacccagctttcttgtacaaagtggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgtggaaggctactcgaaatgtttgacccaagttaaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatctctggctaactagagaacccactgcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaaccgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaacttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggagtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcggatccc

SEQ ID NO: 10 - DNA, 3412 bp, synthetic construct
  source 1..3412: mol_type = other DNA
tcagtccaggatggtccggaagtctgtagtcagggctgggtcggcggcggcctcgagggcggccagtgtggccccagggagccgctggcacaatcgctctctggctgcccggagcggccccagaagacagctgtaggtaacgcggtgacgaaccagcttgagcaggaaggcgtggaggcagagccactctgcggcttcagagggaaacgggccggaggcgcccctggctcccagggacatccctgcgtttctggctttcaggagcgagtagcagcaggacgcggtgtcggagacaagccggaggaaaaatgaggggttcctcgcaagcggctgacggaagggcagctgcagcacacacgcgtggaacctgtaggcctgcagcaggaagatcttgtaaacgttggcgagcaccgtcggaagactgttcacctgcaggtccagaaggatcccgtggcactttagccgcaagaccgccaagagcttgcggcgcatgttcctcccgggcttgaagccctggttgaaggtgagactcgctctgatcgaggtccgggcgtaactggcatagtcgcagtgcacctccagcgtgcgggtgtccagcagcagcccgcaccaggggaacaggcagtgtgccggcagctgcagtggggccgggccgccccgggcgccgtcctccacagggaagttcacggccgtcttccgcaagttggccaggcagccgtactcgggcacgccgcggaccagggtcctgagaaaggctttggctcgtgtcaggtgaggggtcaccagcaggaagtcgtccaccaagcgcaggagcaccccgtcacgctggatcccggggaagagtctgttttccatgtccccgtagcacaggctgcagagcagcgtggacagaatggagccctgtgggatcccctgacactggacgtaggacctgcccccgatccggatgacgtggtttcgcagcaggctcaggaaaagctccaggagacggctgccggcctcgttcagagagcagctctgctcgatgaccacggcgtccctcagcgggccggttgcctgcaggtgtgccacaaactgtctcatgtaaggctggaggtctgcgaaggtggacacgtgggtttggaaggacttgcgcacgtgcccgcgggcggtcctccggagcacggcgcactggcgcacgcagtacgtgctctcgtggggccggatcacgttggcgaccacctccagcagcctgtcctgagggagggcgtcgtaggcccccgtcacgtccaccttgacaaagtacagcgggggggccgggtcctgggcccgcaggggcagcacaaaggcccgccaggccctgtggatgtcacccacgcccagcacggaggcccccaggaggcgcgggcgccgcgcccgctcgtagttcagcacagcaaacagcgtcttgacctgccccgtgagacgccggaccttcttgtctccggggcctgtcctggctcgcacgacgttcgccatgttcacgatgggccgcagcccgtcgggtttggggacgaagcggagcttggaggtcagtacagcgggcctggcctcccggcgtcgcctgatctctgcttccgacagttctcgaagccgcacacgatctaagtgttgcctgatgcctgcgctctgcagccggctccagatgcgcttccggaagaagaagagccggttcttctgaaacgtggtctccgtgacataaaaaaacgacctgagcagctcgaccacgtacgtgcccatcaaccagcgcaggaacttggccagaatggcctctctcagacggtgctcggcggcctggacatggcgagcgtccgggctccggcgcagccagatgcagtcttgcactttcatcttccacatcagctcctgcagcgagagcctggcgtgcttccccagggagacgagcttcttcacattcttcagaaagcgccgcttgttgtgcggggagccccagaggcccgggggcaccagccggtgaagacaggcccgcaggaggcggtacacctgccaggggctgctgtgctggcggagcagctggactaggcgcctcgaggccgagtccccctcggcggccgctgccagccgctctggagcgagggggcccactttgtggtcgcacacctgaccgccggaccccgagagggtcgccgcagcccgcaacgggcagtgcgccctgagaagcgccttgtaggggcaccgcgcgtggttcttaagcagctcccggaacaggggcctcatccgccagtagcgtgcgggcacgcggcgcgcccctggctgcccgggctttgaggccagaaagatgacctccacgagcctcccggcccccgccaggctgggcggcagggagcagagcaggaacgagcggcgcagcccttccttgctccccgagcagtagaggaagcgcttggtctcggcagcgcacacatgaggactgggcttgccctggaggtgtggctgccaccaggatggggggcaggtaccccgcgcaccgcctgcttgaggggaaaggtgccgggttccgggcgcctcaccctcccgagacttggctttcgcagcggctctggtaggtgtcatcacgggggggtcgctgtcgctcggcccaggcgccctgccctggtgggcccgggacgcctgcccttcgggaccctgttcaggctccgggacgtgcctgggcctcttggctggaagtggatgtccccccgctctgccccgacgtcgcctcacaccttgcgccctctgctcctggggtcgcccggcttccccattgcaaacggggcgcgtgagtccgaggccagtcggggtctggcccttgtgtcgcatgggccgagcctccgctgcggtgtagaggtcatagagtggcggcccgcacacctggtaggcgcaactcgggggcaccagcaggtacagcgcgcagcgcgccaacaggtgggtgagcacgtcgtcgcccacgcggcgcagcagcagcccccacgcgccgctcccgcgcagtgtgtcggtcacggtgttgggcaggtagctgcgcacgctggtcgtgaaggccacgggcggcccgccgcgagccccgtccagcagcgcgaagccaaaggccagcacgttcctcgcgccgcgctcgcagagcctctgcacgaccctggccaccagctccttcaggcaggacacctggcggaaggacggggaggcaggaggcggctgcgcgtcccagggcacgcacaccaggcactgcgccaccagcgcgcggtaggccgccgggtccccgcgccgaacaagccgccggccctcagggcccaggcgccgcacgaaggtggccagcggcagcacctgcctgtagcggtcccggagcagggagcgcacggcccggcaccggggcgcgcgcggcatcgccgagaggagggag

SEQ ID NO: 11 - DNA, 900 bp, synthetic construct
  source 1..900: mol_type = other DNA
tggaatctaatcctaatgaaactggcgagtatttattgaacgctttggtgtcactgtttccagcaacttttcggtggtggctctggtccctgttgtgtggtgtgcactggtgatcattgcacagacacacagggttacaagcaacttgcccaaagtaatggcacctcaagtagcagaaccagggcctccatccaggttgtcagtgctgtctctgcttcttcccactaagcctgtgcgcattcgcaggtgacagaagacatggaaataaaaccaccccaacctttcccttgggctaacctactgtaacttctctgaacaatctctgagctggcactggcacagctgtaccacagatggcaatctcttgggcactgaggctcacagctgtgacagggcagagctggaatccactcctggcagtcaggccgtagccaaatggacttcctgtgccagtgcccccctccagggagctgcagacagaatggtgtcttgggtgcaagcagccaggccattcctgggttcagccacggcagtctggagctgtagcacccagaggcctcactaaggcaacacgaggacaggtggctcttcagaggagcccagggtggagtttcctcagcaagctctagttctccttggccccagcccggcttccctcctctttatcccaggctctagagtgagaacgacaggaggctgggtacagtgtactttattgatggtacatgacgaggcaggtctccctaagcccctccccttcttccgggggtctgggatggaaactggaagtcaggagatgctcggtgtgttgggggatcgagttggggctgtgactccccagcagctgagggcctctctcctcctcgcgtgctcttgctggggttggtggtcgaggggctc

SEQ ID NO: 12 - DNA, 900 bp, synthetic construct
  source 1..900: mol_type = other DNA
ctccttggaggccatgtggaccatgaggtccaccaccctgttgctgtagccaaattcattgtcgtacctgaaaggagagcaagagttaagtgtggttttcccgagcagccaccagggggcaccagacttgatcacattctgaagcatcatccctccccacccgccacatcccccagcaccctaccaggaaatgagcttgacgaagtggtcgttgagggcaatgccagccccagcatcaaaagtggaagagtgagtgtcactgttgaagtcacaggacacaacctggggaaggaagagggccaattacggaacccagtcttggtcagtggggtcgtgtggacaccaagttccctgacctatcagtacctggtcctcagtgtagcccaggatgcccttgagggggccctcggacgcctgcttcaccaccttcttgatgtcatcatattttgcctgatgaggtaaaaaccgatggttattcaagtaccaatcctacaacccctcagccccccaaagctgttccaacccaaacctacaggtttctccaggcggcaggtcagatccacaaccgacacgttgggggtggggacacggaaggccatgccagtgagcttcctggggacacgaggaagcaagcagagtcagcaccacccctgctggtgccccagacacccagggcaggagagcagcaaagagcctcacccgttgagctcagggatgaccttgcccacagccttggcagcgccggtagaagcagggatgatgttctgggcagcccctcggccatcacgccacagcttcccagacgggccatccacagtcttctgggtggcagtgatggcatggaccgtggtctggagacaaggagggggagtgaaggccctgcctagcgcttgtgacgcc

SEQ ID NO: 13 - DNA, 66 bp, synthetic construct
  source 1..66: mol_type = other DNA
ggggccggggttctcctccacgtcgccggcctgcttcagcaggctgaagttggtggctccgcttcc

SEQ ID NO: 14 - DNA, 678 bp, synthetic construct
  source 1..678: mol_type = other DNA
gggcagcatcctcctgctgagagccgggggagggccaggcccacgtcccgagagcaagcgcgaggagacggaggaggtgacccttccctcccccggggcccggtggtgaggggaggtctctcttttctgtcgcacccttaccttgtcccaggcctgggcccgggctgcggcgcacggcactcccggtaggcagcaggactcgagttaggcccagcgcggcgccacggcgtttcctggccgggaatggcccgtgcccgtgaggtgggggtggggggcaaaaaggcggagcgagccaaaggcggtgaggggggagggccagggaaggaggggggggccggcactactgtgttggcggactggcgggactggggctgcgtgagtctctgagcgcaggcgggcggcggccgcccctcccccggcggcggcggcggcggcggcggcggcggcagcagctcactcagcccgctgcccgagcggaaacgccactgaccgcacggggattcccagcgccggcgccaggggcacccgggacacgccccctcccgccgcgccattggcccctccgcccaccgtctcgcacccattggccagctccccgccaatcagcggaagccgccggggccgcctagagaagaggctgtgctctggggctccggctcctcagagagcctcggctag

SEQ ID NO: 15 - DNA, 1320 bp, synthetic construct
  source 1..1320: mol_type = other DNA
atgcccctcaacgtcagcttcaccaacaggaactatgacctcgactacgactcggtgcagccttatttctactgcgacgaggaggagaacttctaccagcagcagcagcagagcgagctgcagccgccggcacccagcgaggatatctggaagaaattcgagttgctgcccaccccgcccctgtccccgagccgccgctccgggctctgctcgccctcgtacgtcgcggtcgcgtccttctcccccaggggagacgatgacggcggcggcggcagcttttccacggccgaccagttggagatggtgaccgagctgctgggaggagacatggtgaaccagagcttcatctgcgaccccgacgacgagaccttcatcaaaaacattatcatccaggactgtatgtggagcggcttctcagccgccgccaagctcgtgtcagagaagctggcctcctaccaggctgcgcgcaaagacagcggcagcccgattcccgcccgcgggcacggcggctactccacctccagcttgtacctgcaggacctgagcgccgccgcctccgaatgcatcgacccctcggtggtcttcccctacccgctcaacgacagcagctcgcccaagccctgcgcctcccccgactccaccgccttctccccgtcctcggactctctgctctcctccgccgagtcctccccacgggccagtcccgaacccttggctctccacgaggagacaccacccaccaccagcagcgactctgaggaagaacaagaggatgaggaagaaattgatgttgtttctgtggaaaagaggcagccccctgccaaaaggtcggaatcggggtcaccctctgctggaggccacagcaaacctcctcacagccccctggtccttaagagatgccatgtgtccacgcaccagcacaattatgcagcgcccccctccaccaggaaggactatccctctgccaagagggctaagttggacagtggcagggtcctgaaacagatcagcaacaaccgaaaatgtgccagccccaggtcctcggacacggaggagaatgacaagaggcggacacacaatgtcttggaacgccagaggagaaacgagctgaaacgcagcttttttgcccttcgagaccagatcccagagttggagaacaatgaaaaagcccccaaggtagttatccttaaaaaagccacagcttacatcctgtctgtccaagcagaggagcaaaagctcgtgtcagaaaaagacgtgctgcggaagaggcgagaacagttgaaactcaaacttgaacagctacggaactcttgcccataa

SEQ ID NO: 16 - DNA, 227 bp, synthetic construct
  source 1..227: mol_type = other DNA
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 17 - DNA, 228 bp, synthetic construct
  source 1..228: mol_type = other DNA
ttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 18 - DNA, 227 bp, synthetic construct; identical to SEQ ID NO: 16
  source 1..227: mol_type = other DNA

SEQ ID NO: 19 - DNA, 228 bp, synthetic construct; identical to SEQ ID NO: 17
  source 1..228: mol_type = other DNA

SEQ ID NO: 20 - DNA, 1510 bp, synthetic construct
  source 1..1510: mol_type = other DNA
ctggagtaccacgtggtgtgtagaacgaacttagataaaagaactgaatggaaaaagccaagaggtcagcgcaggagcctgttcgccaggctgttggtcagcaaactgagccactcgcggattctcggccctgaagaccgccccgaggcccctgccgccgcggttccccggccttttacgcctccctctccagccggaaggggcgcggacggcaagggcggtggagaagccccggcagaggaagcggctccaggggttagtccttaacctgcgagaccgcaagactgaggaccggtggggtactgccgggtaagggctcccatctcggtccaactttgcagttaagctacgaccggttcagggaagattctggacgcctccccgccgccccaccccccagctgtgcggagagtaattcatacaaaaggaggactctcctcagccagggaaatcccagggaccgtcgataaactcccactaaacctagaaccgagtgagcgctcgaccccgcctcccacccaccagcagtcgtcatcctcctggttgagaggagcatgcgccgggcgccgtgtgctcgtcagtgggctgaacgcacatcgcccacggtccccgaagatggggggaggggacggcggtggaaccggtgccgggtggaggtggcgcggggtaaactgggaaagtggtgtcgtgtgctggctccgcccttttccccgagggtgggggaggaccatatataagcgccgtggtccccgcgaacgttctttttcgcaacgggtttgccgccaggacacaggtgagtacgggtgtggcctccgtccgcatggcctccgccggtggccacggccttagcgtgcctcccggccccccgcgcgtagagggctctgcgccctggtcctgattccgagctgcgggcggggggaggtggagaactcgaggccctccgctctcgcggttccctaccgcgtgcccggtggcggcctgctggggcgccgtggccgccgcgtgcgatccgcgccttcgcgcccgccccgtctgtcttcgcgctcgagttcggccggcagccgcgtgttactgccagggcggcccagccgaggccgcccaaggtggcctccgtgcccggcggtcccggggcagacggccgcggcggggcggggcgggcggtcggagacccagcgccccgcccgggcgggctgcgagcgcggaaagatggccgctcccggtctcaagatggaggacgcggacggcgggcggcgcacaaagggccgggcggtgctgcccgcgccgcttcccgggactcgcggcccgggtgggggaggggctgggcgccggggcggccccgttcgctgggtgtaggccgggcagggcccgcagggcgccggtgctaatgctccctggagtttgcccttggctgttggggttgtgcctgagtctccggtcgtcgggacagtagtttaaagaaggtttttgtcgtcttaggtgtcgtgaaagccatcgctaaaagctaagcaaa

SEQ ID NO: 21 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 589 bp, synthetic construct
  source 1..589: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatc

SEQ ID NO: 23 - DNA, 255 bp, synthetic construct
  source 1..255: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttc

SEQ ID NO: 24 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 25 - DNA, 589 bp, synthetic construct
  source 1..589: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaaccgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 26 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaacttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 27-28 - skipped (2 entries)

SEQ ID NO: 29 - DNA, 20 bp, synthetic construct
  source 1..20: mol_type = other DNA
ggtggattatggtcttcgaa

SEQ ID NO: 30 - DNA, 95 bp, synthetic construct
  source 1..95: mol_type = other DNA
tttttccacagttgcacagtatcctttcgaagaccataatccaccacagctagaacttatcaaacctttttgtgaagatcttgaccaatggctaa

SEQ ID NO: 31 - DNA, 95 bp, synthetic construct
  source 1..95: mol_type = other DNA
tttttccacagttgcacagtatcctttcgaaaaaccaaatcccccccagctaaaaattattcaacccttttgggaaaaacctggcccatgggtaa

SEQ ID NO: 32 - DNA, 90 bp, synthetic construct
  source 1..90: mol_type = other DNA
ttaactcttgctctcctttcaggtacgacaatgaatttggctacagcaacagggtggtggacctcatggtccacatggcctccaaggagt

SEQ ID NO: 33 - DNA, 100 bp, synthetic construct
  source 1..100: mol_type = other DNA
  misc_difference 39: note = a, c, g, t, unknown or other
ggttaattagggttattttaaccattttaatttaaccanggaaagtaagtggaatcactggtgcagaagcttcatgatttcctggcacactcatcagaag

SEQ ID NO: 34 - protein, 30 aa, synthetic construct
LTLALLSGTTMNLATATGWWTSWSTWPPRS

SEQ ID NO: 35 - protein, 9 aa, synthetic construct
LLLSFQVRQ

SEQ ID NO: 36 - protein, 19 aa, synthetic construct
IWLQQQGGGPHGPHGLQGV

SEQ ID NO: 37 - protein, 30 aa, synthetic construct
LNSCSPFRYDNEFGYSNRVVDLMVHMASKE

SEQ ID NO: 38 - DNA, 90 bp, synthetic construct
  source 1..90: mol_type = other DNA
aagagcccctggaccaccaaccccagcaagagcacgcgaggaggagagaggccctcagctgctggggagtcacagccccaactcgatccc

SEQ ID NO: 39 - protein, 26 aa, synthetic construct
HFYFNSESKLNTLVQKLHDFLAHSSE

SEQ ID NO: 40 - protein, 29 aa, synthetic construct
VAQYPFEDHNPPQLELIKPFCEDLDQWLS

SEQ ID NO: 41 - protein, 36 aa, synthetic construct
LRLSFSTVAQYPFEDHNPPQLELIKPFCEDLDQWLS

SEQ ID NO: 42 - DNA, 106 bp, synthetic construct
  source 1..106: mol_type = other DNA
tgaggttatctttttccacagttgcacagtatcctttcgaagaccataatccaccacagctagaacttatcaaacctttttgtgaagatcttgaccaatggctaag

SEQ ID NO: 43 - DNA, 106 bp, synthetic construct
  source 1..106: mol_type = other DNA
  misc_difference 1: note = a, c, g, t, unknown or other
  misc_difference 7: note = a, c, g, t, unknown or other
  misc_difference 8: note = a, c, g, t, unknown or other
  misc_difference 9: note = a, c, g, t, unknown or other
  misc_difference 10: note = a, c, g, t, unknown or other
  misc_difference 15: note = a, c, g, t, unknown or other
  misc_difference 17: note = a, c, g, t, unknown or other
  misc_difference 18: note = a, c, g, t, unknown or other
  misc_difference 20: note = a, c, g, t, unknown or other
  misc_difference 21: note = a, c, g, t, unknown or other
  misc_difference 24: note = a, c, g, t, unknown or other
  misc_difference 25: note = a, c, g, t, unknown or other
  misc_difference 26: note = a, c, g, t, unknown or other
  misc_difference 28: note = a, c, g, t, unknown or other
  misc_difference 29: note = a, c, g, t, unknown or other
  misc_difference 30: note = a, c, g, t, unknown or other
  misc_difference 31: note = a, c, g, t, unknown or other
  misc_difference 32: note = a, c, g, t, unknown or other
  misc_difference 34: note = a, c, g, t, unknown or other
  misc_difference 37: note = a, c, g, t, unknown or other
  misc_difference 39: note = a, c, g, t, unknown or other
  misc_difference 41: note = a, c, g, t, unknown or other
  misc_difference 46: note = a, c, g, t, unknown or other
  misc_difference 47: note = a, c, g, t, unknown or other
  misc_difference 50: note = a, c, g, t, unknown or other
  misc_difference 57: note = a, c, g, t, unknown or other
  misc_difference 58: note = a, c, g, t, unknown or other
  misc_difference 59: note = a, c, g, t, unknown or other
  misc_difference 60: note = a, c, g, t, unknown or other
  misc_difference 62: note = a, c, g, t, unknown or other
  misc_difference 63: note = a, c, g, t, unknown or other
  misc_difference 66: note = a, c, g, t, unknown or other
  misc_difference 69: note = a, c, g, t, unknown or other
  misc_difference 70: note = a, c, g, t, unknown or other
  misc_difference 71: note = a, c, g, t, unknown or other
  misc_difference 72: note = a, c, g, t, unknown or other
  misc_difference 82: note = a, c, g, t, unknown or other
  misc_difference 88: note = a, c, g, t, unknown or other
  misc_difference 89: note = a, c, g, t, unknown or other
  misc_difference 90: note = a, c, g, t, unknown or other
  misc_difference 91: note = a, c, g, t, unknown or other
  misc_difference 92: note = a, c, g, t, unknown or other
  misc_difference 98: note = a, c, g, t, unknown or other
  misc_difference 101: note = a, c, g, t, unknown or other
  misc_difference 102: note = a, c, g, t, unknown or other
  misc_difference 106: note = a, c, g, t, unknown or other
naaggtnnnnttttncnncnntgnnncnnnnncnttncnanaaccnnaanccccccnnnntnnaanttnnnnaacctttttngaaaannnnngaccantgnntaan

SEQ ID NO: 44 - DNA, 101 bp, synthetic construct
  source 1..101: mol_type = other DNA
  misc_difference 1: note = a, c, g, t, unknown or other
  misc_difference 2: note = a, c, g, t, unknown or other
  misc_difference 4: note = a, c, g, t, unknown or other
  misc_difference 8: note = a, c, g, t, unknown or other
  misc_difference 10: note = a, c, g, t, unknown or other
  misc_difference 11: note = a, c, g, t, unknown or other
  misc_difference 19: note = a, c, g, t, unknown or other
  misc_difference 21: note = a, c, g, t, unknown or other
  misc_difference 22: note = a, c, g, t, unknown or other
  misc_difference 28: note = a, c, g, t, unknown or other
  misc_difference 29: note = a, c, g, t, unknown or other
  misc_difference 30: note = a, c, g, t, unknown or other
  misc_difference 31: note = a, c, g, t, unknown or other
  misc_difference 32: note = a, c, g, t, unknown or other
  misc_difference 33: note = a, c, g, t, unknown or other
  misc_difference 35: note = a, c, g, t, unknown or other
  misc_difference 40: note = a, c, g, t, unknown or other
  misc_difference 42: note = a, c, g, t, unknown or other
  misc_difference 48: note = a, c, g, t, unknown or other
  misc_difference 49: note = a, c, g, t, unknown or other
  misc_difference 53: note = a, c, g, t, unknown or other
  misc_difference 54: note = a, c, g, t, unknown or other
  misc_difference 56: note = a, c, g, t, unknown or other
  misc_difference 58: note = a, c, g, t, unknown or other
  misc_difference 59: note = a, c, g, t, unknown or other
  misc_difference 60: note = a, c, g, t, unknown or other
  misc_difference 61: note = a, c, g, t, unknown or other
  misc_difference 64: note = a, c, g, t, unknown or other
  misc_difference 65: note = a, c, g, t, unknown or other
  misc_difference 66: note = a, c, g, t, unknown or other
  misc_difference 68: note = a, c, g, t, unknown or other
  misc_difference 69: note = a, c, g, t, unknown or other
  misc_difference 70: note = a, c, g, t, unknown or other
  misc_difference 71: note = a, c, g, t, unknown or other
  misc_difference 73: note = a, c, g, t, unknown or other
  misc_difference 75: note = a, c, g, t, unknown or other
  misc_difference 78: note = a, c, g, t, unknown or other
  misc_difference 80: note = a, c, g, t, unknown or other
  misc_difference 82: note = a, c, g, t, unknown or other
  misc_difference 85: note = a, c, g, t, unknown or other
  misc_difference 86: note = a, c, g, t, unknown or other
  misc_difference 87: note = a, c, g, t, unknown or other
  misc_difference 88: note = a, c, g, t, unknown or other
  misc_difference 89: note = a, c, g, t, unknown or other
  misc_difference 91: note = a, c, g, t, unknown or other
  misc_difference 92: note = a, c, g, t, unknown or other
  misc_difference 94: note = a, c, g, t, unknown or other
  misc_difference 96: note = a, c, g, t, unknown or other
  misc_difference 97: note = a, c, g, t, unknown or other
  misc_difference 98: note = a, c, g, t, unknown or other
  misc_difference 99: note = a, c, g, t, unknown or other
  misc_difference 100: note = a, c, g, t, unknown or other
nnanggtntnnttttcccncnntggccnnnnnncnttccnanaccccnncacnncnannnnttnnnannnntntntgngnanatnnnnncnngngnnnnng

SEQ ID NO: 45 - DNA, 107 bp, synthetic construct
  source 1..107: mol_type = other DNA
  misc_difference 1: note = a, c, g, t, unknown or other
  misc_difference 2: note = a, c, g, t, unknown or other
  misc_difference 3: note = a, c, g, t, unknown or other
  misc_difference 11: note = a, c, g, t, unknown or other
  misc_difference 21: note = a, c, g, t, unknown or other
  misc_difference 22: note = a, c, g, t, unknown or other
  misc_difference 26: note = a, c, g, t, unknown or other
  misc_difference 30: note = a, c, g, t, unknown or other
  misc_difference 33: note = a, c, g, t, unknown or other
  misc_difference 38: note = a, c, g, t, unknown or other
  misc_difference 39: note = a, c, g, t, unknown or other
  misc_difference 43: note = a, c, g, t, unknown or other
  misc_difference 48: note = a, c, g, t, unknown or other
  misc_difference 55: note = a, c, g, t, unknown or other
  misc_difference 61: note = a, c, g, t, unknown or other
  misc_difference 62: note = a, c, g, t, unknown or other
  misc_difference 63: note = a, c, g, t, unknown or other
  misc_difference 67: note = a, c, g, t, unknown or other
  misc_difference 68: note = a, c, g, t, unknown or other
  misc_difference 70: note = a, c, g, t, unknown or other
  misc_difference 71: note = a, c, g, t, unknown or other
  misc_difference 72: note = a, c, g, t, unknown or other
  misc_difference 84: note = a, c, g, t, unknown or other
  misc_difference 85: note = a, c, g, t, unknown or other
  misc_difference 88: note = a, c, g, t, unknown or other
  misc_difference 94: note = a, c, g, t, unknown or other
  misc_difference 95: note = a, c, g, t, unknown or other
  misc_difference 96: note = a, c, g, t, unknown or other
  misc_difference 105: note = a, c, g, t, unknown or other
  misc_difference 107: note = a, c, g, t, unknown or other
nnnaggtatcnttttcccccnntggnccanaanccttnngaanaaccntatcccnccccannnaaanntnnncaacccttttgnnaanaacctnnnccatgggtnan

SEQ ID NO: 46 - DNA, 101 bp, synthetic construct
  source 1..101: mol_type = other DNA
ggttaactaaggatattcttaacatttttattttaacagtgaaagtaagttgaatacactggtgcagaagcttcatgatttcctggcacactcatcagaag